(19) United States
(12) Patent Application Publication
(10) Pub. No.: US 2021/0202843 A1
(43) Pub. Date: Jul. 1, 2021

(54) NOVEL ORGANIC ELECTROLUMINESCENT COMPOUND AND ORGANIC ELECTROLUMINESCENT DEVICE

(71) Applicant: NANJING TOPTO SEMICONDUCTOR MATERIAL CO., LTD., Nanjing (CN)

(72) Inventors: Chao QIAN, NANJING (CN); Jun XU, NANJING (CN)

(21) Appl. No.: 16/887,450

(22) Filed: May 29, 2020

Related U.S. Application Data
(63) Continuation of application No. PCT/CN2020/071105, filed on Jan. 9, 2020.

(30) Foreign Application Priority Data
Dec. 31, 2019 (CN) 201911407305.3

(51) Int. Cl. H01L 51/00; C07C 211/61; C09K 11/06; C07D 213/38

(52) U.S. Cl. CPC H01L 51/006; C07C 211/61; C09K 11/06; C07D 213/38; H01L 51/0061; H01L 51/5012; C09K 2211/1007; H01L 51/0058; C09K 2211/1018; H01L 51/0067; C09K 2211/1011

(57) ABSTRACT

The present disclosure discloses a novel organic electroluminescent compound and an organic electroluminescent device, the novel organic electroluminescent compound having a structural formula as follows:

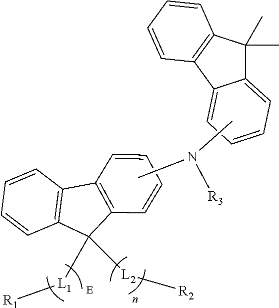

where L₁ and L₂ are phenylene, and L₁ and L₂ can be connected with each other through a single bond or are not connected; R₁ and R₂ are each independently hydrogen, deuterium, a cyano group, a substituted or unsubstituted C₁-C₂₀ straight or branched alkyl group, a substituted or unsubstituted C₃-C₂₀ cycloalkyl group, a substituted or unsubstituted C₃-C₂₀ heterocycloalkyl group, a substituted or unsubstituted C₆-C₃₀ aromatic hydrocarbon group, or a substituted or unsubstituted C₅-C₃₀ heteroaromatic hydrocarbon group; R₃ is a substituted or unsubstituted C₆-C₃₀ aromatic hydrocarbon group, or a substituted or unsubstituted C₅-C₃₀ heteroaromatic hydrocarbon group; and m and n are each independently 0 or 1.

### CROSS-REFERENCE TO RELATED APPLICATIONS

The present application is a Continuation of PCT/CN2020/071105, filed on Jan. 9, 2020, entitled “NOVEL ORGANIC ELECTROLUMINESCENT COMPOUND AND ORGANIC ELECTROLUMINESCENT DEVICE”, which claims convention priority to Chinese Patent Application CN201911407305.3, filed on Dec. 31, 2019, the entirety of which is incorporated herein by reference.

### TECHNICAL FIELD

The present disclosure relates to the technical field of organic electroluminescence, particularly to a novel organic electroluminescent compound and an organic electroluminescent device.

### BACKGROUND ART

Organic electroluminescent devices (Organic Light-emitting Devices, OLED) are spontaneous luminescent devices that utilize the following principle: when an electric field is applied, a fluorescent substance emits light through recombination of holes injected from a positive electrode and electrons injected from a negative electrode. Such self-luminous devices have the characteristics such as low voltage, high brightness, wide visual angle, quick response and good temperature adaptability, and have the advantages of being ultrathin, being able to be manufactured on a flexible panel and so on, thus they are widely applied to fields such as mobile phones, tablet computers, televisions and illumination.

The sandwich-like structure of the organic electroluminescent devices includes electrode material film layers, and an organic functional material sandwiched between different electrode film layers, with different functional materials being superposed together with each other according to purposes, to form the organic electroluminescent devices. When a voltage is applied to electrodes at two ends of an organic electroluminescent device as a current device, and positive and negative charges are generated in the organic layer functional material film layer under the action of an electric field, the positive and negative charges are further compounded in the light-emitting layer to generate light, which process is electroluminescence.

Researches on the improvement of the performance of the organic electroluminescent devices include: reducing a driving voltage of the devices, improving the light-emitting efficiency of the devices, prolonging the service life of the devices and so on. In order to realize the continuous improvement of the performance of the organic electroluminescent devices, not only the structure and the manufacturing process of the organic electroluminescent devices need to be innovated, but also the continuous research and innovation of the organic electroluminescent functional material are required, so as to create organic electroluminescent functional materials with higher performance.

In terms of actual demand of the current organic electroluminescent industry, at present, the development of the organic electroluminescent material is far from enough, and lags behind the requirements of panel manufacturing enterprises.

### SUMMARY

An object of the present disclosure lies in providing a novel organic electroluminescent compound and an organic electroluminescent device for solving the above technical problems.

In order to achieve the above object of the present disclosure, a following technical solution is adopted in the present disclosure:

An novel organic electroluminescent compound, having a structural formula as follows:

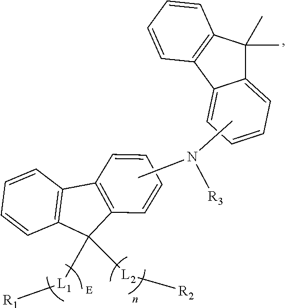

where L₁ and L₂ are phenylene, and L₁ and L₂ can be connected with each other through a single bond or are not connected;

R₁ and R₂ are each independently hydrogen, deuterium, a cyano group, a substituted or unsubstituted C₁-C₂₀ straight or branched alkyl group, a substituted or unsubstituted C₃-C₂₀ cycloalkyl group, a substituted or unsubstituted C₃-C₂₀ heterocycloalkyl group, a substituted or unsubstituted C₆-C₃₀ aromatic hydrocarbon group, or a substituted or unsubstituted C₅-C₃₀ heteroaromatic hydrocarbon group;

R₃ is a substituted or unsubstituted C₆-C₃₀ aromatic hydrocarbon group, or a substituted or unsubstituted C₅-C₃₀ heteroaromatic hydrocarbon group; and

m and n are each independently 0 or 1.

Further, R₁ and R₂ are each independently hydrogen, deuterium, a substituted or unsubstituted C₁-C₂₀ straight or branched alkyl group, or a phenyl group.

Further, R₁ and R₂ are each independently hydrogen, deuterium, a methyl group, an ethyl group, an isopropyl group, a tert-butyl group or a phenyl group, and the methyl group, ethyl group, isopropyl group, tert-butyl group or phenyl group is unsubstituted or a group obtained by substituting at least one hydrogen therein by deuterium.

Further, R₃ is a substituted or unsubstituted phenyl group or a substituted or unsubstituted biphenyl group, and at least one C of the phenyl group and biphenyl group is replaced by N or unreplaced.

Further, R₃ is a phenyl group or a biphenyl group substituted by a C₁-C₂₀ straight or branched alkyl group, or by a C₃-C₂₀ cycloalkyl group, or by a C₃-C₂₀ cycloalkenyl group;

at least one C in the phenyl group and biphenyl group is replaced by N or unreplaced; and

at least one hydrogen in the C₁-C₂₀ straight or branched alkyl group, the C₃-C₂₀ cycloalkyl group and the C₃-C₂₀ cycloalkenyl group is substituted by deuterium or unsubstituted.

Further, R₃ is an unsubstituted phenyl group, or an unsubstituted biphenyl group, or a phenyl group or a biphenyl group substituted by a methyl group, an ethyl group, a propyl group, an isopropyl group, an n-butyl group, a sec-butyl group, an isobutyl group, a tert-butyl group, a neopentyl group, a cyclopropyl group, a cyclobutyl group, a cyclopentyl group, a cyclohexyl group, a cyclopropenyl group, a cyclobutenyl group, a cyclobutadienyl group, a cyclopentenyl group, a cyclopentadienyl group, a cyclohexenyl group, a cyclohexadienyl group or an adamantyl group;

at least one C of the phenyl group and the biphenyl group is replaced by N or unreplaced; and

at least one hydrogen of the methyl group, ethyl group, propyl group, isopropyl group, n-butyl group, sec-butyl group, isobutyl group, tert-butyl group, neopentyl group, cyclopropyl group, cyclobutyl group, cyclopentyl group, cyclohexyl group, cyclopropenyl group, cyclobutenyl group, cyclobutadienyl group, cyclopentenyl group, cyclopentadienyl group, cyclohexenyl group, cyclohexadienyl group and adamantyl group is substituted by deuterium or unsubstituted.

Further, the structural formula of the novel organic electroluminescent compound is represented as follows:

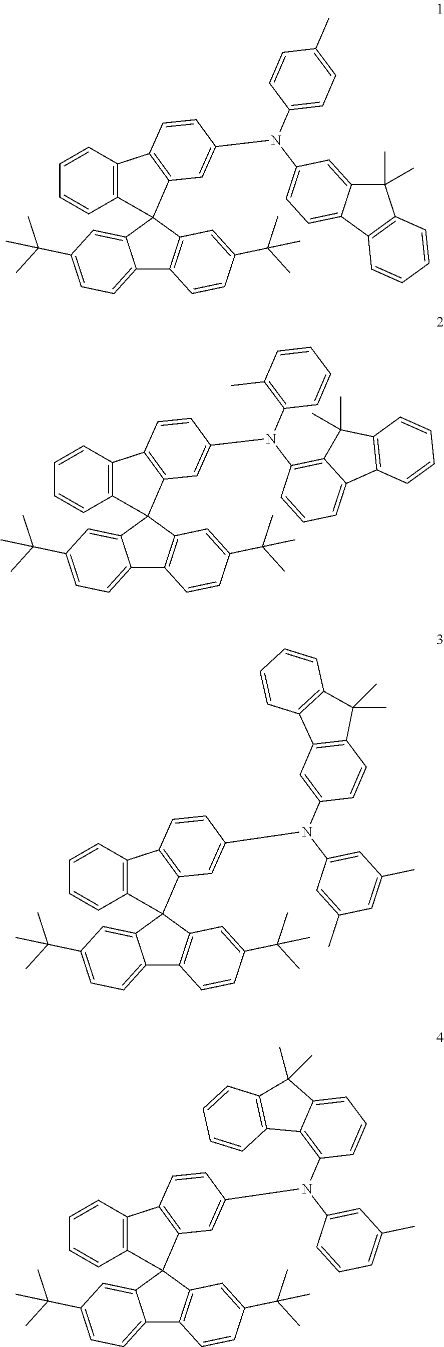

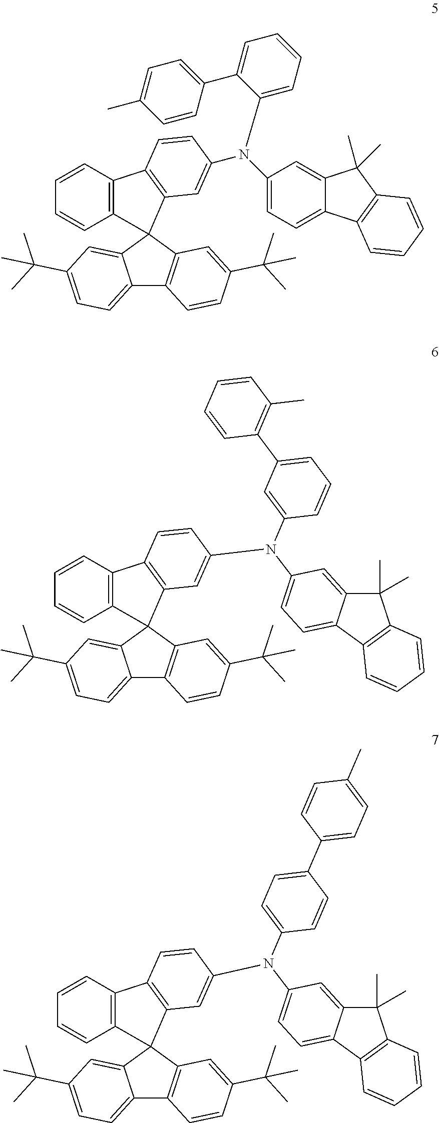 [SMILES: Cc1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1 ; Cc1ccccc1-c1cccc(N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)c1 ; Cc1ccc(-c2ccc(N(c3ccc4c(c3)C(C)(C)c3ccccc3-4)c3ccc4c(c3)C3(c5ccccc5-4)c4cc(C(C)(C)C)ccc4-c4ccc(C(C)(C)C)cc43)cc2)cc1]

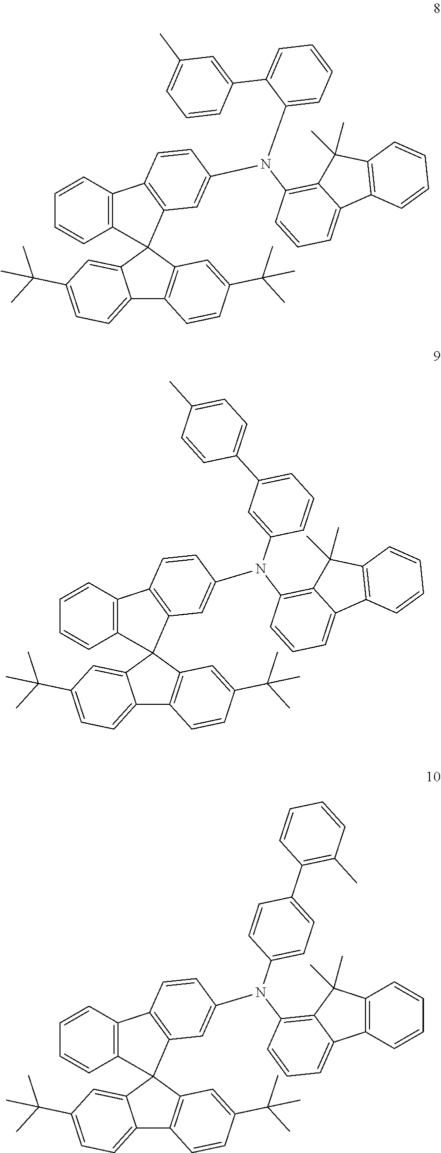 [SMILES: Cc1cccc(-c2ccccc2N(c2ccc3c(c2)C2(c4ccccc4-3)c3cc(C(C)(C)C)ccc3-c3ccc(C(C)(C)C)cc32)c2cccc3c2C(C)(C)c2ccccc2-3)c1 ; Cc1ccc(-c2cccc(N(c3ccc4c(c3)C3(c5ccccc5-4)c4cc(C(C)(C)C)ccc4-c4ccc(C(C)(C)C)cc43)c3cccc4c3C(C)(C)c3ccccc3-4)c2)cc1 ; Cc1ccccc1-c1ccc(N(c2ccc3c(c2)C2(c4ccccc4-3)c3cc(C(C)(C)C)ccc3-c3ccc(C(C)(C)C)cc32)c2cccc3c2C(C)(C)c2ccccc2-3)cc1]

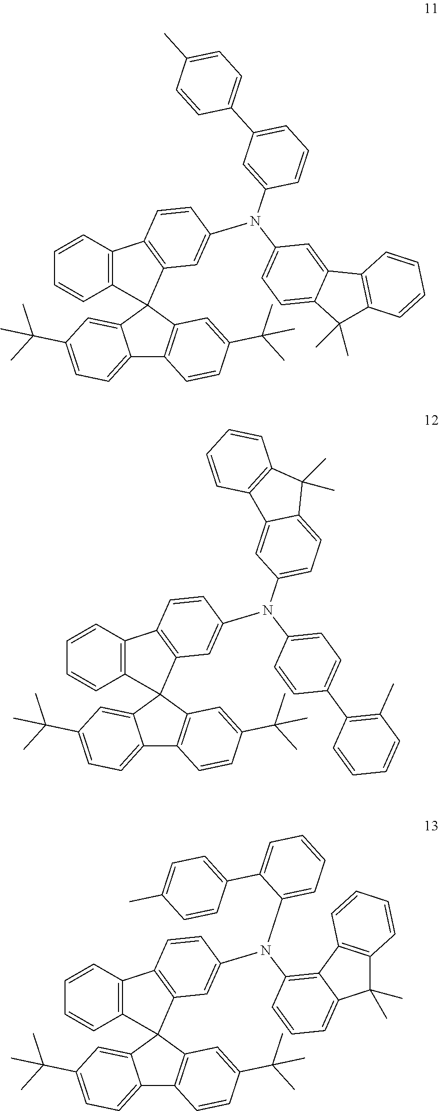 [SMILES: Cc1ccc(-c2cccc(N(c3ccc4c(c3)-c3ccccc3C4(C)C)c3ccc4c(c3)C3(c5ccccc5-4)c4cc(C(C)(C)C)ccc4-c4ccc(C(C)(C)C)cc43)c2)cc1 ; Cc1ccccc1-c1ccc(N(c2ccc3c(c2)-c2ccccc2C3(C)C)c2ccc3c(c2)C2(c4ccccc4-3)c3cc(C(C)(C)C)ccc3-c3ccc(C(C)(C)C)cc32)cc1 ; Cc1ccc(-c2ccccc2N(c2ccc3c(c2)C2(c4ccccc4-3)c3cc(C(C)(C)C)ccc3-c3ccc(C(C)(C)C)cc32)c2cccc3c2-c2ccccc2C3(C)C)cc1]

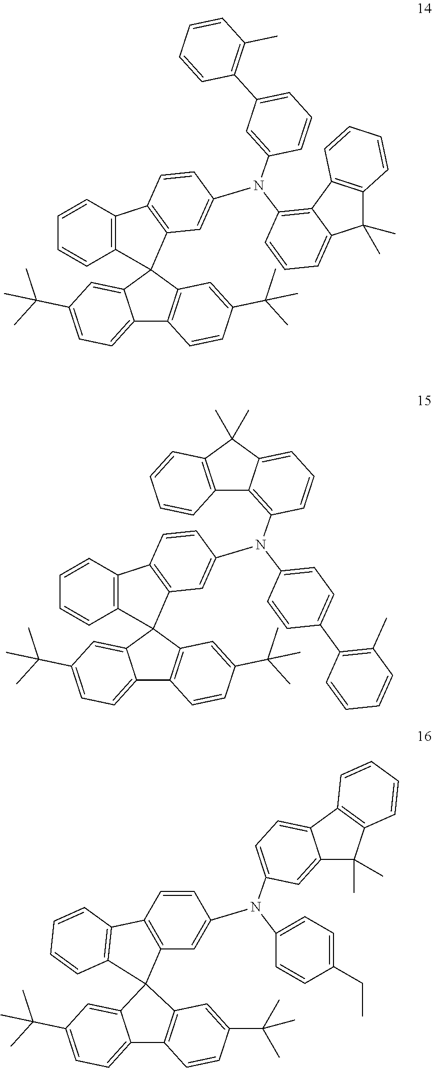

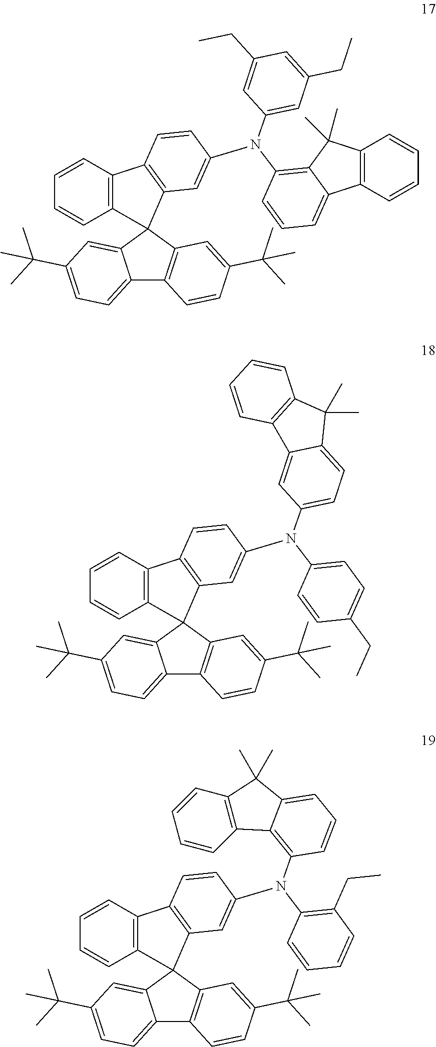 [SMILES: CCc1cc(CC)cc(N(c2ccc3c(c2)C2(c4ccccc4-3)c3cc(C(C)(C)C)ccc3-c3ccc(C(C)(C)C)cc32)c2cccc3c2C(C)(C)c2ccccc2-3)c1 ; CCc1ccc(N(c2ccc3c(c2)-c2ccccc2C3(C)C)c2ccc3c(c2)C2(c4ccccc4-3)c3cc(C(C)(C)C)ccc3-c3ccc(C(C)(C)C)cc32)cc1 ; CCc1ccccc1N(c1ccc2c(c1)C1(c3ccccc3-2)c2cc(C(C)(C)C)ccc2-c2ccc(C(C)(C)C)cc21)c1cccc2c1-c1ccccc1C2(C)C]

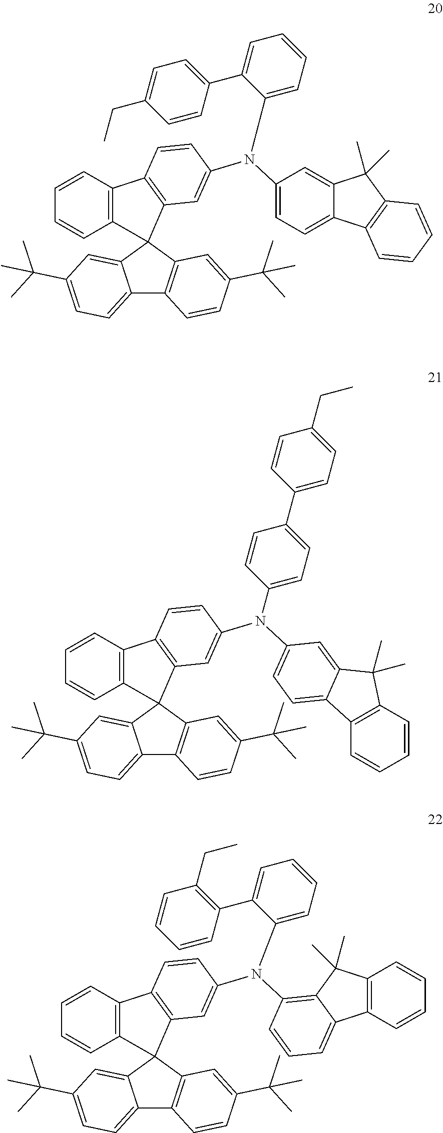 [SMILES: CCc1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1 ; CCc1ccc(-c2ccc(N(c3ccc4c(c3)C(C)(C)c3ccccc3-4)c3ccc4c(c3)C3(c5ccccc5-4)c4cc(C(C)(C)C)ccc4-c4ccc(C(C)(C)C)cc43)cc2)cc1 ; CCc1ccccc1-c1ccccc1N(c1ccc2c(c1)C1(c3ccccc3-2)c2cc(C(C)(C)C)ccc2-c2ccc(C(C)(C)C)cc21)c1cccc2c1C(C)(C)c1ccccc1-2]

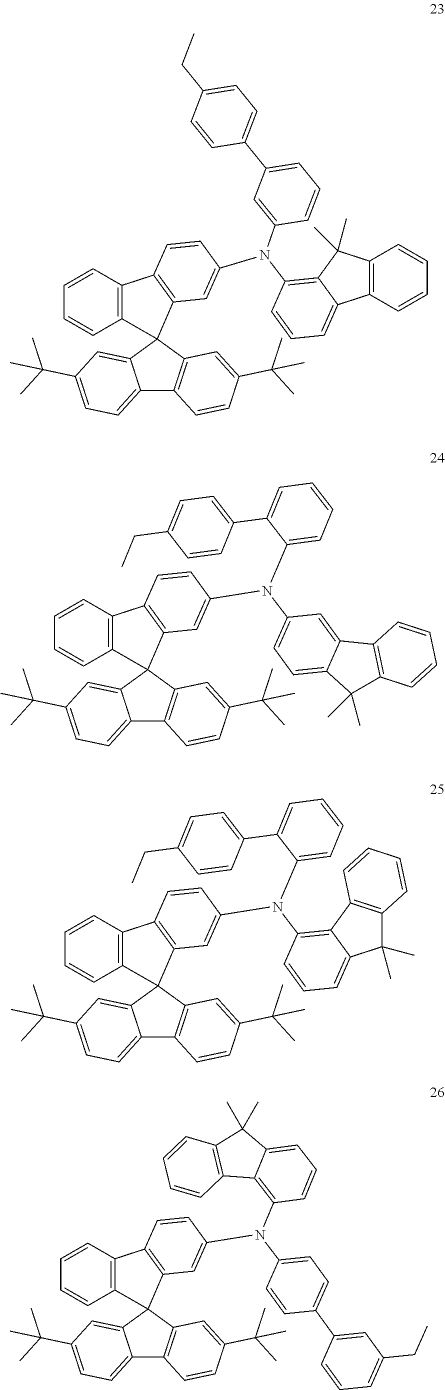

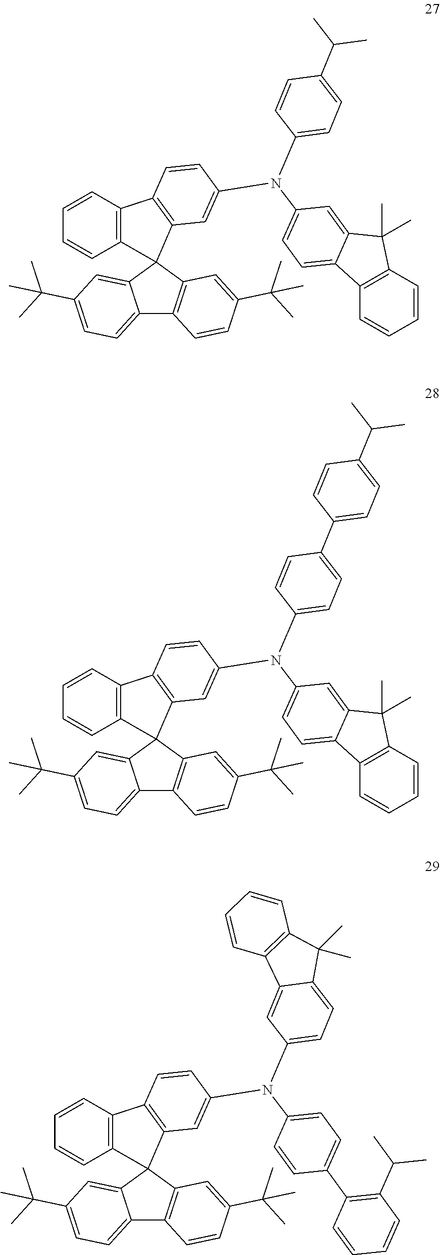 [SMILES: CC(C)c1ccc(N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1 ; CC(C)c1ccc(-c2ccc(N(c3ccc4c(c3)C(C)(C)c3ccccc3-4)c3ccc4c(c3)C3(c5ccccc5-4)c4cc(C(C)(C)C)ccc4-c4ccc(C(C)(C)C)cc43)cc2)cc1 ; CC(C)c1ccccc1-c1ccc(N(c2ccc3c(c2)-c2ccccc2C3(C)C)c2ccc3c(c2)C2(c4ccccc4-3)c3cc(C(C)(C)C)ccc3-c3ccc(C(C)(C)C)cc32)cc1]

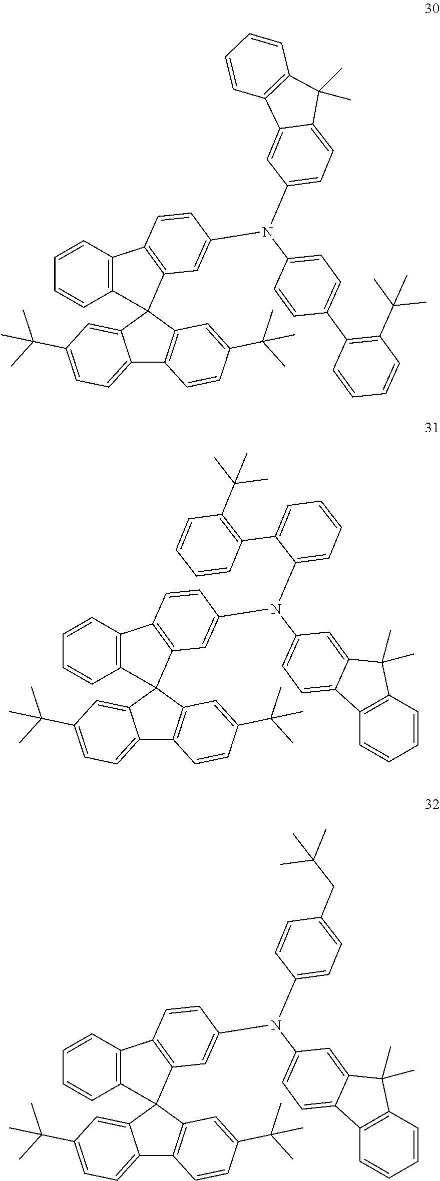

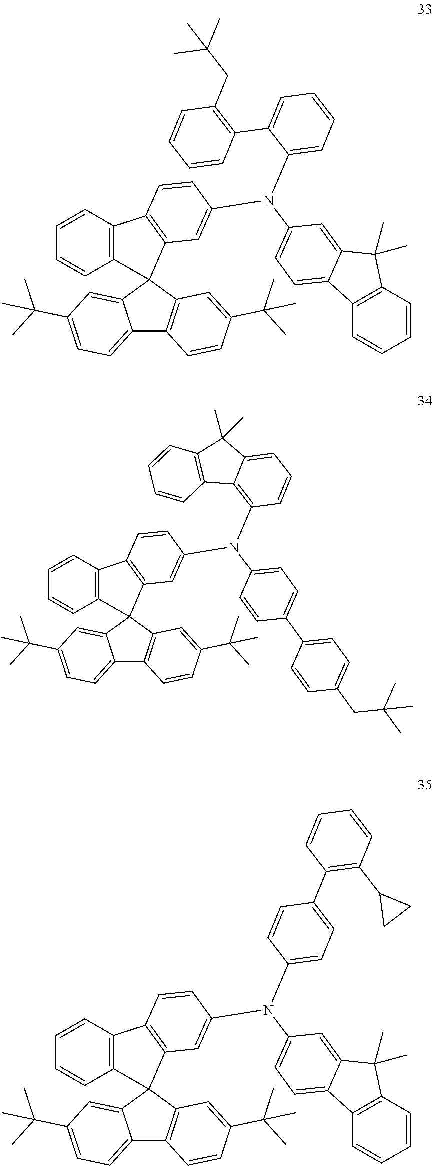

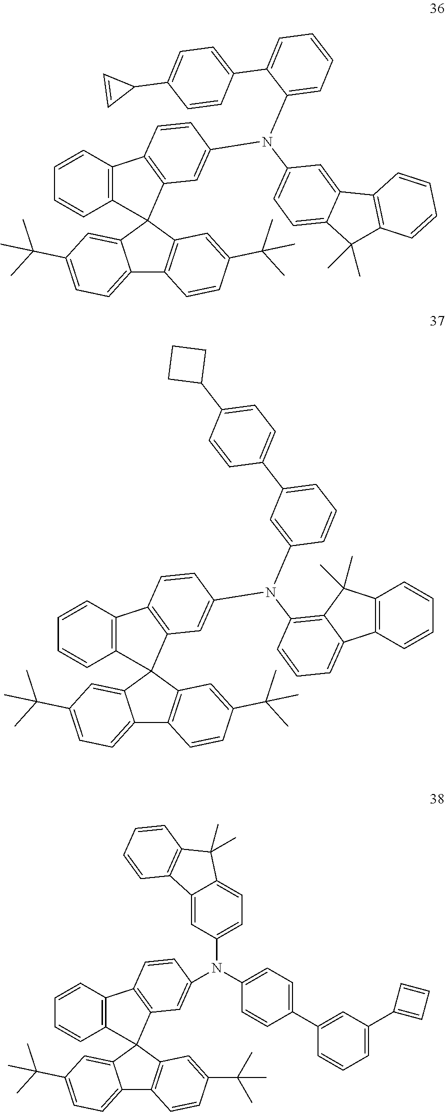 [SMILES: CC(C)(C)c1ccc2c(c1)C1(c3ccccc3-c3ccc(N(c4ccc5c(c4)-c4ccccc4C5(C)C)c4ccccc4-c4ccc(C5C=C5)cc4)cc31)c1cc(C(C)(C)C)ccc1-2 ; CC(C)(C)c1ccc2c(c1)C1(c3ccccc3-c3ccc(N(c4cccc(-c5ccc(C6CCC6)cc5)c4)c4cccc5c4C(C)(C)c4ccccc4-5)cc31)c1cc(C(C)(C)C)ccc1-2 ; CC(C)(C)c1ccc2c(c1)C1(c3ccccc3-c3ccc(N(c4ccc(-c5cccc(C6=CC=C6)c5)cc4)c4ccc5c(c4)-c4ccccc4C5(C)C)cc31)c1cc(C(C)(C)C)ccc1-2]

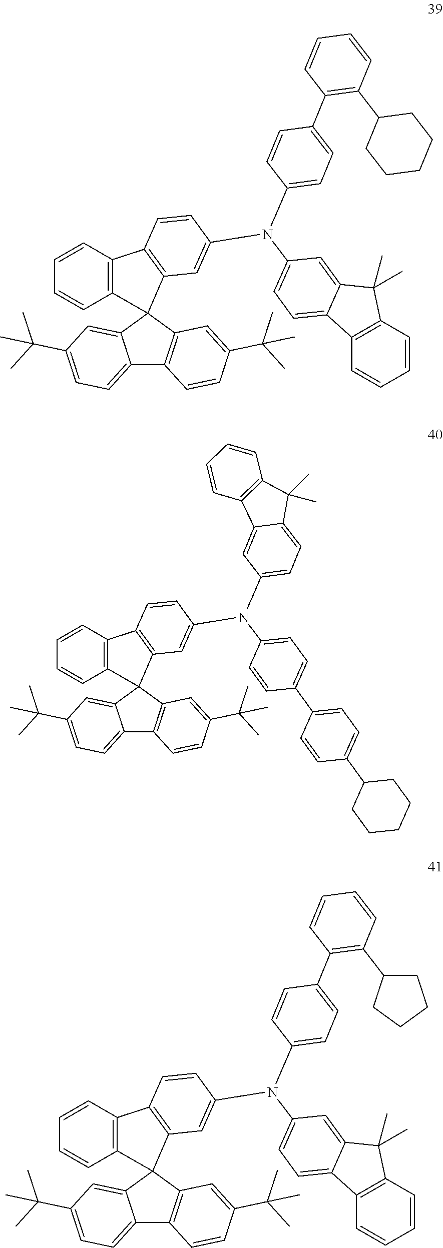 [SMILES: CC(C)(C)c1ccc2c(c1)C1(c3ccccc3-c3ccc(N(c4ccc(-c5ccccc5C5CCCCC5)cc4)c4ccc5c(c4)C(C)(C)c4ccccc4-5)cc31)c1cc(C(C)(C)C)ccc1-2 ; CC(C)(C)c1ccc2c(c1)C1(c3ccccc3-c3ccc(N(c4ccc(-c5ccc(C6CCCCC6)cc5)cc4)c4ccc5c(c4)-c4ccccc4C5(C)C)cc31)c1cc(C(C)(C)C)ccc1-2 ; CC(C)(C)c1ccc2c(c1)C1(c3ccccc3-c3ccc(N(c4ccc(-c5ccccc5C5CCCC5)cc4)c4ccc5c(c4)C(C)(C)c4ccccc4-5)cc31)c1cc(C(C)(C)C)ccc1-2]

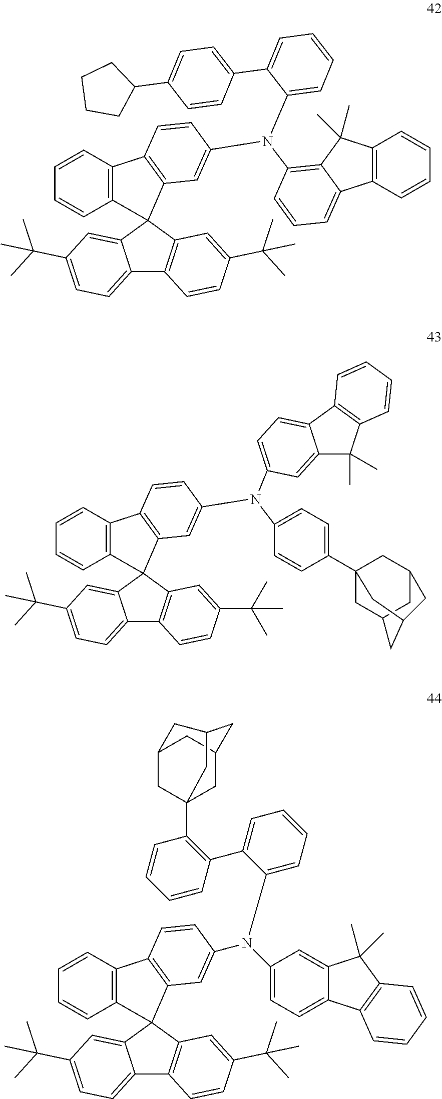 [SMILES: CC(C)(C)c1ccc2c(c1)C1(c3ccccc3-c3ccc(N(c4ccccc4-c4ccc(C5CCCC5)cc4)c4cccc5c4C(C)(C)c4ccccc4-5)cc31)c1cc(C(C)(C)C)ccc1-2 ; CC(C)(C)c1ccc2c(c1)C1(c3ccccc3-c3ccc(N(c4ccc(C56CC7CC(CC(C7)C5)C6)cc4)c4ccc5c(c4)C(C)(C)c4ccccc4-5)cc31)c1cc(C(C)(C)C)ccc1-2 ; CC(C)(C)c1ccc2c(c1)C1(c3ccccc3-c3ccc(N(c4ccc5c(c4)C(C)(C)c4ccccc4-5)c4ccccc4-c4ccccc4C45CC6CC(CC(C6)C4)C5)cc31)c1cc(C(C)(C)C)ccc1-2]

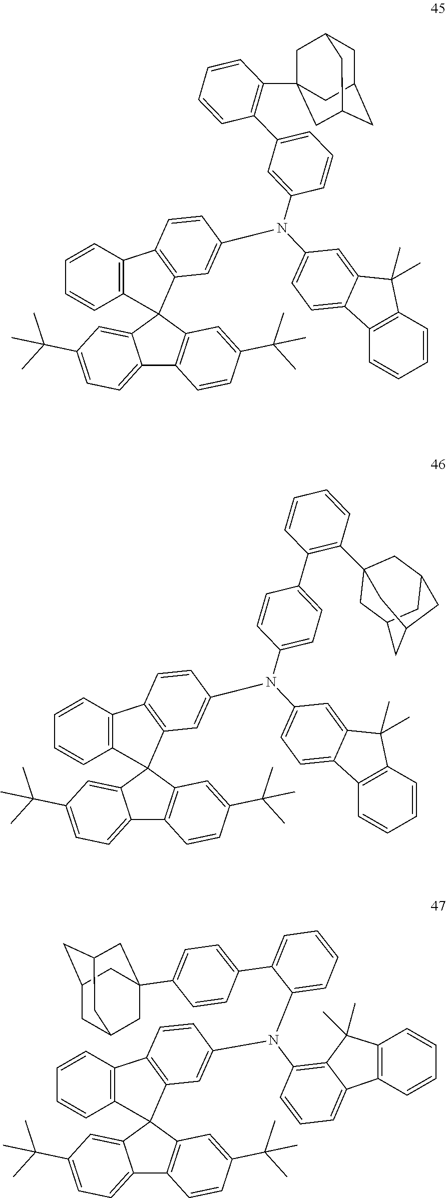 [SMILES: CC(C)(C)c1ccc2c(c1)C1(c3ccccc3-c3ccc(N(c4cccc(-c5ccccc5C56CC7CC(CC(C7)C5)C6)c4)c4ccc5c(c4)C(C)(C)c4ccccc4-5)cc31)c1cc(C(C)(C)C)ccc1-2 ; CC(C)(C)c1ccc2c(c1)C1(c3ccccc3-c3ccc(N(c4ccc(-c5ccccc5C56CC7CC(CC(C7)C5)C6)cc4)c4ccc5c(c4)C(C)(C)c4ccccc4-5)cc31)c1cc(C(C)(C)C)ccc1-2 ; CC(C)(C)c1ccc2c(c1)C1(c3ccccc3-c3ccc(N(c4ccccc4-c4ccc(C56CC7CC(CC(C7)C5)C6)cc4)c4cccc5c4C(C)(C)c4ccccc4-5)cc31)c1cc(C(C)(C)C)ccc1-2]

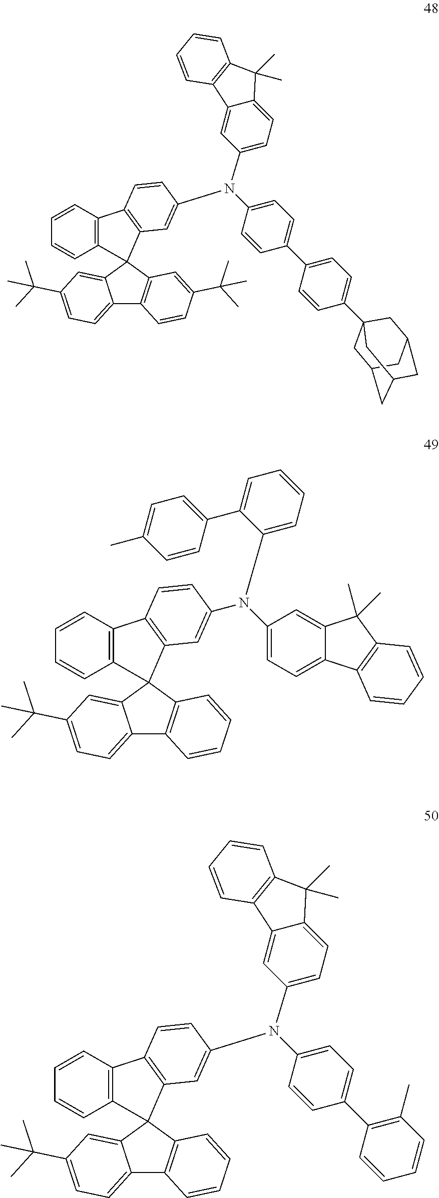 [SMILES: CC(C)(C)c1ccc2c(c1)C1(c3ccccc3-c3ccc(N(c4ccc(-c5ccc(C67CC8CC(CC(C8)C6)C7)cc5)cc4)c4ccc5c(c4)-c4ccccc4C5(C)C)cc31)c1cc(C(C)(C)C)ccc1-2 ; Cc1ccc(-c2ccccc2N(c2ccc3c(c2)C(C)(C)c2ccccc2-3)c2ccc3c(c2)C2(c4ccccc4-3)c3ccccc3-c3ccc(C(C)(C)C)cc32)cc1 ; Cc1ccccc1-c1ccc(N(c2ccc3c(c2)-c2ccccc2C3(C)C)c2ccc3c(c2)C2(c4ccccc4-3)c3ccccc3-c3ccc(C(C)(C)C)cc32)cc1]

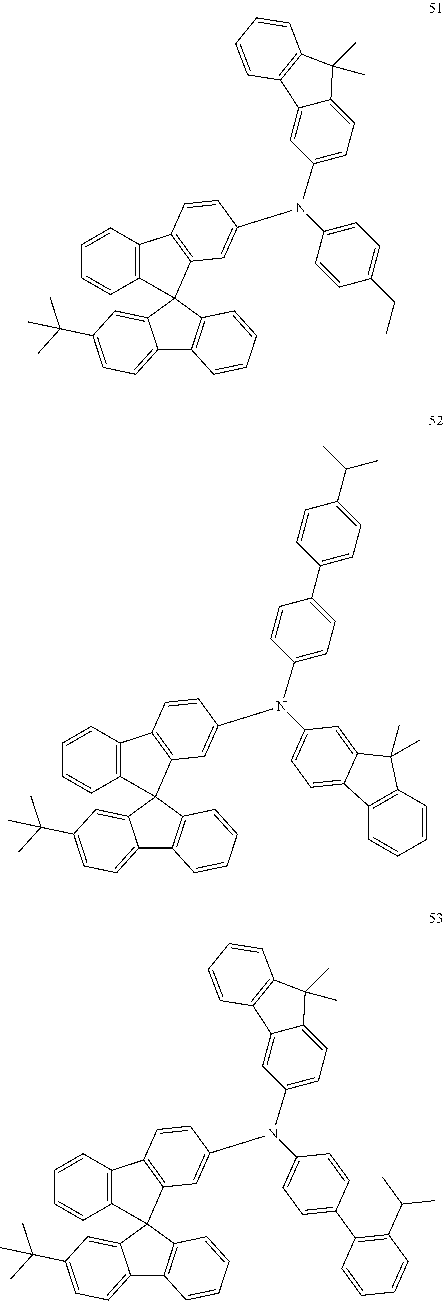 [SMILES: CCc1ccc(N(c2ccc3c(c2)-c2ccccc2C3(C)C)c2ccc3c(c2)C2(c4ccccc4-3)c3ccccc3-c3ccc(C(C)(C)C)cc32)cc1 ; CC(C)c1ccc(-c2ccc(N(c3ccc4c(c3)C(C)(C)c3ccccc3-4)c3ccc4c(c3)C3(c5ccccc5-4)c4ccccc4-c4ccc(C(C)(C)C)cc43)cc2)cc1 ; CC(C)c1ccccc1-c1ccc(N(c2ccc3c(c2)-c2ccccc2C3(C)C)c2ccc3c(c2)C2(c4ccccc4-3)c3ccccc3-c3ccc(C(C)(C)C)cc32)cc1]

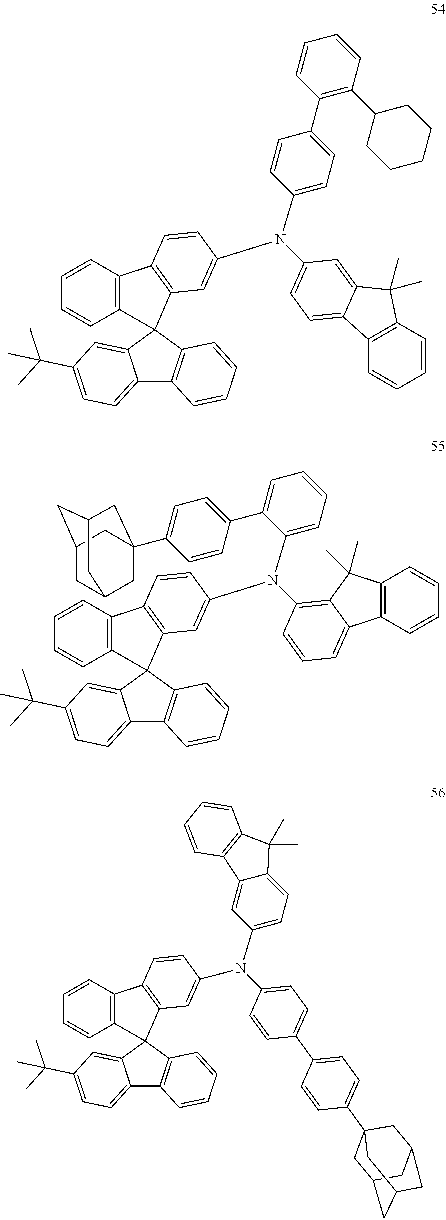 [SMILES: CC(C)(C)c1ccc2c(c1)C1(c3ccccc3-c3ccc(N(c4ccc(-c5ccccc5C5CCCCC5)cc4)c4ccc5c(c4)C(C)(C)c4ccccc4-5)cc31)c1ccccc1-2 ; CC(C)(C)c1ccc2c(c1)C1(c3ccccc3-c3ccc(N(c4ccccc4-c4ccc(C56CC7CC(CC(C7)C5)C6)cc4)c4cccc5c4C(C)(C)c4ccccc4-5)cc31)c1ccccc1-2 ; CC(C)(C)c1ccc2c(c1)C1(c3ccccc3-c3ccc(N(c4ccc(-c5ccc(C67CC8CC(CC(C8)C6)C7)cc5)cc4)c4ccc5c(c4)-c4ccccc4C5(C)C)cc31)c1ccccc1-2]

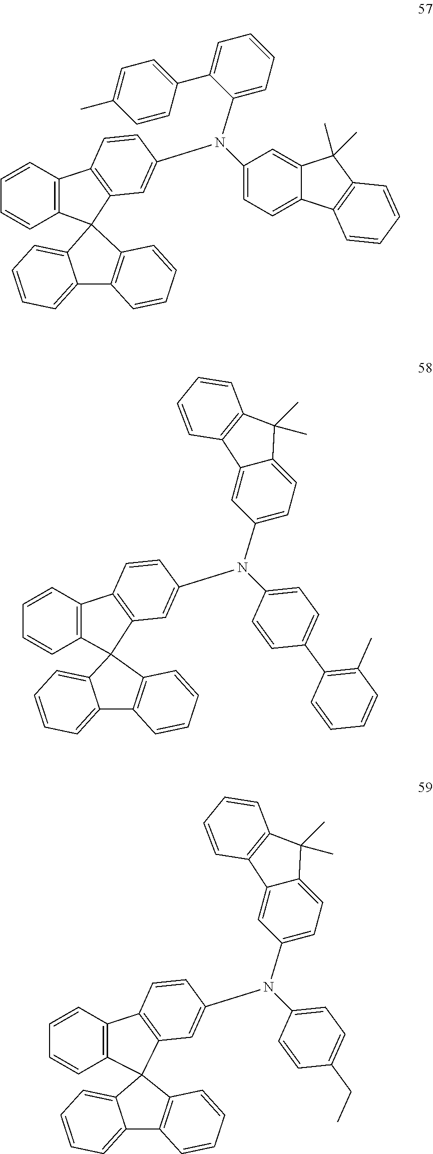 [SMILES: Cc1ccc(-c2ccccc2N(c2ccc3c(c2)C(C)(C)c2ccccc2-3)c2ccc3c(c2)C2(c4ccccc4-c4ccccc42)c2ccccc2-3)cc1 ; Cc1ccccc1-c1ccc(N(c2ccc3c(c2)-c2ccccc2C3(C)C)c2ccc3c(c2)C2(c4ccccc4-c4ccccc42)c2ccccc2-3)cc1 ; CCc1ccc(N(c2ccc3c(c2)-c2ccccc2C3(C)C)c2ccc3c(c2)C2(c4ccccc4-c4ccccc42)c2ccccc2-3)cc1]

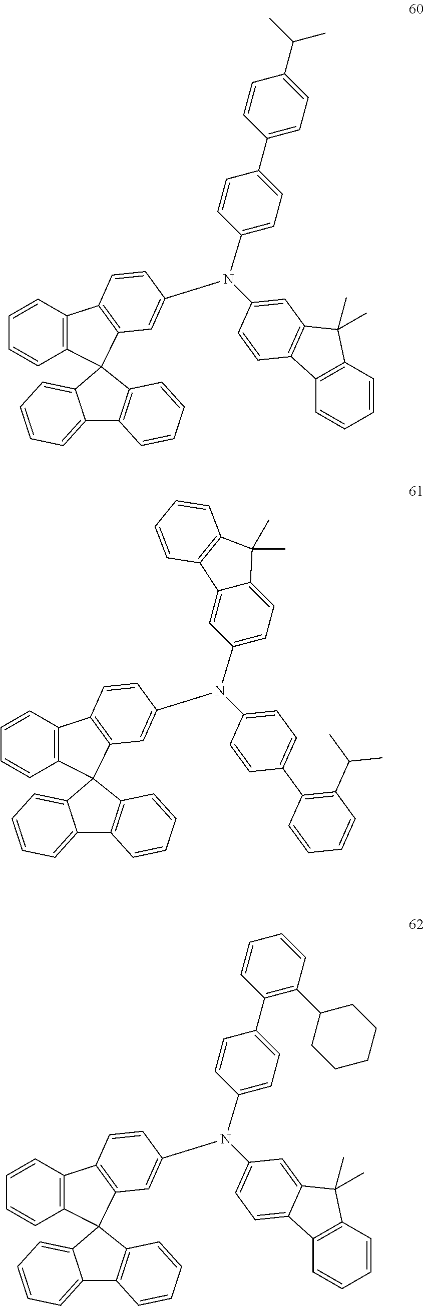 [SMILES: CC(C)c1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; CC(C)c1ccccc1-c1ccc(N(c2ccc3c(c2)-c2ccccc2C3(C)C)c2ccc3c(c2)C2(c4ccccc4-c4ccccc42)c2ccccc2-3)cc1 ; CC1(C)c2ccccc2-c2ccc(N(c3ccc(-c4ccccc4C4CCCCC4)cc3)c3ccc4c(c3)C3(c5ccccc5-c5ccccc53)c3ccccc3-4)cc21]

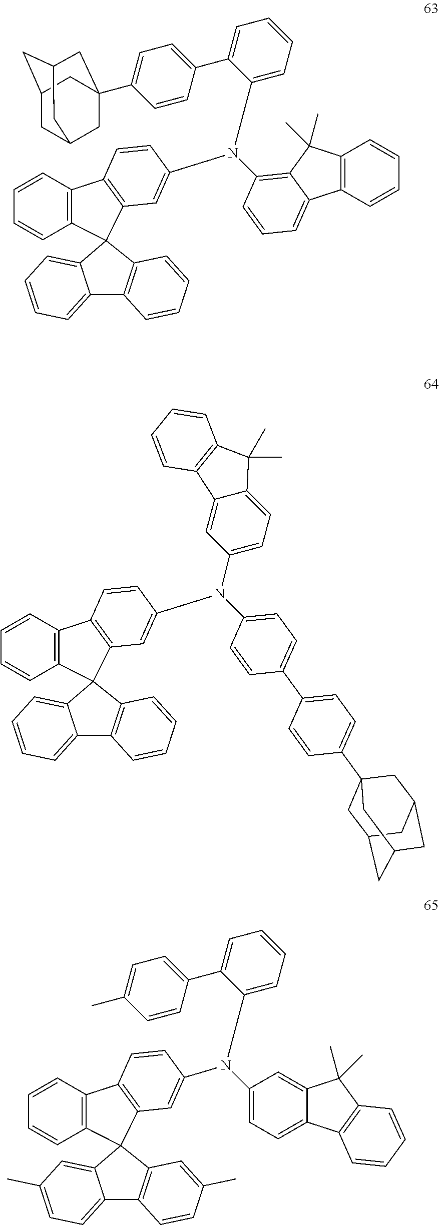 [SMILES: CC1(C)c2ccccc2-c2cccc(N(c3ccc4c(c3)C3(c5ccccc5-c5ccccc53)c3ccccc3-4)c3ccccc3-c3ccc(C45CC6CC(CC(C6)C4)C5)cc3)c21 ; CC1(C)c2ccccc2-c2cc(N(c3ccc(-c4ccc(C56CC7CC(CC(C7)C5)C6)cc4)cc3)c3ccc4c(c3)C3(c5ccccc5-c5ccccc53)c3ccccc3-4)ccc21 ; Cc1ccc(-c2ccccc2N(c2ccc3c(c2)C(C)(C)c2ccccc2-3)c2ccc3c(c2)C2(c4ccccc4-3)c3cc(C)ccc3-c3ccc(C)cc32)cc1]

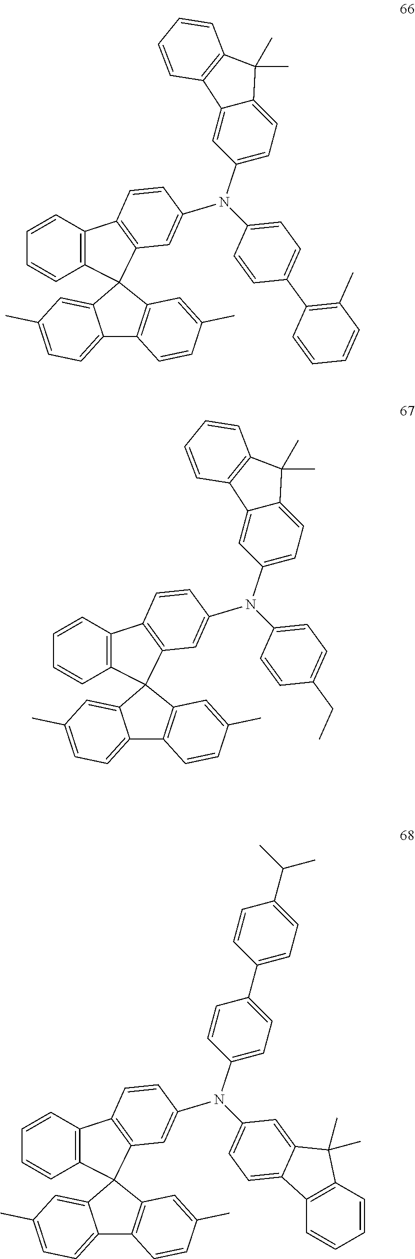 [SMILES: Cc1ccc2c(c1)C1(c3ccccc3-c3ccc(N(c4ccc(-c5ccccc5C)cc4)c4ccc5c(c4)-c4ccccc4C5(C)C)cc31)c1cc(C)ccc1-2 ; CCc1ccc(N(c2ccc3c(c2)-c2ccccc2C3(C)C)c2ccc3c(c2)C2(c4ccccc4-3)c3cc(C)ccc3-c3ccc(C)cc32)cc1 ; Cc1ccc2c(c1)C1(c3ccccc3-c3ccc(N(c4ccc(-c5ccc(C(C)C)cc5)cc4)c4ccc5c(c4)C(C)(C)c4ccccc4-5)cc31)c1cc(C)ccc1-2]

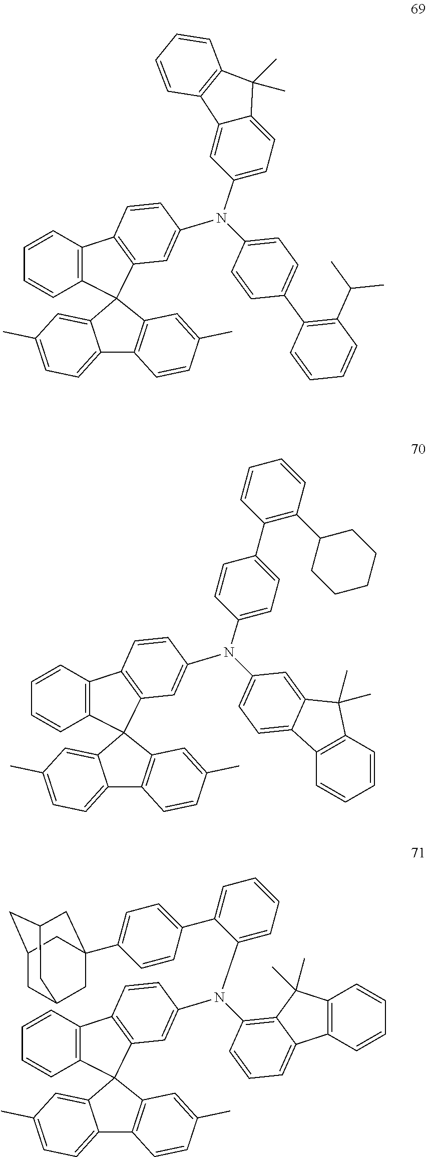

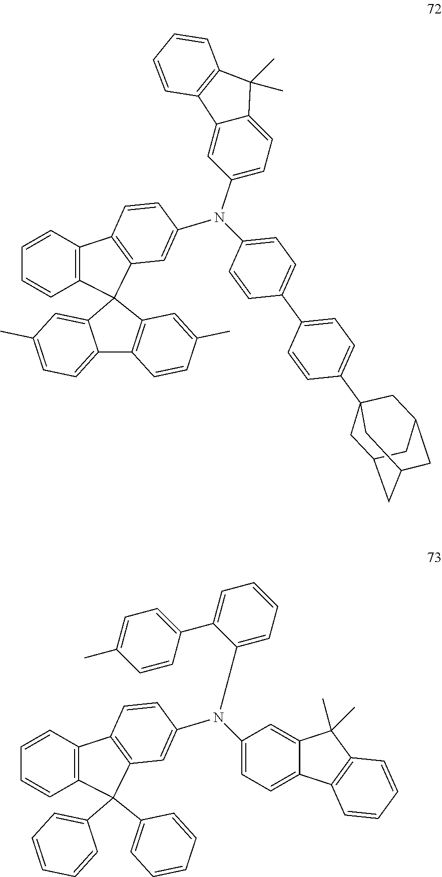 [SMILES: Cc1ccc2c(c1)C1(c3ccccc3-c3ccc(N(c4ccc(-c5ccc(C67CC8CC(CC(C8)C6)C7)cc5)cc4)c4ccc5c(c4)-c4ccccc4C5(C)C)cc31)c1cc(C)ccc1-2 ; Cc1ccc(-c2ccccc2N(c2ccc3c(c2)C(C)(C)c2ccccc2-3)c2ccc3c(c2)C(c2ccccc2)(c2ccccc2)c2ccccc2-3)cc1]

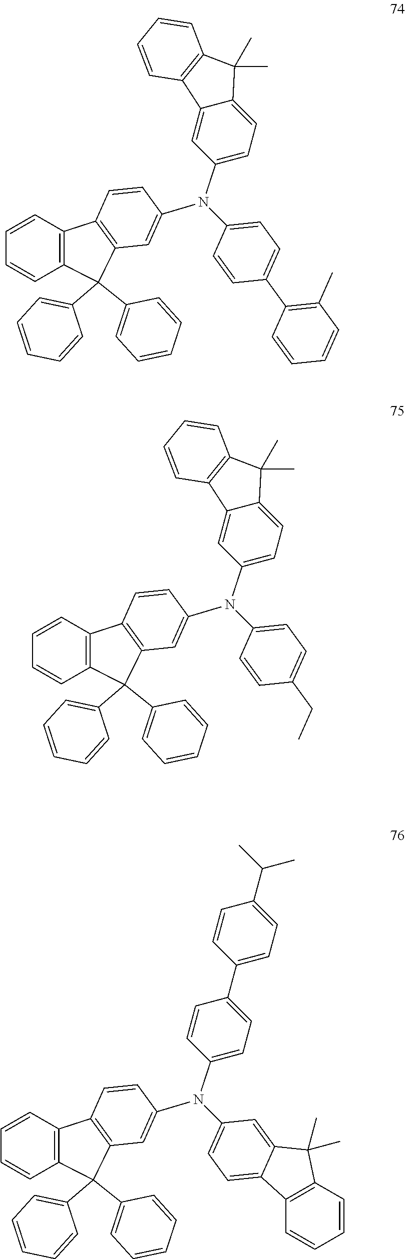 [SMILES: Cc1ccccc1-c1ccc(N(c2ccc3c(c2)-c2ccccc2C3(C)C)c2ccc3c(c2)C(c2ccccc2)(c2ccccc2)c2ccccc2-3)cc1 ; CCc1ccc(N(c2ccc3c(c2)-c2ccccc2C3(C)C)c2ccc3c(c2)C(c2ccccc2)(c2ccccc2)c2ccccc2-3)cc1 ; CC(C)c1ccc(-c2ccc(N(c3ccc4c(c3)C(C)(C)c3ccccc3-4)c3ccc4c(c3)C(c3ccccc3)(c3ccccc3)c3ccccc3-4)cc2)cc1]

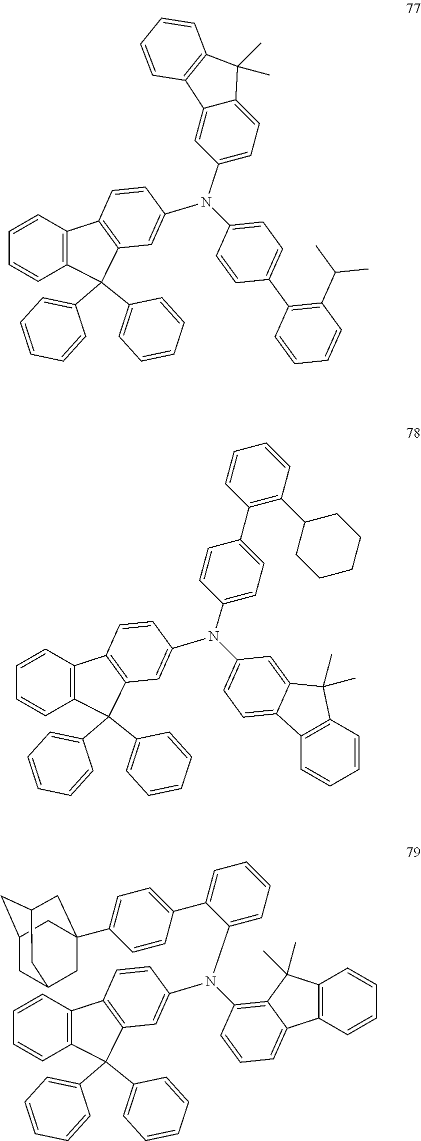 [SMILES: CC(C)c1ccccc1-c1ccc(N(c2ccc3c(c2)-c2ccccc2C3(C)C)c2ccc3c(c2)C(c2ccccc2)(c2ccccc2)c2ccccc2-3)cc1 ; CC1(C)c2ccccc2-c2ccc(N(c3ccc(-c4ccccc4C4CCCCC4)cc3)c3ccc4c(c3)C(c3ccccc3)(c3ccccc3)c3ccccc3-4)cc21 ; CC1(C)c2ccccc2-c2cccc(N(c3ccc4c(c3)C(c3ccccc3)(c3ccccc3)c3ccccc3-4)c3ccccc3-c3ccc(C45CC6CC(CC(C6)C4)C5)cc3)c21]

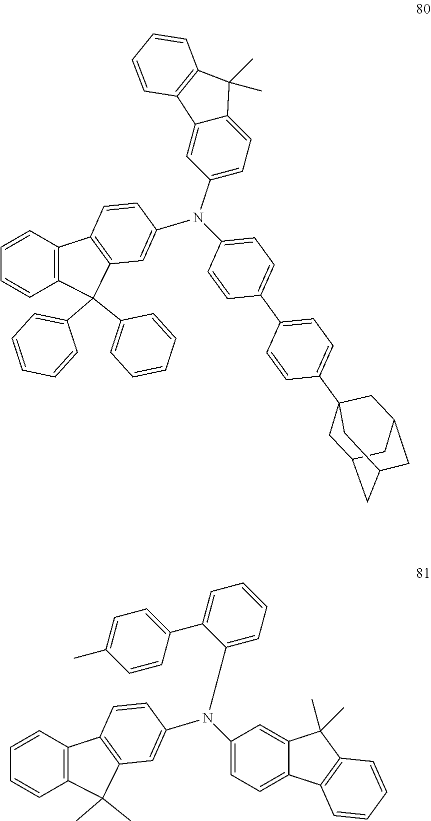 [SMILES: CC1(C)c2ccccc2-c2cc(N(c3ccc(-c4ccc(C56CC7CC(CC(C7)C5)C6)cc4)cc3)c3ccc4c(c3)C(c3ccccc3)(c3ccccc3)c3ccccc3-4)ccc21 ; Cc1ccc(-c2ccccc2N(c2ccc3c(c2)C(C)(C)c2ccccc2-3)c2ccc3c(c2)C(C)(C)c2ccccc2-3)cc1]

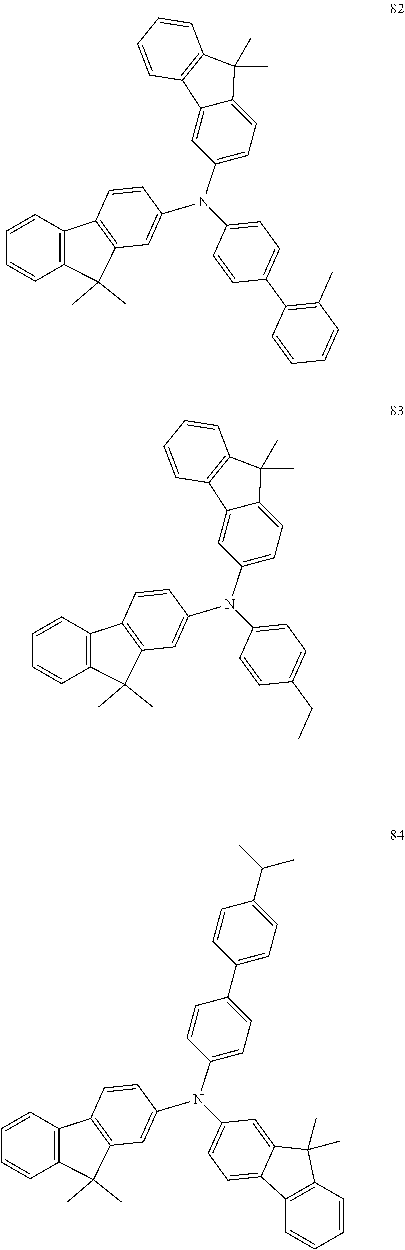 [SMILES: Cc1ccccc1-c1ccc(N(c2ccc3c(c2)-c2ccccc2C3(C)C)c2ccc3c(c2)C(C)(C)c2ccccc2-3)cc1 ; CCc1ccc(N(c2ccc3c(c2)-c2ccccc2C3(C)C)c2ccc3c(c2)C(C)(C)c2ccccc2-3)cc1 ; CC(C)c1ccc(-c2ccc(N(c3ccc4c(c3)C(C)(C)c3ccccc3-4)c3ccc4c(c3)C(C)(C)c3ccccc3-4)cc2)cc1]

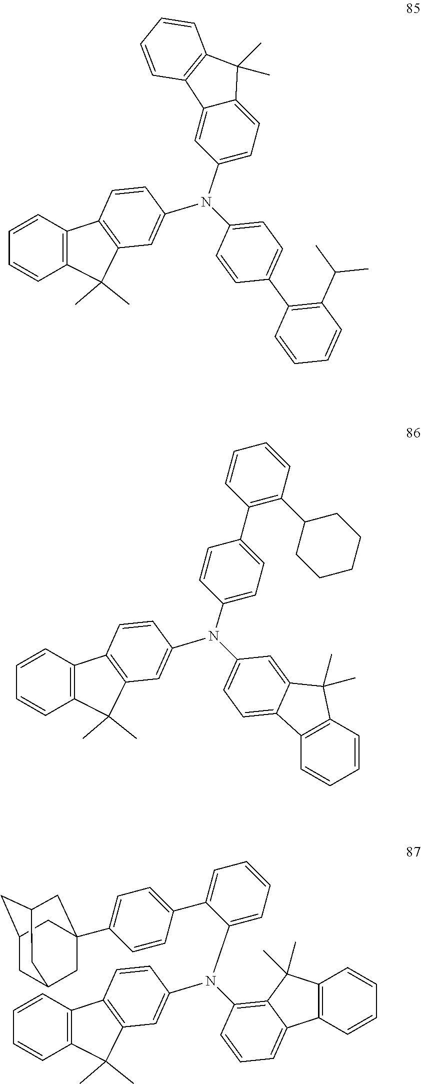 [SMILES: CC(C)c1ccccc1-c1ccc(N(c2ccc3c(c2)-c2ccccc2C3(C)C)c2ccc3c(c2)C(C)(C)c2ccccc2-3)cc1 ; CC1(C)c2ccccc2-c2ccc(N(c3ccc(-c4ccccc4C4CCCCC4)cc3)c3ccc4c(c3)C(C)(C)c3ccccc3-4)cc21 ; CC1(C)c2ccccc2-c2ccc(N(c3ccccc3-c3ccc(C45CC6CC(CC(C6)C4)C5)cc3)c3cccc4c3C(C)(C)c3ccccc3-4)cc21]

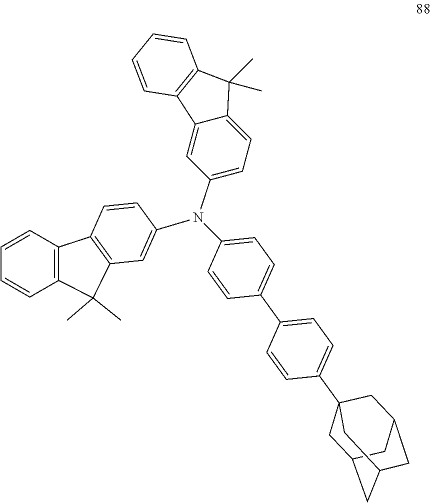 [SMILES: CC1(C)c2ccccc2-c2cc(N(c3ccc(-c4ccc(C56CC7CC(CC(C7)C5)C6)cc4)cc3)c3ccc4c(c3)C(C)(C)c3ccccc3-4)ccc21]

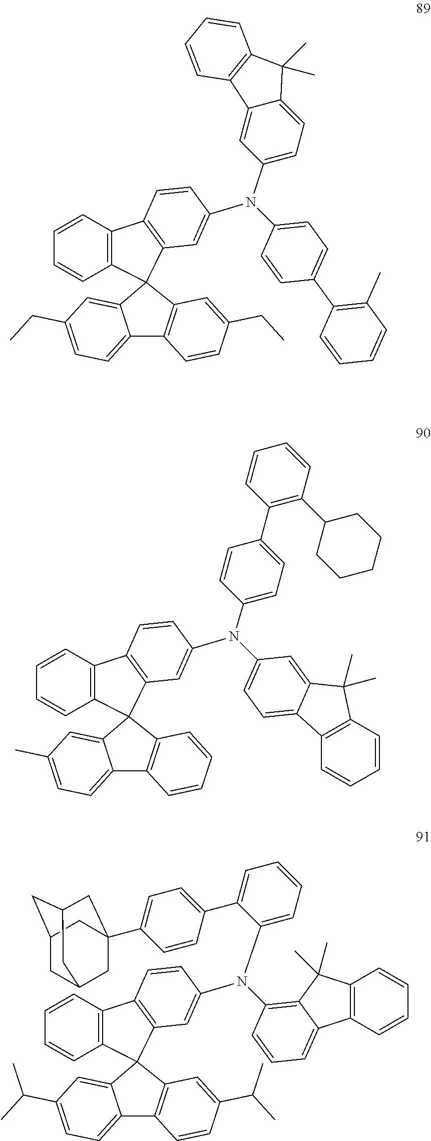 [SMILES: CCc1ccc2c(c1)C1(c3ccccc3-c3ccc(N(c4ccc(-c5ccccc5C)cc4)c4ccc5c(c4)-c4ccccc4C5(C)C)cc31)c1cc(CC)ccc1-2 ; Cc1ccc2c(c1)C1(c3ccccc3-2)c2ccccc2-c2ccc(N(c3ccc(-c4ccccc4C4CCCCC4)cc3)c3ccc4c(c3)C(C)(C)c3ccccc3-4)cc21 ; CC(C)c1ccc2c(c1)C1(c3ccccc3-c3ccc(N(c4ccccc4-c4ccc(C56CC7CC(CC(C7)C5)C6)cc4)c4cccc5c4C(C)(C)c4ccccc4-5)cc31)c1cc(C(C)C)ccc1-2]

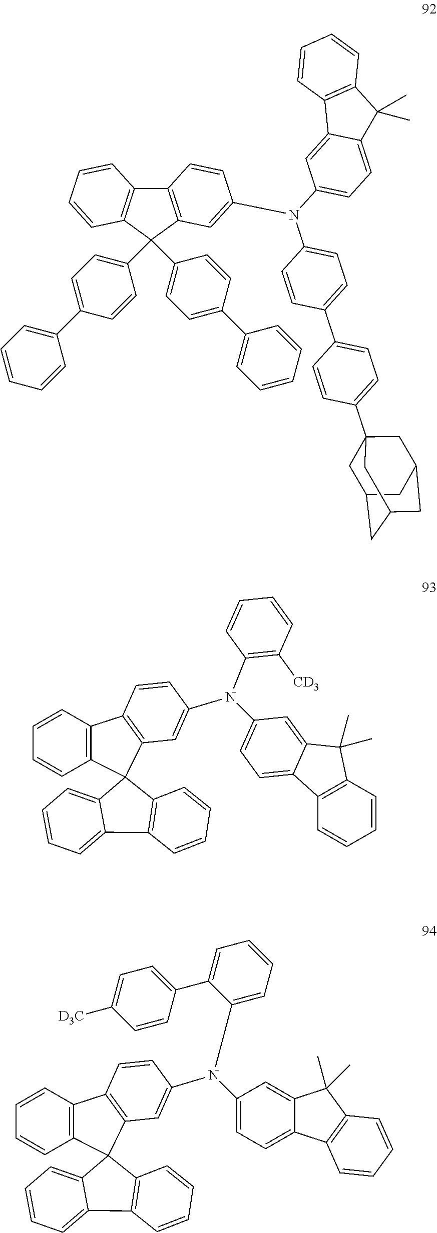 [SMILES: CC1(C)c2ccccc2-c2cc(N(c3ccc(-c4ccc(C56CC7CC(CC(C7)C5)C6)cc4)cc3)c3ccc4c(c3)C(c3ccc(-c5ccccc5)cc3)(c3ccc(-c5ccccc5)cc3)c3ccccc3-4)ccc21 ; [2H]C([2H])([2H])c1ccccc1N(c1ccc2c(c1)C(C)(C)c1ccccc1-2)c1ccc2c(c1)C1(c3ccccc3-c3ccccc31)c1ccccc1-2 ; [2H]C([2H])([2H])c1ccc(-c2ccccc2N(c2ccc3c(c2)C(C)(C)c2ccccc2-3)c2ccc3c(c2)C2(c4ccccc4-c4ccccc42)c2ccccc2-3)cc1]

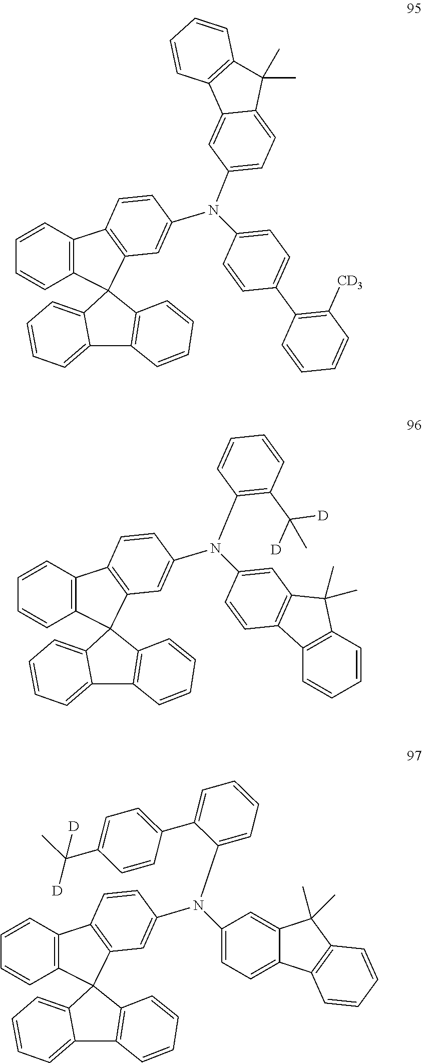 [SMILES: [2H]C([2H])([2H])c1ccccc1-c1ccc(N(c2ccc3c(c2)-c2ccccc2C3(C)C)c2ccc3c(c2)C2(c4ccccc4-c4ccccc42)c2ccccc2-3)cc1 ; [2H]C([2H])(C)c1ccccc1N(c1ccc2c(c1)C(C)(C)c1ccccc1-2)c1ccc2c(c1)C1(c3ccccc3-c3ccccc31)c1ccccc1-2 ; [2H]C([2H])(C)c1ccc(-c2ccccc2N(c2ccc3c(c2)C(C)(C)c2ccccc2-3)c2ccc3c(c2)C2(c4ccccc4-c4ccccc42)c2ccccc2-3)cc1]

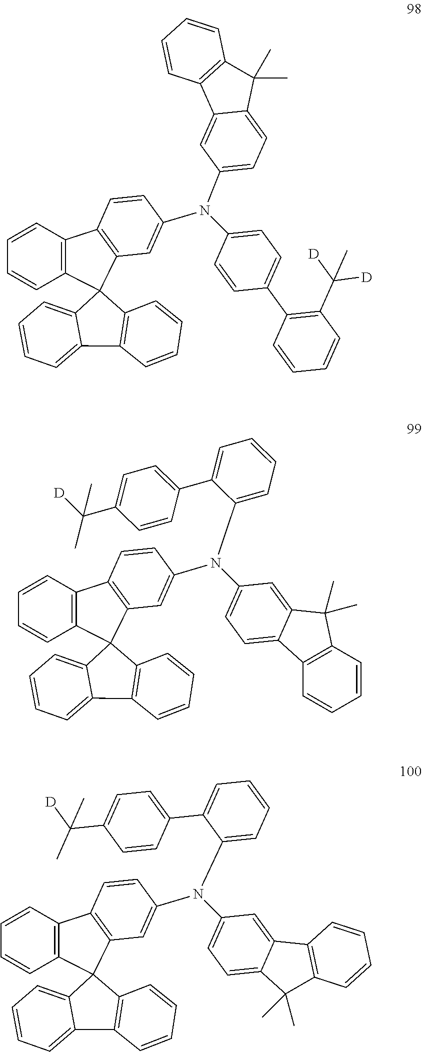 [SMILES: [2H]C([2H])(C)c1ccccc1-c1ccc(N(c2ccc3c(c2)-c2ccccc2C3(C)C)c2ccc3c(c2)C2(c4ccccc4-c4ccccc42)c2ccccc2-3)cc1 ; [2H]C(C)(C)c1ccc(-c2ccccc2N(c2ccc3c(c2)C(C)(C)c2ccccc2-3)c2ccc3c(c2)C2(c4ccccc4-c4ccccc42)c2ccccc2-3)cc1 ; [2H]C(C)(C)c1ccc(-c2ccccc2N(c2ccc3c(c2)-c2ccccc2C3(C)C)c2ccc3c(c2)C2(c4ccccc4-c4ccccc42)c2ccccc2-3)cc1]

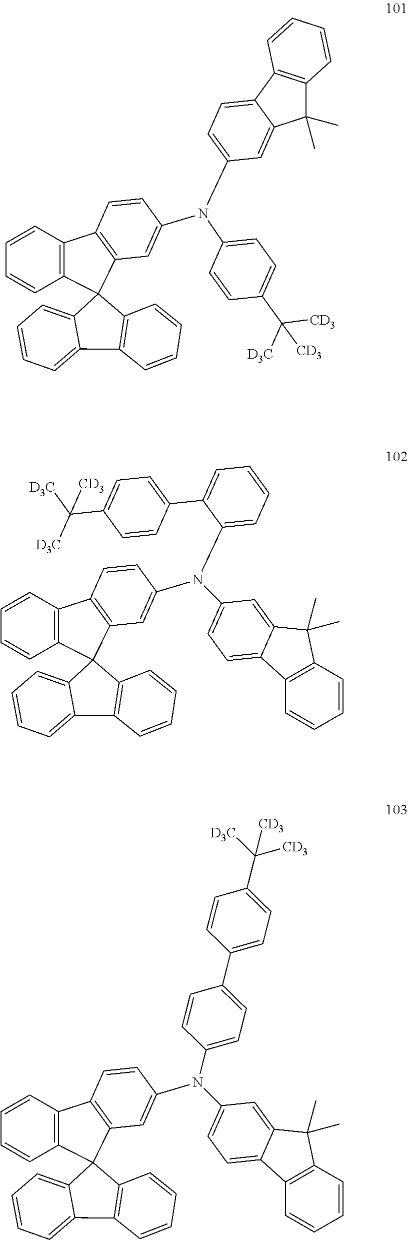 [SMILES: [2H]C([2H])([2H])C(c1ccc(N(c2ccc3c(c2)C(C)(C)c2ccccc2-3)c2ccc3c(c2)C2(c4ccccc4-c4ccccc42)c2ccccc2-3)cc1)(C([2H])([2H])[2H])C([2H])([2H])[2H] ; [2H]C([2H])([2H])C(c1ccc(-c2ccccc2N(c2ccc3c(c2)C(C)(C)c2ccccc2-3)c2ccc3c(c2)C2(c4ccccc4-c4ccccc42)c2ccccc2-3)cc1)(C([2H])([2H])[2H])C([2H])([2H])[2H] ; [2H]C([2H])([2H])C(c1ccc(-c2ccc(N(c3ccc4c(c3)C(C)(C)c3ccccc3-4)c3ccc4c(c3)C3(c5ccccc5-c5ccccc53)c3ccccc3-4)cc2)cc1)(C([2H])([2H])[2H])C([2H])([2H])[2H]]

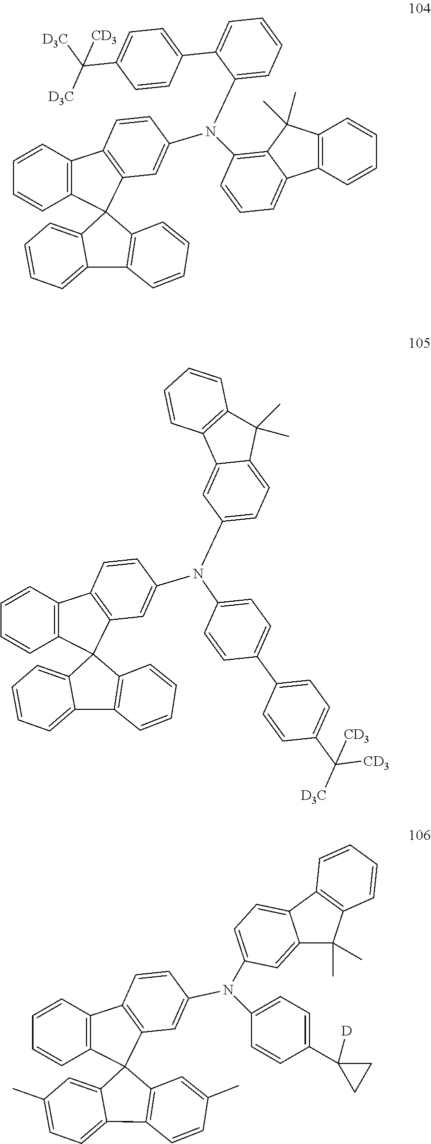 [SMILES: [2H]C([2H])([2H])C(c1ccc(-c2ccccc2N(c2ccc3c(c2)C2(c4ccccc4-c4ccccc42)c2ccccc2-3)c2cccc3c2C(C)(C)c2ccccc2-3)cc1)(C([2H])([2H])[2H])C([2H])([2H])[2H] ; [2H]C([2H])([2H])C(c1ccc(-c2ccc(N(c3ccc4c(c3)-c3ccccc3C4(C)C)c3ccc4c(c3)C3(c5ccccc5-c5ccccc53)c3ccccc3-4)cc2)cc1)(C([2H])([2H])[2H])C([2H])([2H])[2H] ; [2H]C1(c2ccc(N(c3ccc4c(c3)C(C)(C)c3ccccc3-4)c3ccc4c(c3)C3(c5ccccc5-4)c4cc(C)ccc4-c4ccc(C)cc43)cc2)CC1]

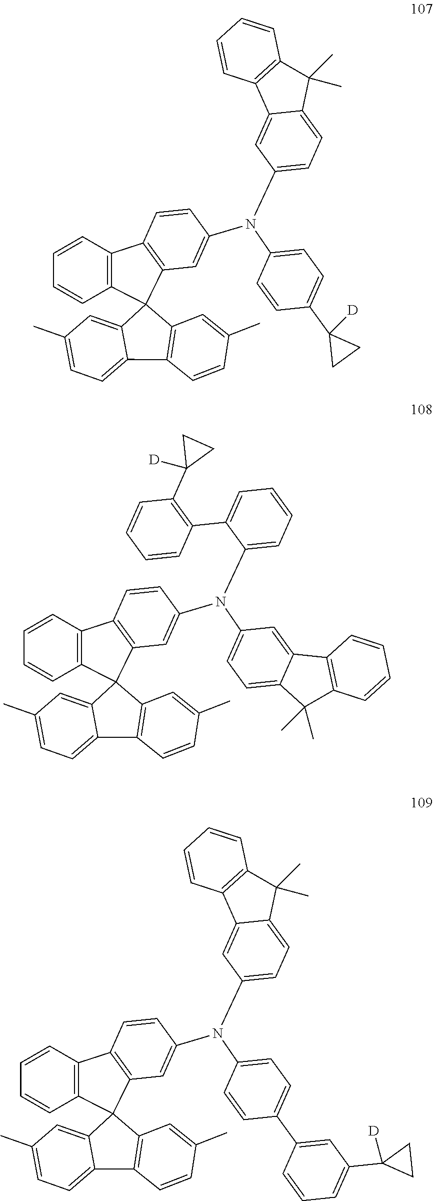 [SMILES: [2H]C1(c2ccc(N(c3ccc4c(c3)-c3ccccc3C4(C)C)c3ccc4c(c3)C3(c5ccccc5-4)c4cc(C)ccc4-c4ccc(C)cc43)cc2)CC1 ; [2H]C1(c2ccccc2-c2ccccc2N(c2ccc3c(c2)-c2ccccc2C3(C)C)c2ccc3c(c2)C2(c4ccccc4-3)c3cc(C)ccc3-c3ccc(C)cc32)CC1 ; [2H]C1(c2cccc(-c3ccc(N(c4ccc5c(c4)-c4ccccc4C5(C)C)c4ccc5c(c4)C4(c6ccccc6-5)c5cc(C)ccc5-c5ccc(C)cc54)cc3)c2)CC1]

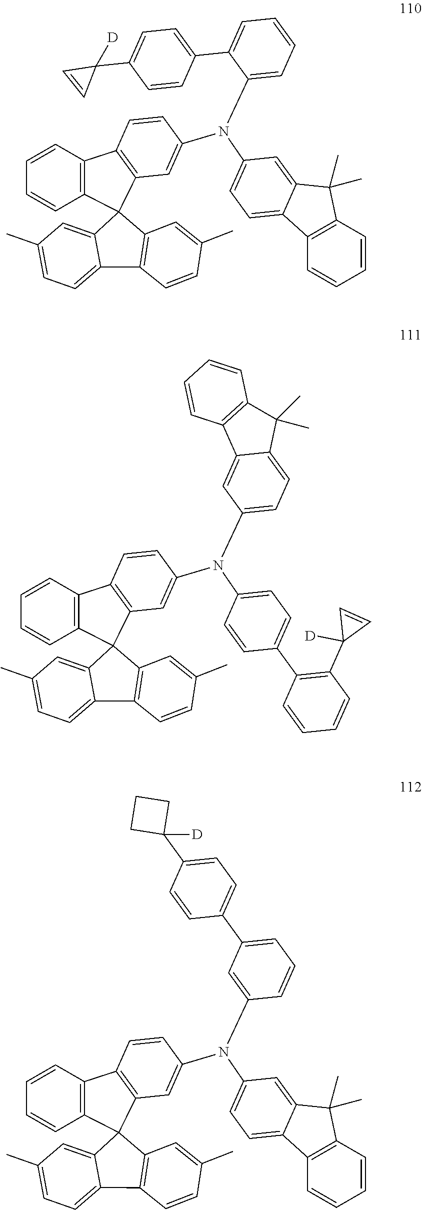 [SMILES: [2H]C1(c2ccc(-c3ccccc3N(c3ccc4c(c3)C(C)(C)c3ccccc3-4)c3ccc4c(c3)C3(c5ccccc5-4)c4cc(C)ccc4-c4ccc(C)cc43)cc2)C=C1 ; [2H]C1(c2ccccc2-c2ccc(N(c3ccc4c(c3)-c3ccccc3C4(C)C)c3ccc4c(c3)C3(c5ccccc5-4)c4cc(C)ccc4-c4ccc(C)cc43)cc2)C=C1 ; [2H]C1(c2ccc(-c3cccc(N(c4ccc5c(c4)C(C)(C)c4ccccc4-5)c4ccc5c(c4)C4(c6ccccc6-5)c5cc(C)ccc5-c5ccc(C)cc54)c3)cc2)CCC1]

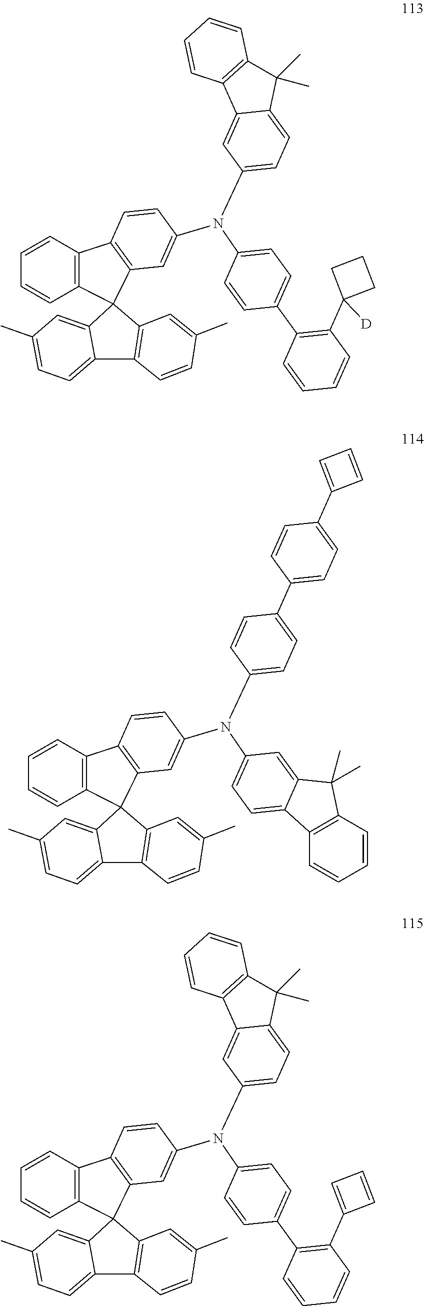 [SMILES: [2H]C1(c2ccccc2-c2ccc(N(c3ccc4c(c3)-c3ccccc3C4(C)C)c3ccc4c(c3)C3(c5ccccc5-4)c4cc(C)ccc4-c4ccc(C)cc43)cc2)CCC1 ; Cc1ccc2c(c1)C1(c3ccccc3-c3ccc(N(c4ccc(-c5ccc(C6=CC=C6)cc5)cc4)c4ccc5c(c4)C(C)(C)c4ccccc4-5)cc31)c1cc(C)ccc1-2 ; Cc1ccc2c(c1)C1(c3ccccc3-c3ccc(N(c4ccc(-c5ccccc5C5=CC=C5)cc4)c4ccc5c(c4)-c4ccccc4C5(C)C)cc31)c1cc(C)ccc1-2]

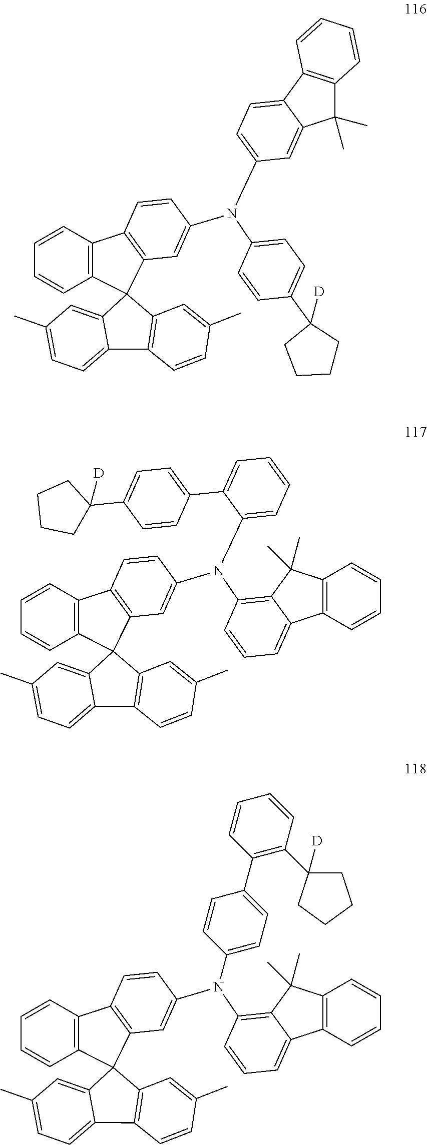

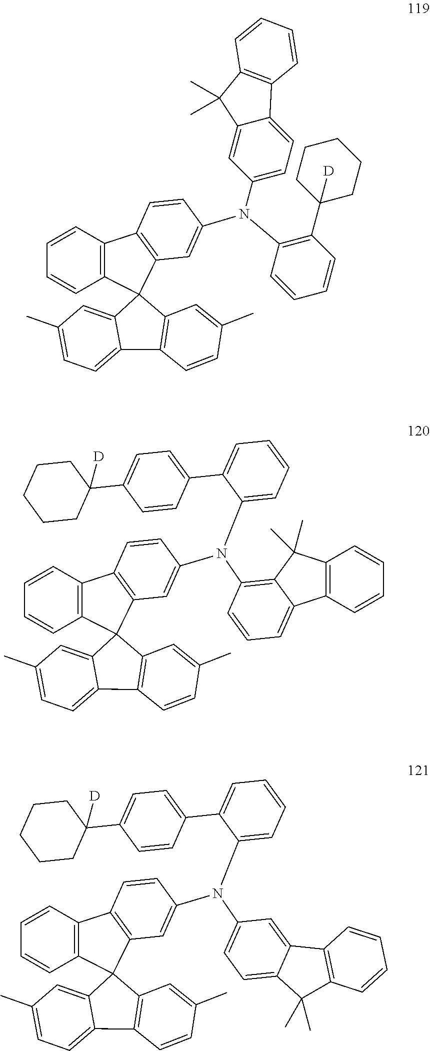

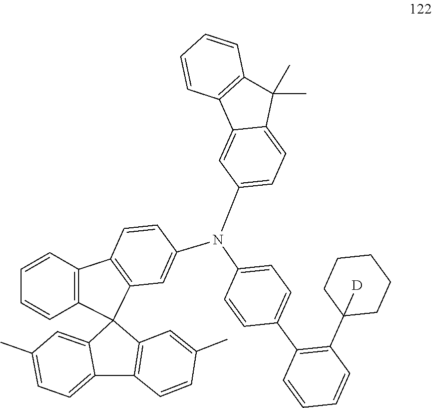

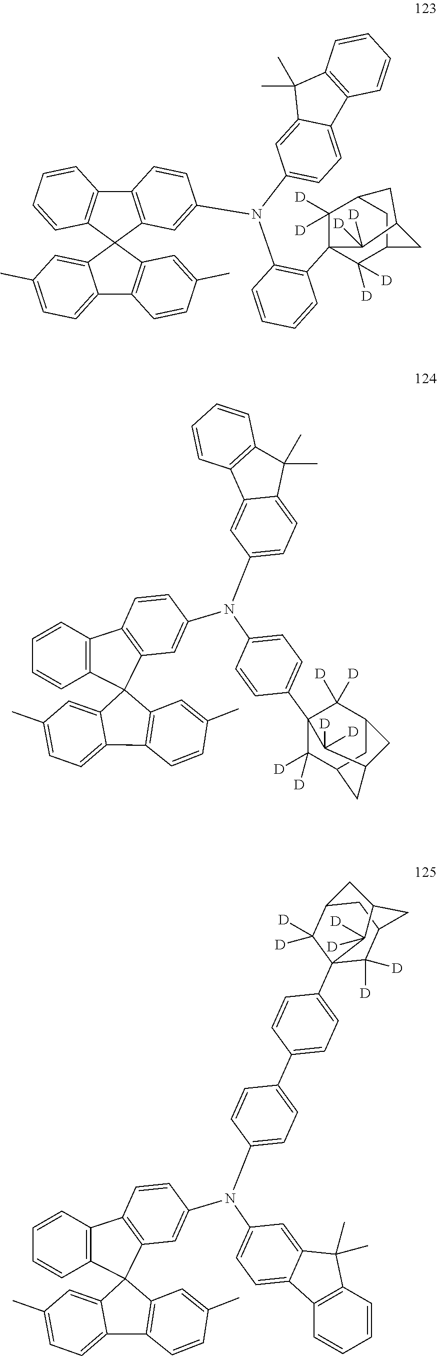 [SMILES: [2H]C1([2H])C2CC3CC(C2)C([2H])([2H])C1(c1ccccc1N(c1ccc2c(c1)C(C)(C)c1ccccc1-2)c1ccc2c(c1)C1(c4ccccc4-2)c2cc(C)ccc2-c2ccc(C)cc21)C3([2H])[2H] ; [2H]C1([2H])C2CC3CC(C2)C([2H])([2H])C1(c1ccc(N(c2ccc4c(c2)-c2ccccc2C4(C)C)c2ccc4c(c2)C2(c5ccccc5-4)c4cc(C)ccc4-c4ccc(C)cc42)cc1)C3([2H])[2H] ; [2H]C1([2H])C2CC3CC(C2)C([2H])([2H])C1(c1ccc(-c2ccc(N(c4ccc5c(c4)C(C)(C)c4ccccc4-5)c4ccc5c(c4)C4(c6ccccc6-5)c5cc(C)ccc5-c5ccc(C)cc54)cc2)cc1)C3([2H])[2H]]

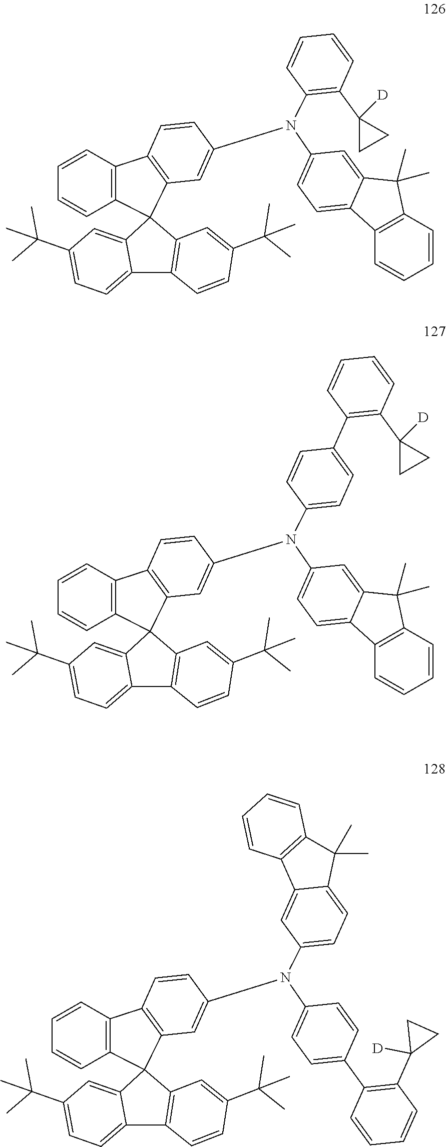 [SMILES: [2H]C1(c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)CC1 ; [2H]C1(c2ccccc2-c2ccc(N(c3ccc4c(c3)C(C)(C)c3ccccc3-4)c3ccc4c(c3)C3(c5ccccc5-4)c4cc(C(C)(C)C)ccc4-c4ccc(C(C)(C)C)cc43)cc2)CC1 ; [2H]C1(c2ccccc2-c2ccc(N(c3ccc4c(c3)-c3ccccc3C4(C)C)c3ccc4c(c3)C3(c5ccccc5-4)c4cc(C(C)(C)C)ccc4-c4ccc(C(C)(C)C)cc43)cc2)CC1]

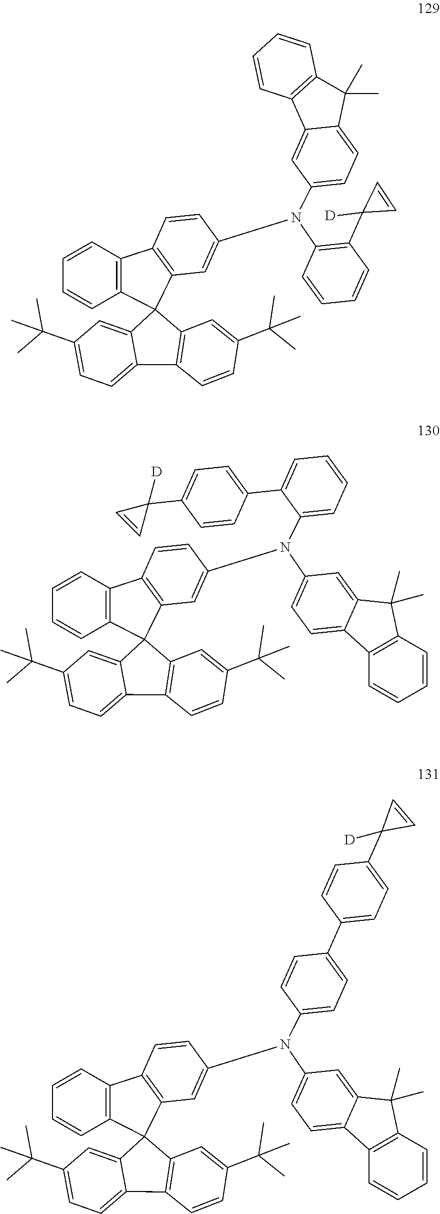 [SMILES: [2H]C1(c2ccccc2N(c2ccc3c(c2)-c2ccccc2C3(C)C)c2ccc3c(c2)C2(c4ccccc4-3)c3cc(C(C)(C)C)ccc3-c3ccc(C(C)(C)C)cc32)C=C1 ; [2H]C1(c2ccc(-c3ccccc3N(c3ccc4c(c3)C(C)(C)c3ccccc3-4)c3ccc4c(c3)C3(c5ccccc5-4)c4cc(C(C)(C)C)ccc4-c4ccc(C(C)(C)C)cc43)cc2)C=C1 ; [2H]C1(c2ccc(-c3ccc(N(c4ccc5c(c4)C(C)(C)c4ccccc4-5)c4ccc5c(c4)C4(c6ccccc6-5)c5cc(C(C)(C)C)ccc5-c5ccc(C(C)(C)C)cc54)cc3)cc2)C=C1]

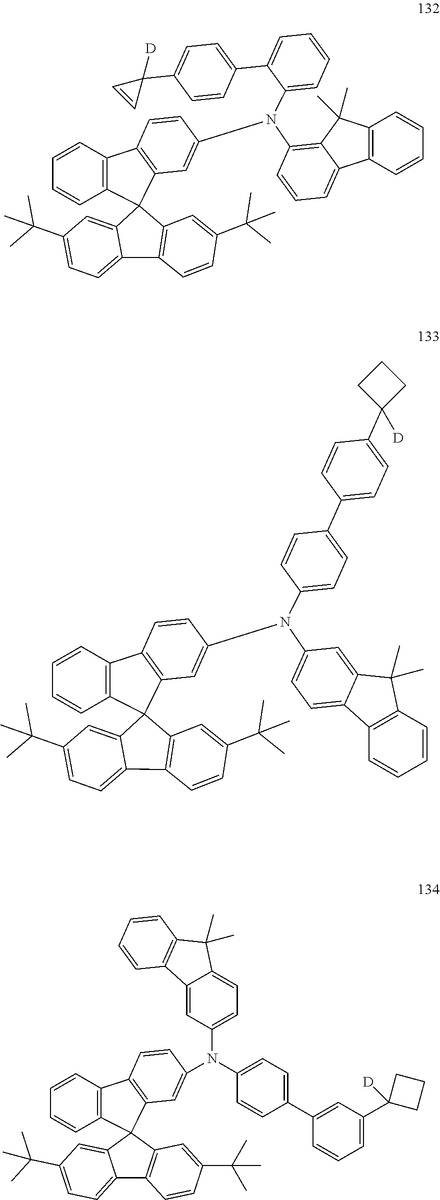 [SMILES: [2H]C1(c2ccc(-c3ccccc3N(c3ccc4c(c3)C3(c5ccccc5-4)c4cc(C(C)(C)C)ccc4-c4ccc(C(C)(C)C)cc43)c3cccc4c3C(C)(C)c3ccccc3-4)cc2)C=C1 ; [2H]C1(c2ccc(-c3ccc(N(c4ccc5c(c4)C(C)(C)c4ccccc4-5)c4ccc5c(c4)C4(c6ccccc6-5)c5cc(C(C)(C)C)ccc5-c5ccc(C(C)(C)C)cc54)cc3)cc2)CCC1 ; [2H]C1(c2cccc(-c3ccc(N(c4ccc5c(c4)-c4ccccc4C5(C)C)c4ccc5c(c4)C4(c6ccccc6-5)c5cc(C(C)(C)C)ccc5-c5ccc(C(C)(C)C)cc54)cc3)c2)CCC1]

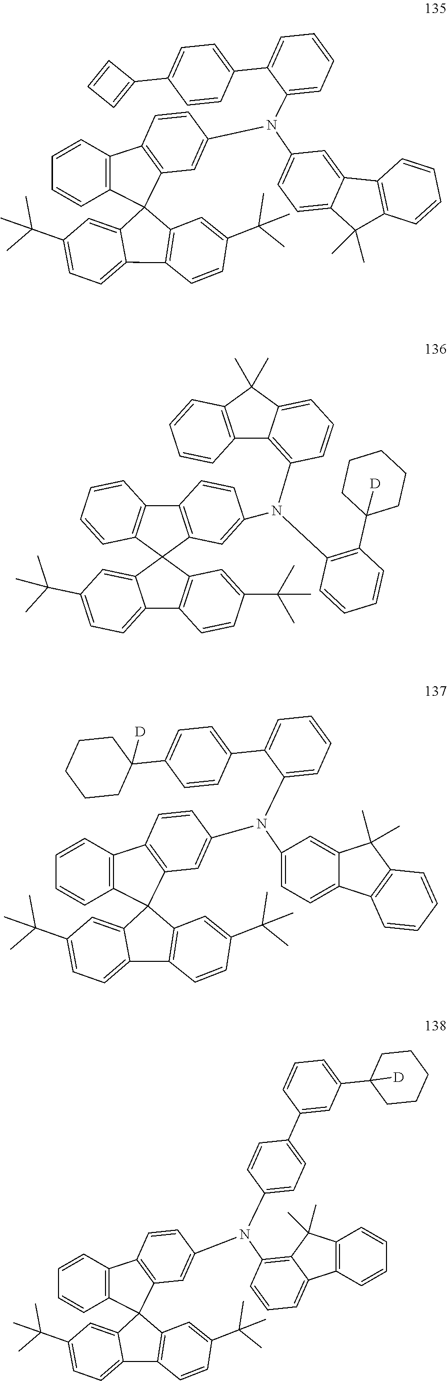

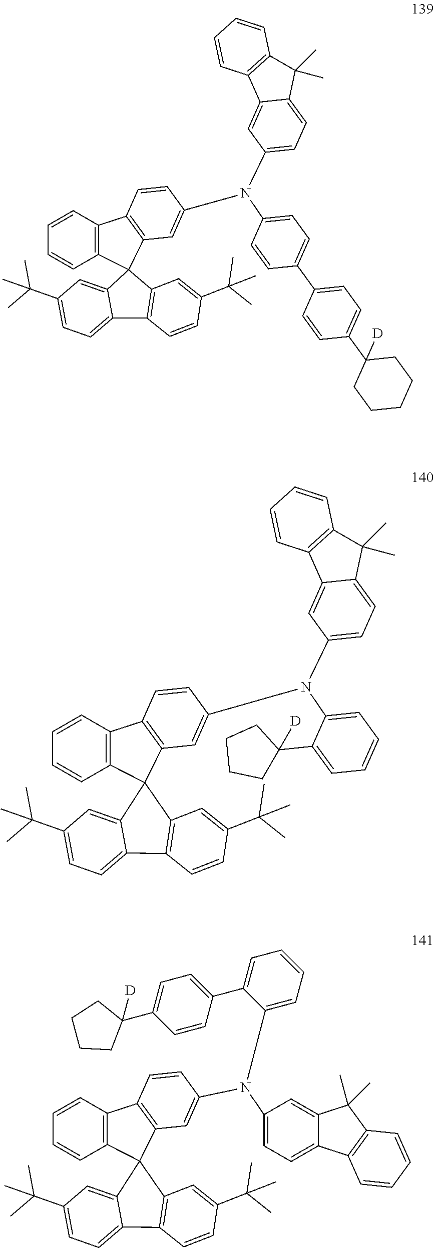

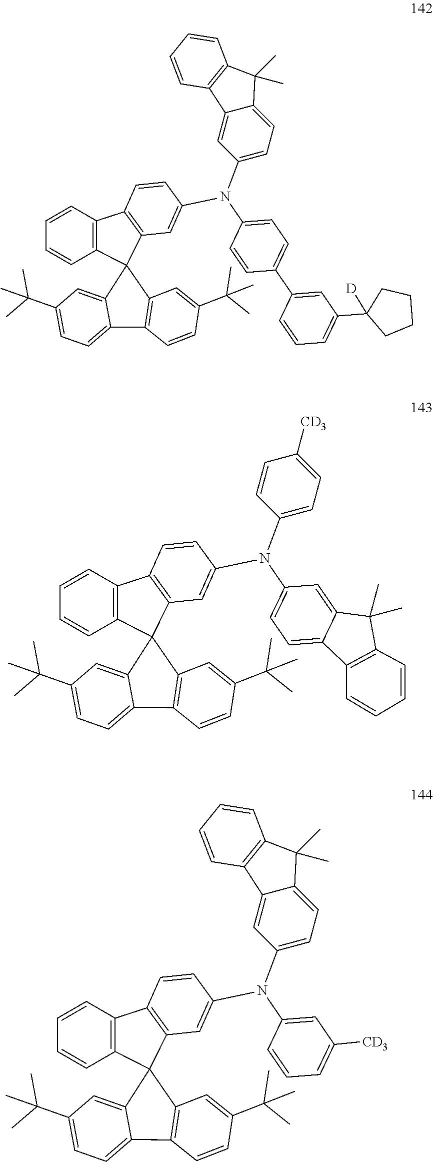 [SMILES: [2H]C1(c2cccc(-c3ccc(N(c4ccc5c(c4)-c4ccccc4C5(C)C)c4ccc5c(c4)C4(c6ccccc6-5)c5cc(C(C)(C)C)ccc5-c5ccc(C(C)(C)C)cc54)cc3)c2)CCCC1 ; [2H]C([2H])([2H])c1ccc(N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1 ; [2H]C([2H])([2H])c1cccc(N(c2ccc3c(c2)-c2ccccc2C3(C)C)c2ccc3c(c2)C2(c4ccccc4-3)c3cc(C(C)(C)C)ccc3-c3ccc(C(C)(C)C)cc32)c1]

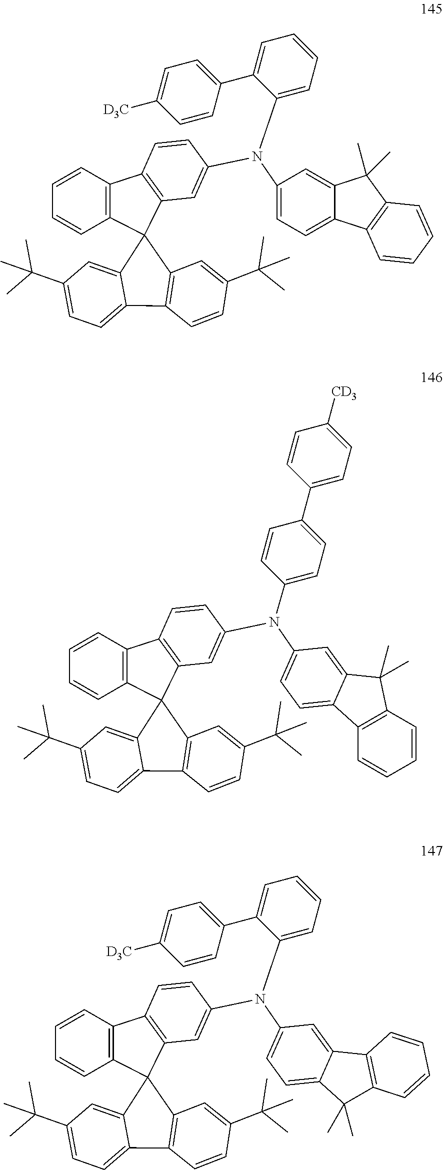 [SMILES: [2H]C([2H])([2H])c1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1 ; [2H]C([2H])([2H])c1ccc(-c2ccc(N(c3ccc4c(c3)C(C)(C)c3ccccc3-4)c3ccc4c(c3)C3(c5ccccc5-4)c4cc(C(C)(C)C)ccc4-c4ccc(C(C)(C)C)cc43)cc2)cc1 ; [2H]C([2H])([2H])c1ccc(-c2ccccc2N(c2ccc3c(c2)-c2ccccc2C3(C)C)c2ccc3c(c2)C2(c4ccccc4-3)c3cc(C(C)(C)C)ccc3-c3ccc(C(C)(C)C)cc32)cc1]

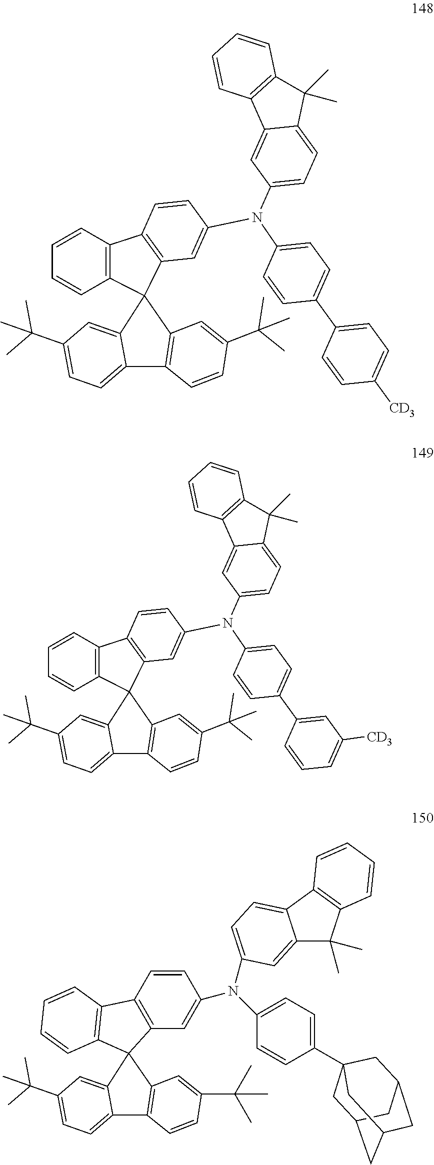 [SMILES: [2H]C([2H])([2H])c1ccc(-c2ccc(N(c3ccc4c(c3)-c3ccccc3C4(C)C)c3ccc4c(c3)C3(c5ccccc5-4)c4cc(C(C)(C)C)ccc4-c4ccc(C(C)(C)C)cc43)cc2)cc1 ; [2H]C([2H])([2H])c1cccc(-c2ccc(N(c3ccc4c(c3)-c3ccccc3C4(C)C)c3ccc4c(c3)C3(c5ccccc5-4)c4cc(C(C)(C)C)ccc4-c4ccc(C(C)(C)C)cc43)cc2)c1 ; CC(C)(C)c1ccc2c(c1)C1(c3ccccc3-c3ccc(N(c4ccc(C56CC7CC(CC(C7)C5)C6)cc4)c4ccc5c(c4)C(C)(C)c4ccccc4-5)cc31)c1cc(C(C)(C)C)ccc1-2]

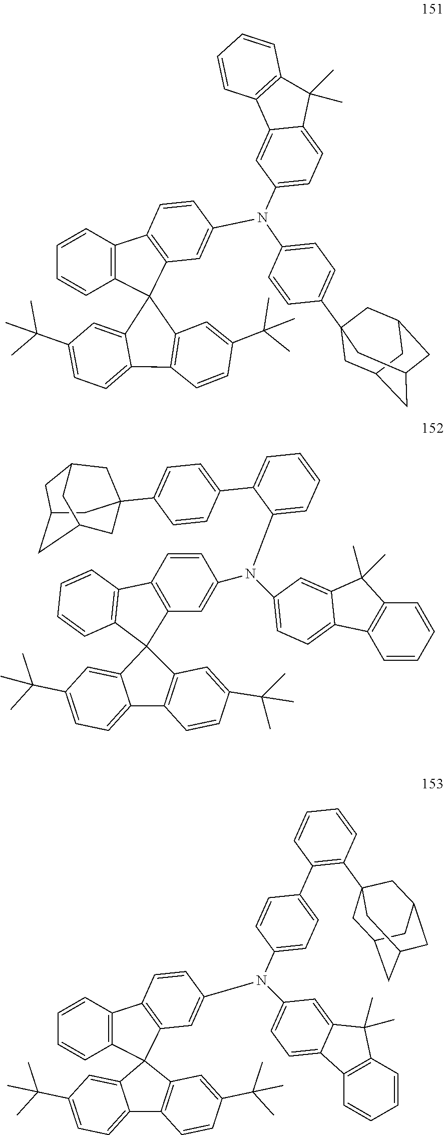 [SMILES: CC(C)(C)c1ccc2c(c1)C1(c3ccccc3-c3ccc(N(c4ccc(C56CC7CC(CC(C7)C5)C6)cc4)c4ccc5c(c4)-c4ccccc4C5(C)C)cc31)c1cc(C(C)(C)C)ccc1-2 ; CC(C)(C)c1ccc2c(c1)C1(c3ccccc3-c3ccc(N(c4ccc5c(c4)C(C)(C)c4ccccc4-5)c4ccccc4-c4ccc(C56CC7CC(CC(C7)C5)C6)cc4)cc31)c1cc(C(C)(C)C)ccc1-2 ; CC(C)(C)c1ccc2c(c1)C1(c3ccccc3-c3ccc(N(c4ccc(-c5ccccc5C56CC7CC(CC(C7)C5)C6)cc4)c4ccc5c(c4)C(C)(C)c4ccccc4-5)cc31)c1cc(C(C)(C)C)ccc1-2]

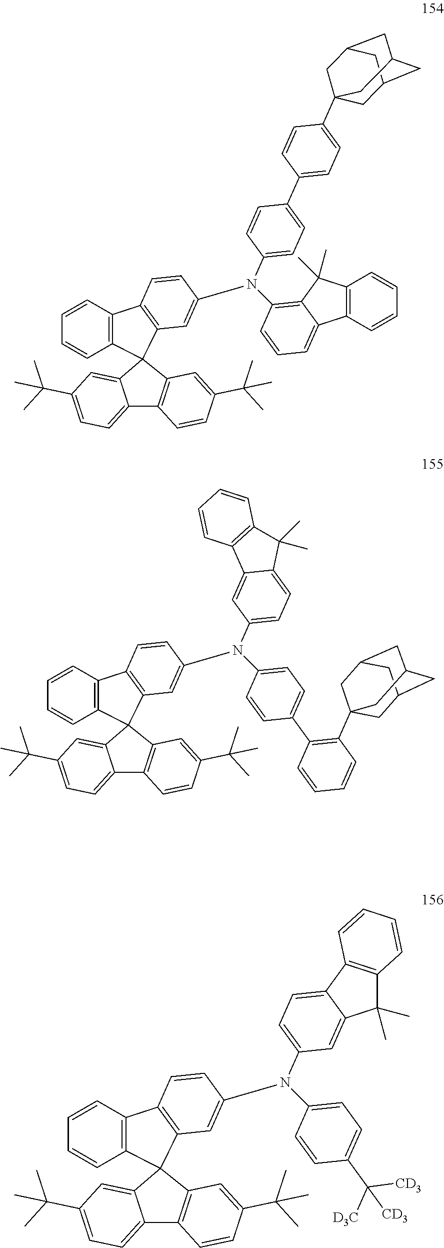 [SMILES: CC(C)(C)c1ccc2c(c1)C1(c3ccccc3-c3ccc(N(c4ccc(-c5ccc(C67CC8CC(CC(C8)C6)C7)cc5)cc4)c4cccc5c4C(C)(C)c4ccccc4-5)cc31)c1cc(C(C)(C)C)ccc1-2 ; CC(C)(C)c1ccc2c(c1)C1(c3ccccc3-c3ccc(N(c4ccc(-c5ccccc5C56CC7CC(CC(C7)C5)C6)cc4)c4ccc5c(c4)-c4ccccc4C5(C)C)cc31)c1cc(C(C)(C)C)ccc1-2 ; [2H]C([2H])([2H])C(c1ccc(N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1)(C([2H])([2H])[2H])C([2H])([2H])[2H]]

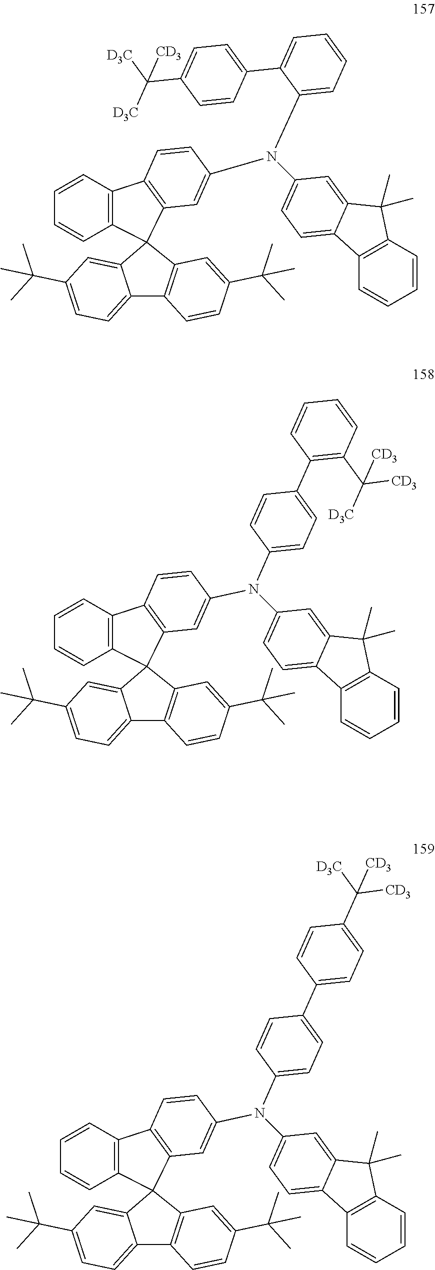

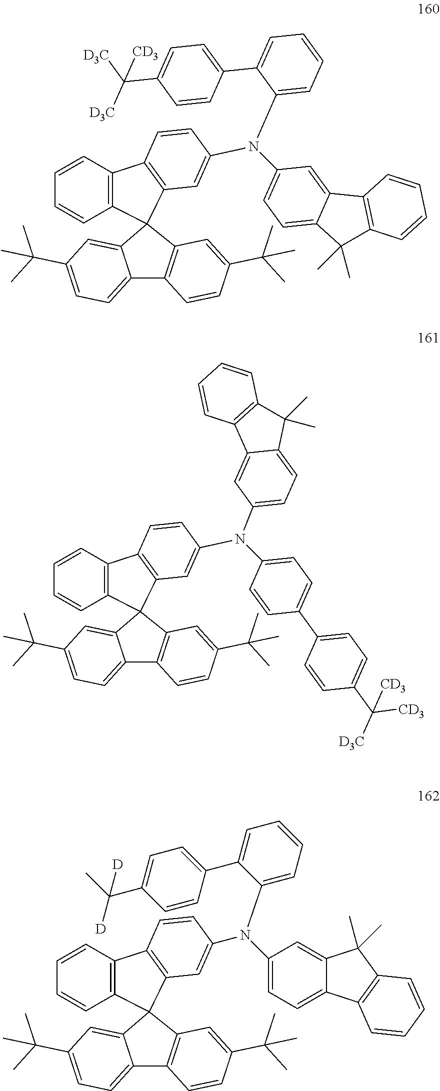 [SMILES: [2H]C([2H])([2H])C(c1ccc(-c2ccccc2N(c2ccc3c(c2)-c2ccccc2C3(C)C)c2ccc3c(c2)C2(c4ccccc4-3)c3cc(C(C)(C)C)ccc3-c3ccc(C(C)(C)C)cc32)cc1)(C([2H])([2H])[2H])C([2H])([2H])[2H] ; [2H]C([2H])([2H])C(c1ccc(-c2ccc(N(c3ccc4c(c3)-c3ccccc3C4(C)C)c3ccc4c(c3)C3(c5ccccc5-4)c4cc(C(C)(C)C)ccc4-c4ccc(C(C)(C)C)cc43)cc2)cc1)(C([2H])([2H])[2H])C([2H])([2H])[2H] ; [2H]C([2H])(C)c1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1]

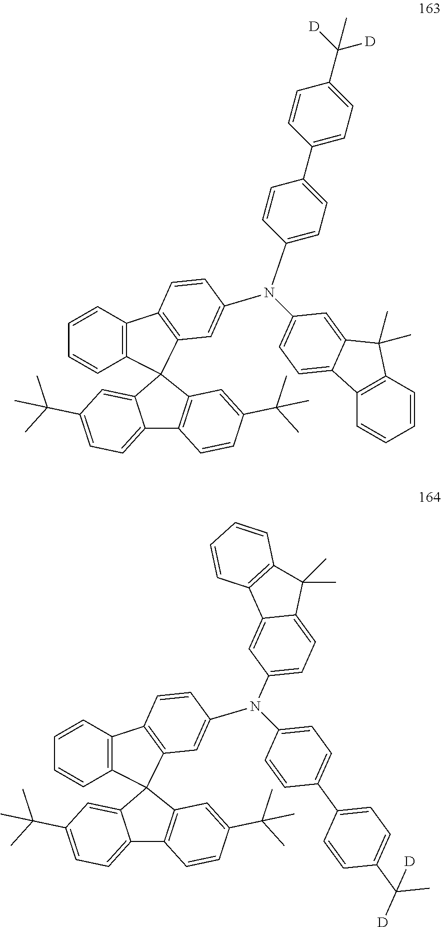 [SMILES: [2H]C([2H])(C)c1ccc(-c2ccc(N(c3ccc4c(c3)C(C)(C)c3ccccc3-4)c3ccc4c(c3)C3(c5ccccc5-4)c4cc(C(C)(C)C)ccc4-c4ccc(C(C)(C)C)cc43)cc2)cc1 ; [2H]C([2H])(C)c1ccc(-c2ccc(N(c3ccc4c(c3)-c3ccccc3C4(C)C)c3ccc4c(c3)C3(c5ccccc5-4)c4cc(C(C)(C)C)ccc4-c4ccc(C(C)(C)C)cc43)cc2)cc1]

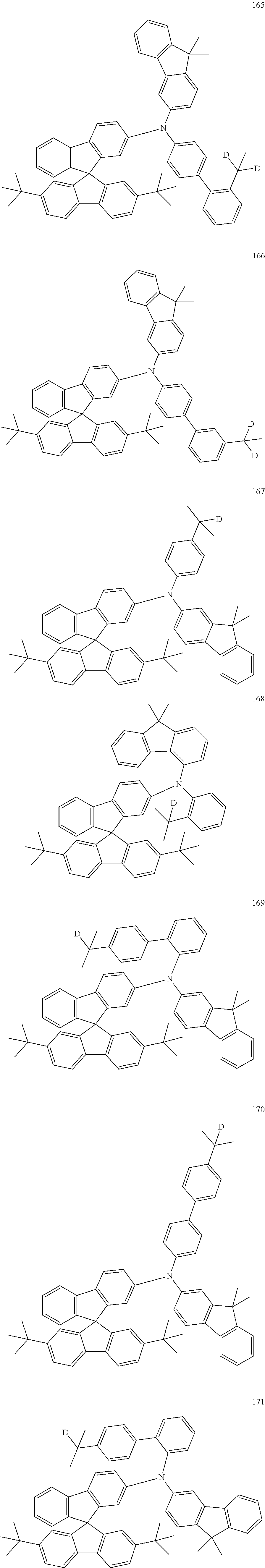

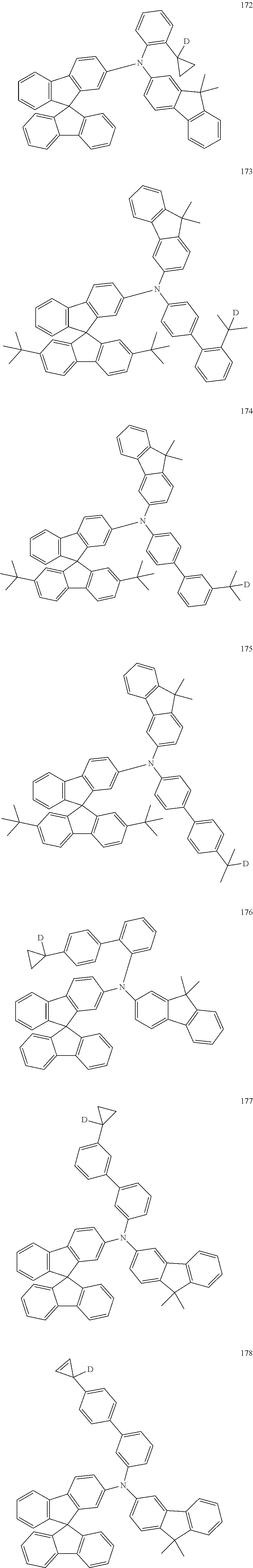

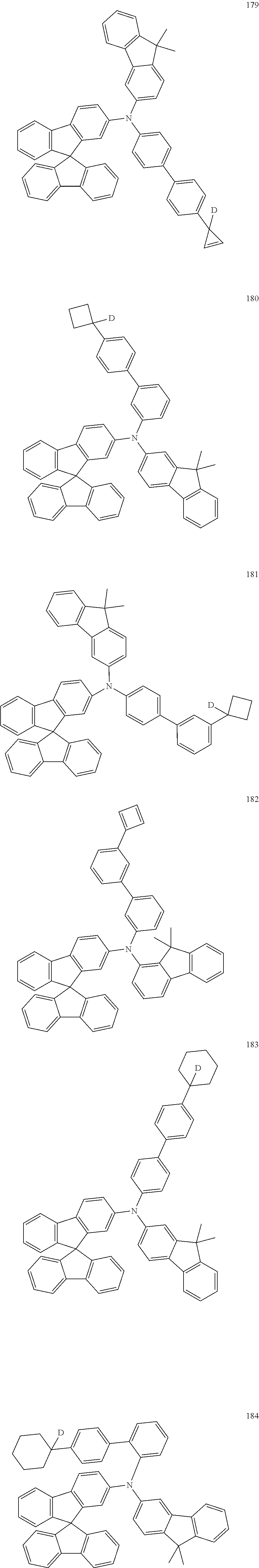

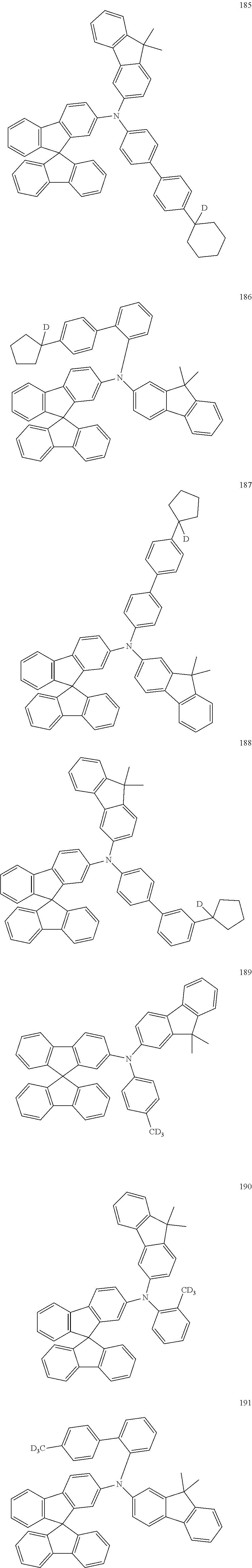

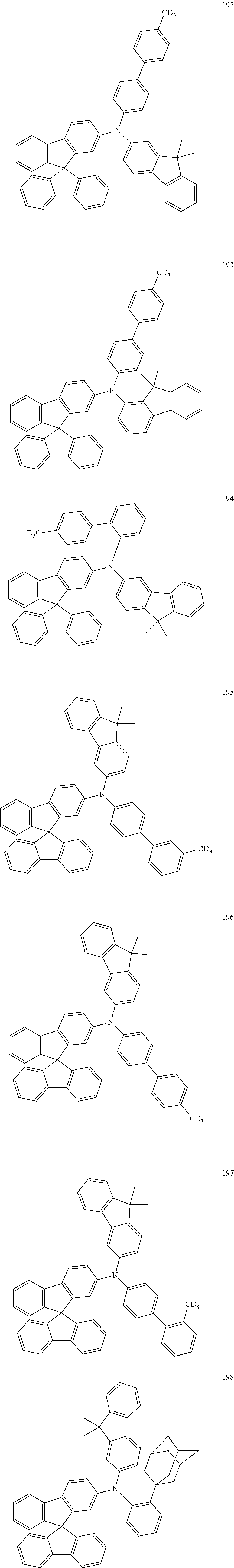

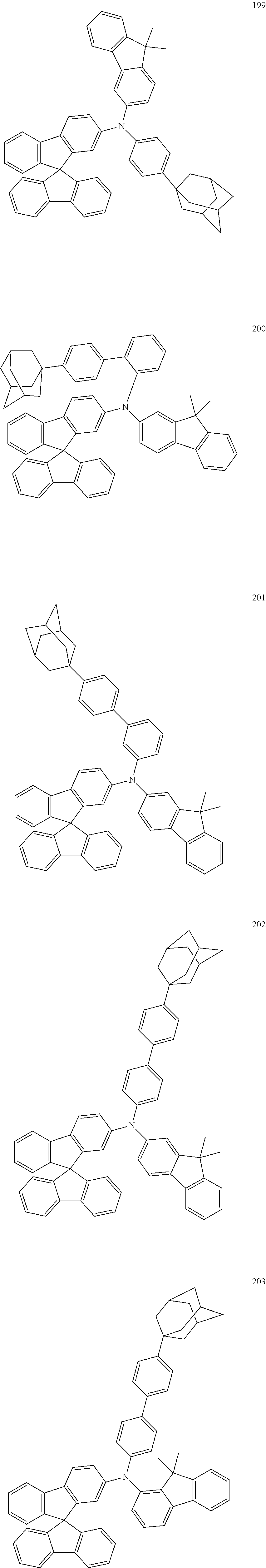

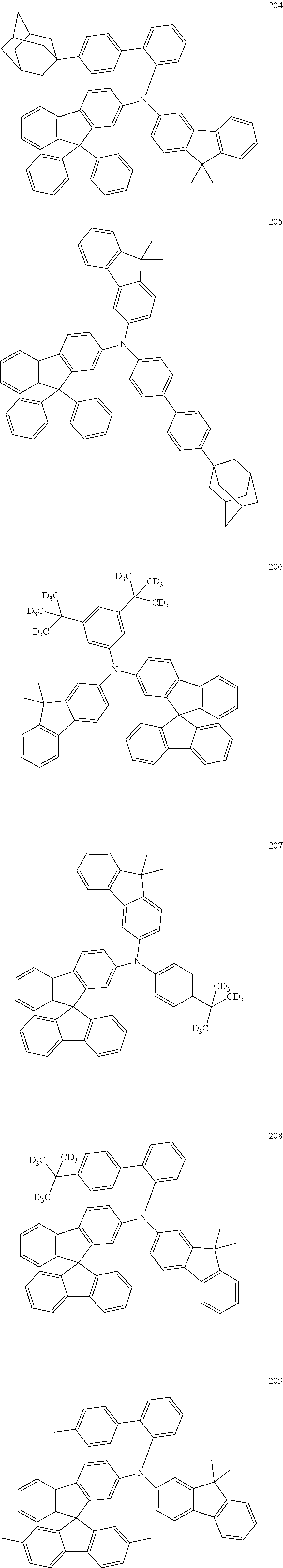

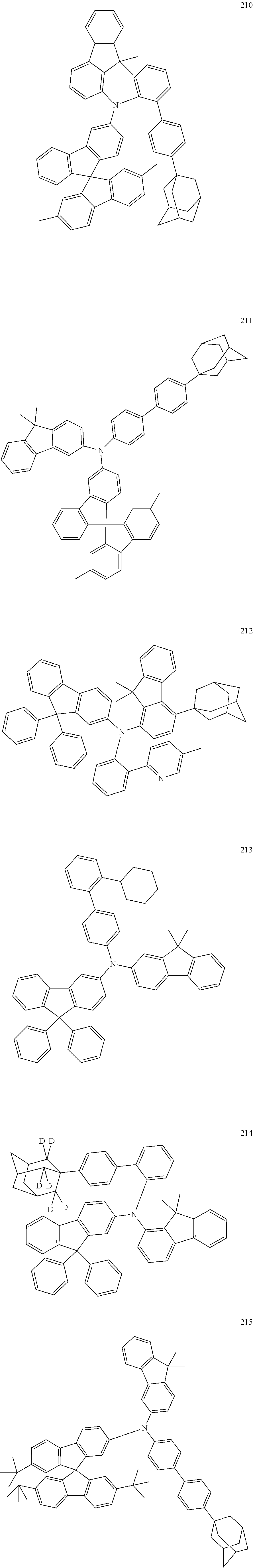

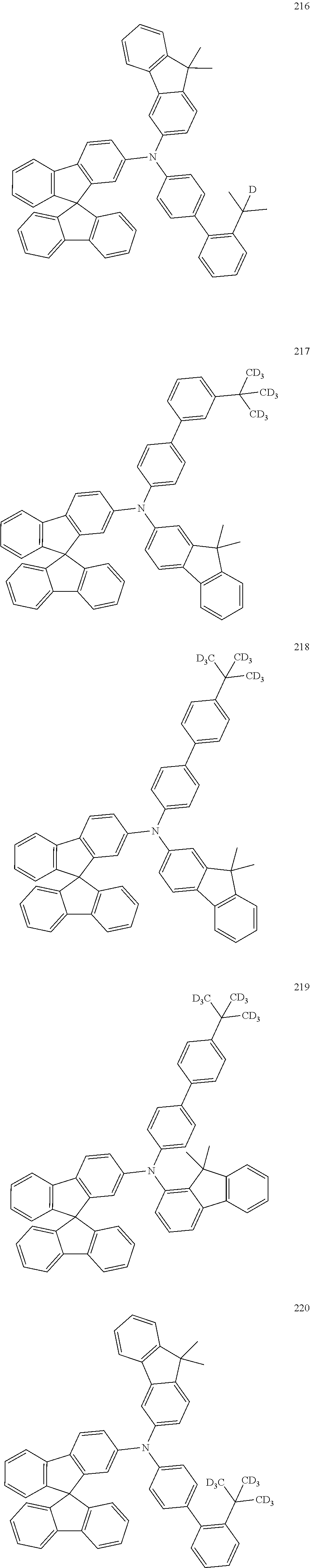

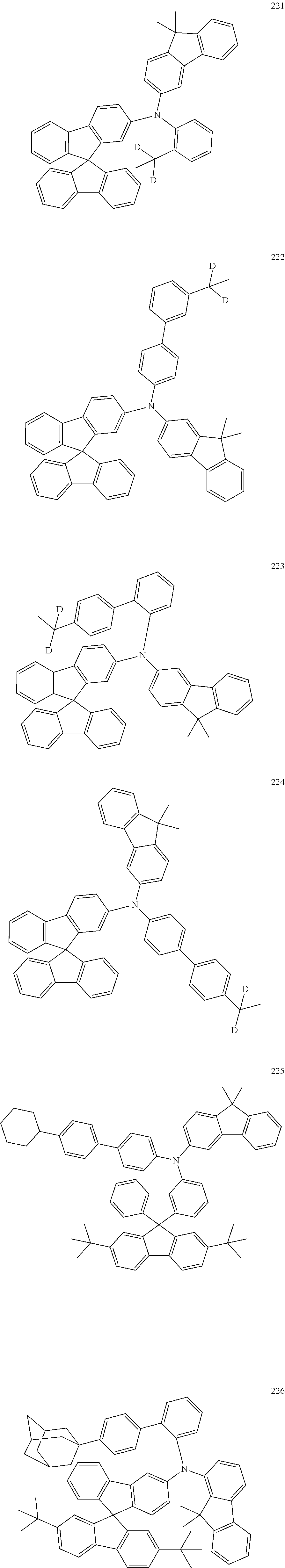

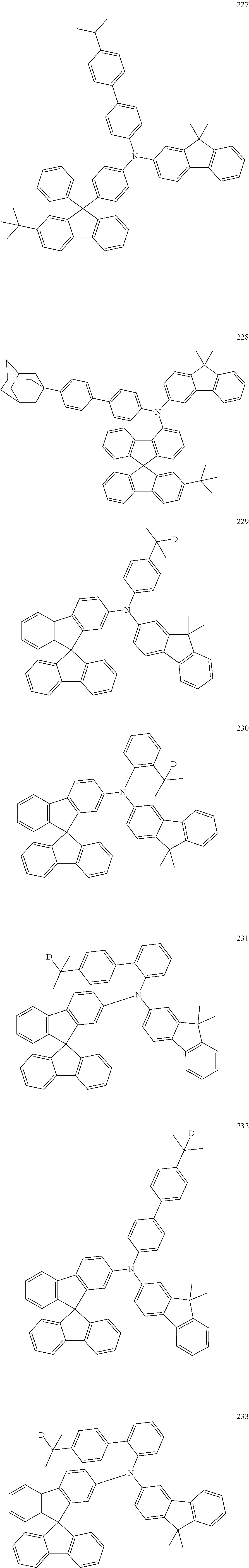

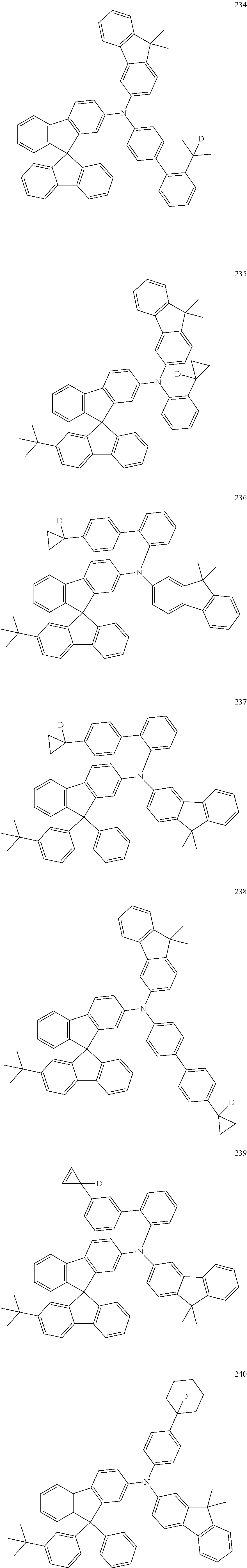

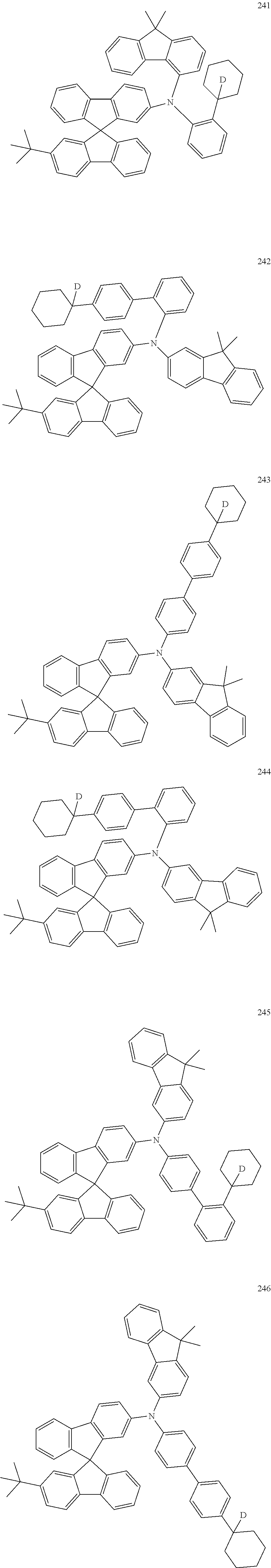

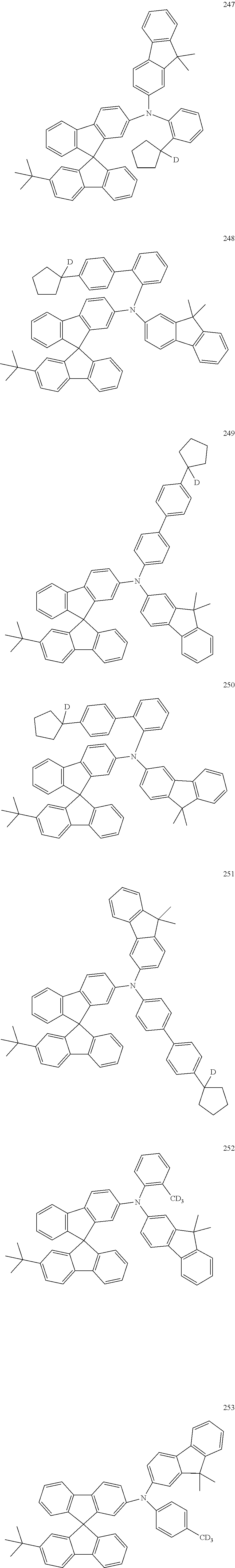

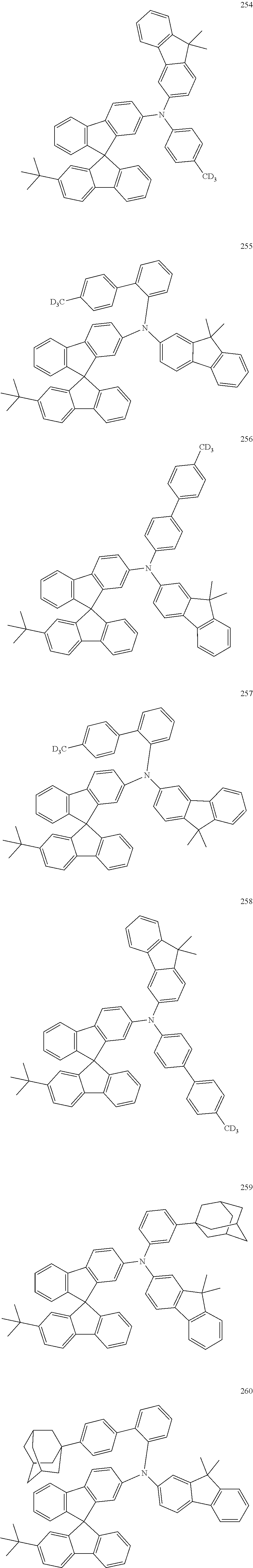

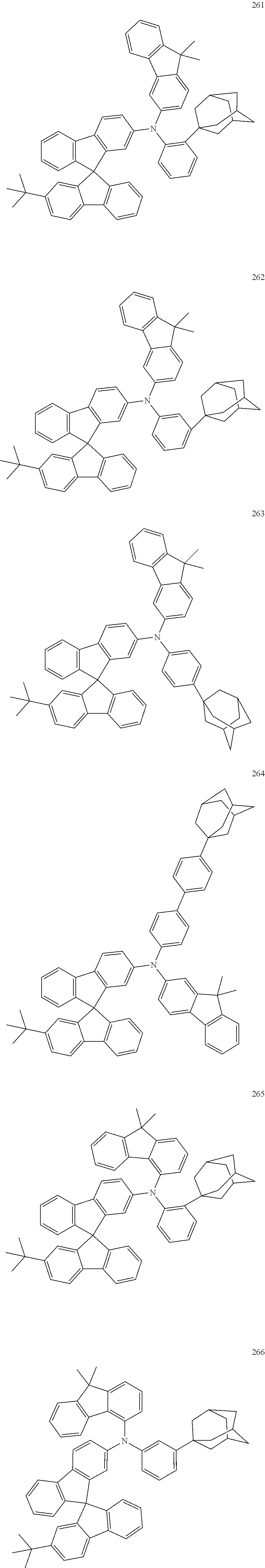

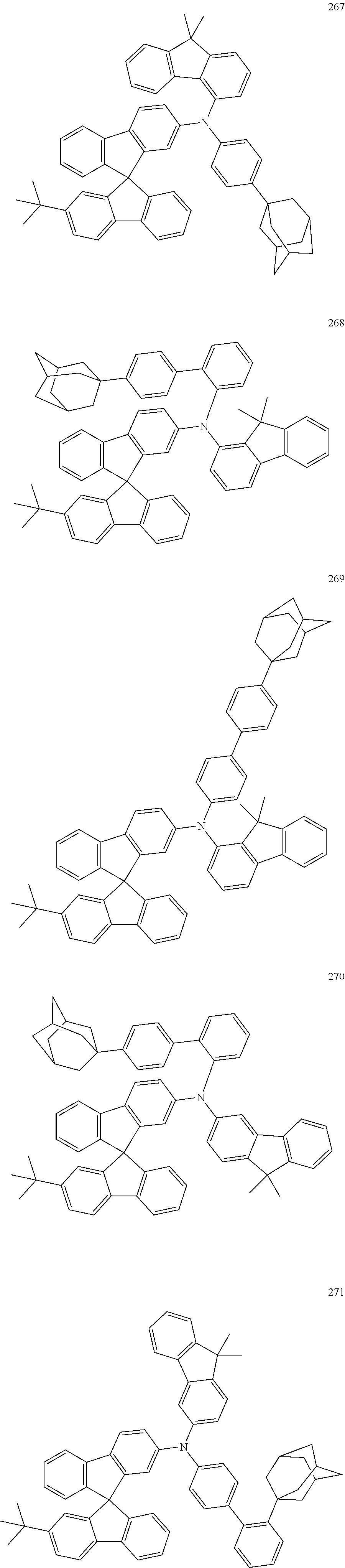

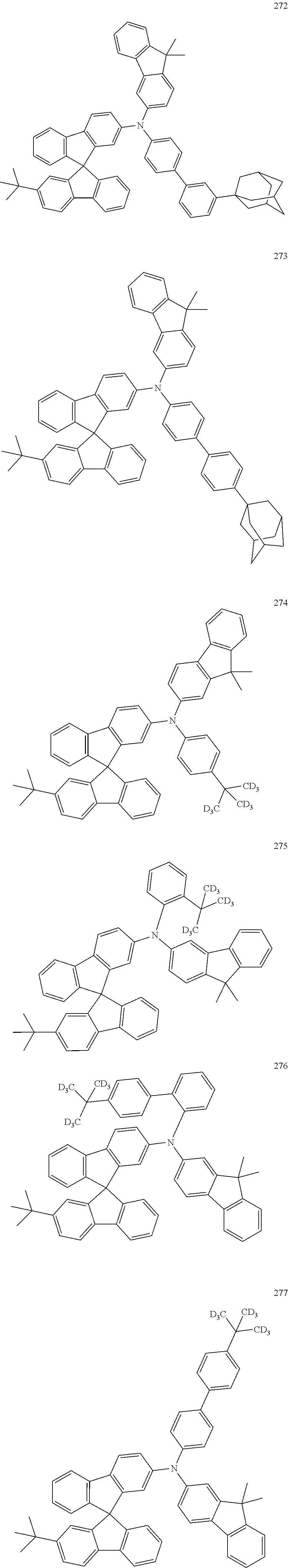

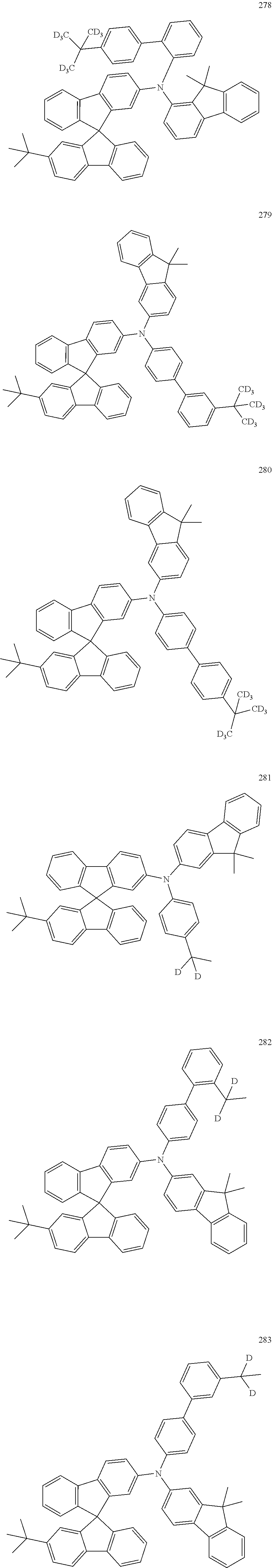

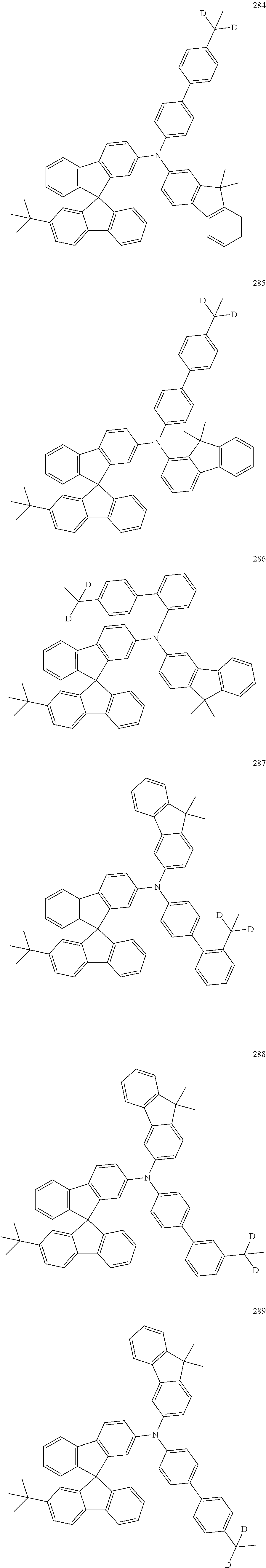

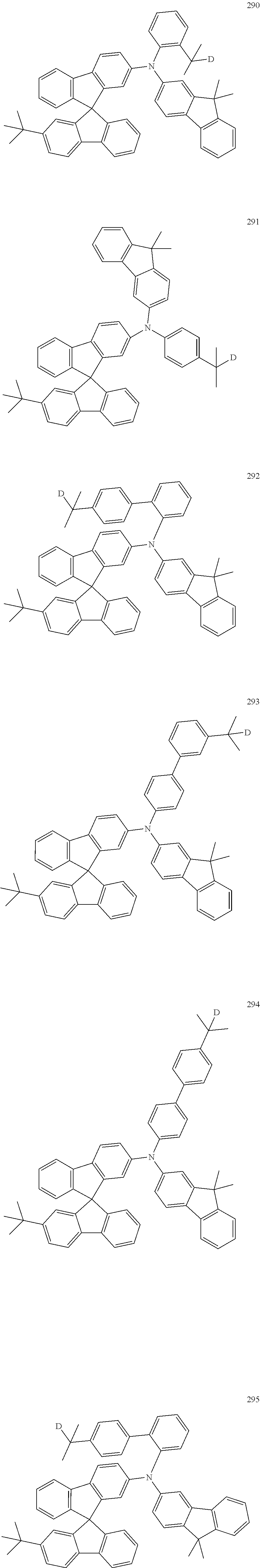

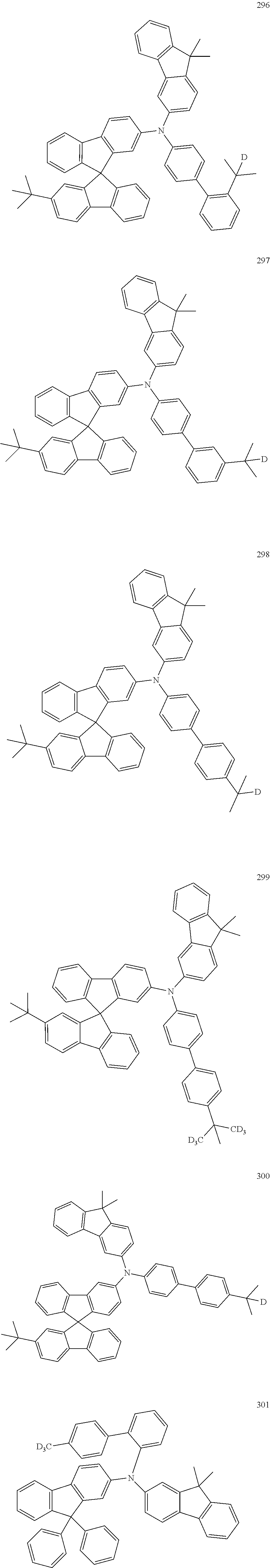

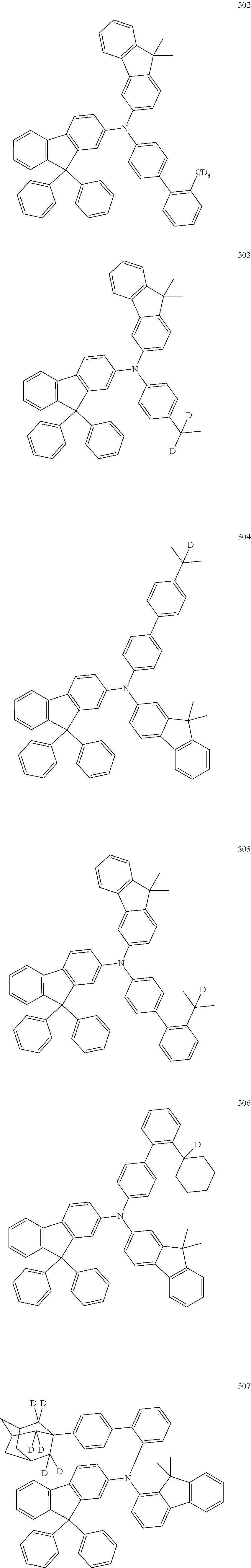

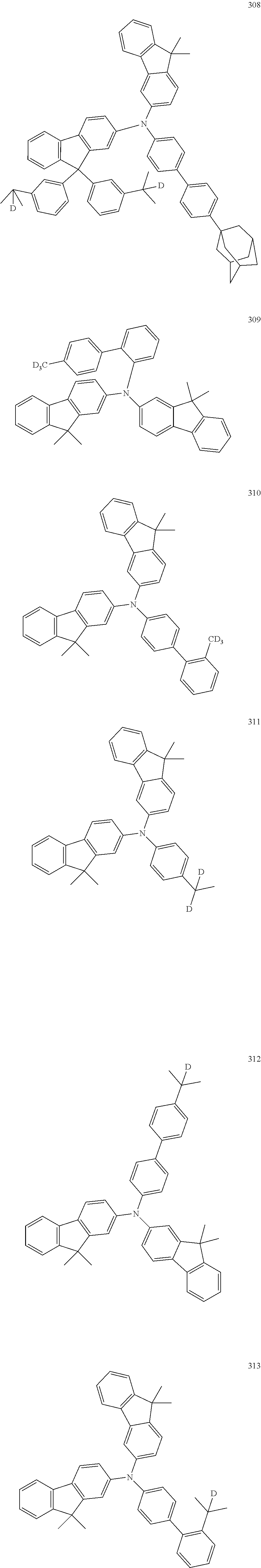

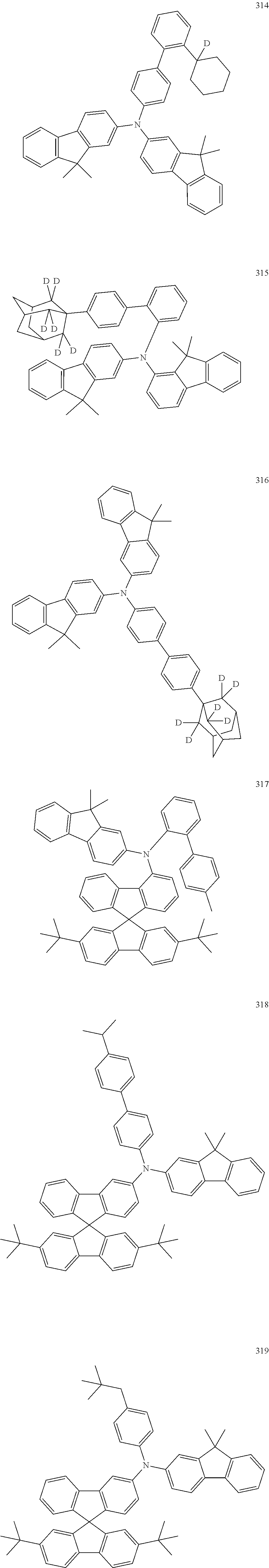

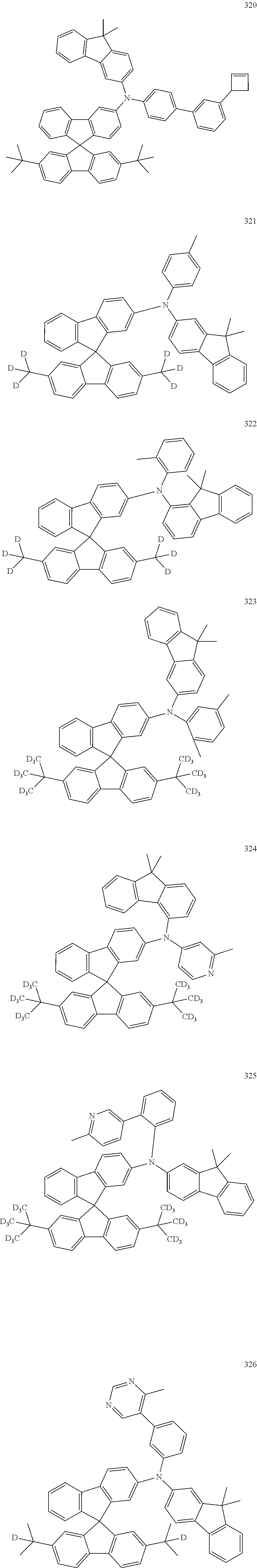

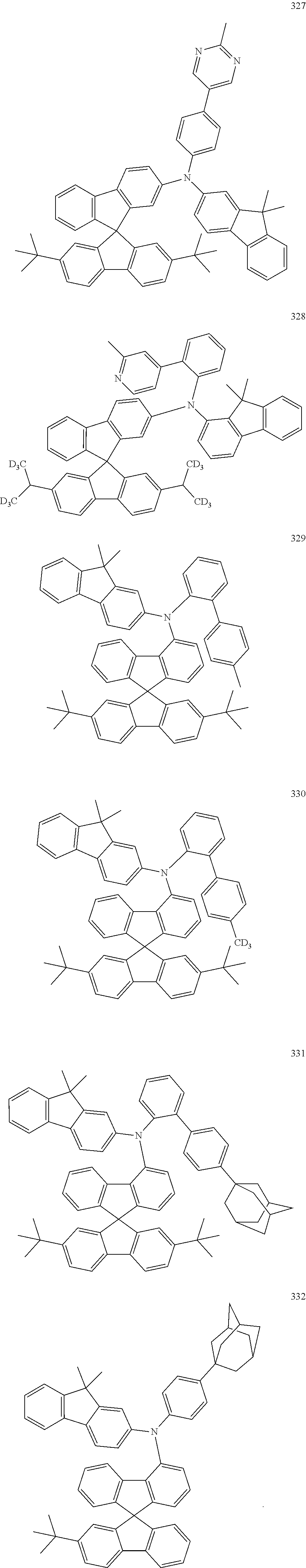

An organic electroluminescent device, including: an anode, a hole injection layer, a hole transport layer, a light-emitting layer, an electron transport layer, an electron injection layer and a cathode, wherein any one of the hole injection layer, the hole transport layer, the light-emitting layer, the electron transport layer and the electron injection layer contains at least one of the novel organic electroluminescent compounds above.

Further, the hole transport layer contains at least one of the novel organic electroluminescent compounds above.

An electronic display device, containing the organic electroluminescent device above.

The room temperature in the present disclosure is 25±5°.

The present disclosure has following beneficial effects:

The main structure of the organic electroluminescent compound designed in the present disclosure is a fluorene-based compound, the main structure has rich electron cloud density, good carrier migration rate and thermal stability, and the organic electroluminescent compound designed with this structure as main body has good stability and hole mobility. Particularly, when alkyl groups such as straight alkyl group, branched alkyl group, cycloalkyl group or adamantane group and a phenyl group or a biphenyl group substituted by deuterated alkyl thereof are introduced into the branched substituent R₃, such substituents have very strong electron donating property, and can greatly improve the electron cloud density of material molecules, further improving the hole mobility of the material, and further effectively improving the light-emitting efficiency of the device. Moreover, the HOMO energy level and the LUMO energy level of the material are adjusted by adjusting the electron donating capability of the substituent group, so that the material has rich collocation application, and the voltage of the device can be greatly reduced by adjusting the collocation of the device, thereby achieving the purpose of energy conservation. Meanwhile, experiments prove and indicate that the organic electroluminescent compound designed in the present disclosure has better thermal stability, and the OLED devices manufactured using such compound have, by contrast, higher efficiency and lower voltage.

### BRIEF DESCRIPTION OF DRAWINGS

FIG. 1 is a structural schematic diagram of an organic electroluminescent device in the present disclosure.

The reference signs in the drawing are illustrated as follows:

1—anode, 2—hole injection layer, 3—hole transport layer, 4—light-emitting layer, 5—electron transport layer, 6—electron injection layer, and 7—cathode.

FIG. 2 is a graph showing a thermogravimetric temperature curve of a novel organic electroluminescent compound 5, and it can be seen from FIG. 2 that a thermogravimetric temperature Td of the novel organic electroluminescent compound 5 is 417.58° C.

FIG. 3 shows curves of light-emitting service life of organic electroluminescent devices prepared in Application Example 1 and comparative example, and it can be seen from FIG. 3 that the light-emitting service life T97% of the organic electroluminescent devices prepared in Application Example 1 and comparative example are 274 h and 251 h, respectively.

FIG. 4 shows curves of light-emitting efficiency of the organic electroluminescent devices prepared in Application Example 1 and comparative example, and it can be seen from FIG. 4 that the light-emitting efficiency of the organic electroluminescent devices prepared in Application Example 1 and Comparative Example 1 are 12.4 and 10.2, respectively.

FIG. 5 shows voltage-brightness curves of the organic electroluminescent devices prepared in Application Example 1 and comparative example, and it can be seen from FIG. 5 that starting voltages of the organic electroluminescent devices prepared in Application Example 1 and Comparative Example 1 are 4.02 V and 4.54 V, respectively.

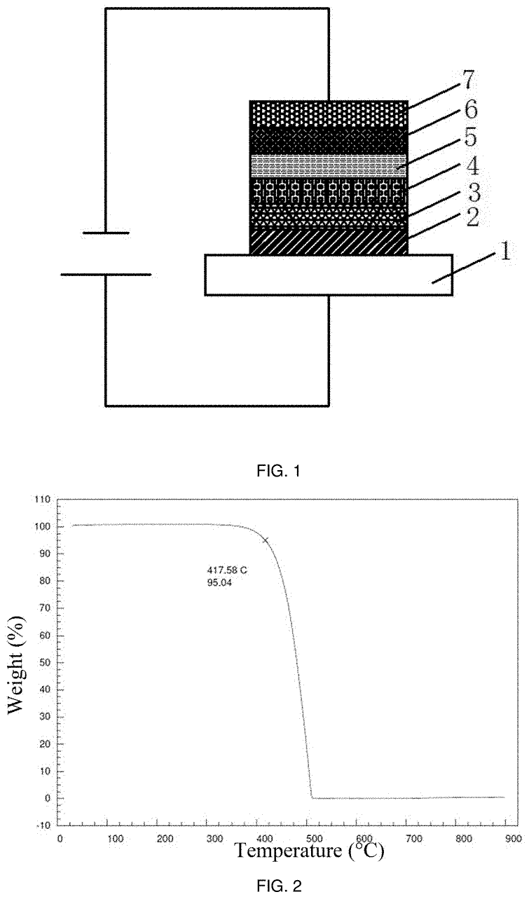

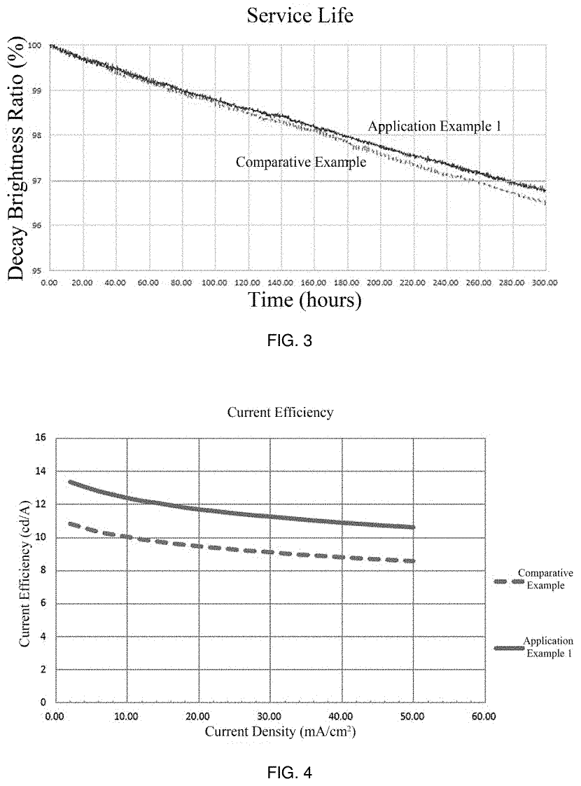

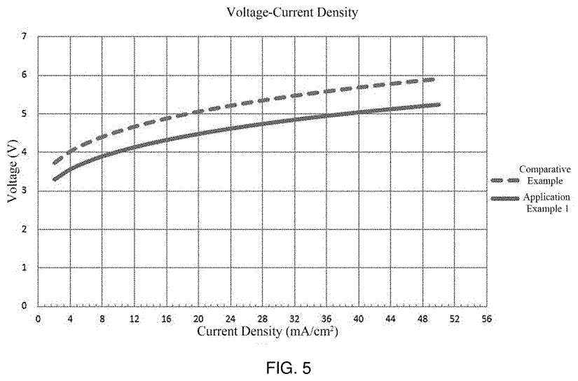

### DETAILED DESCRIPTION OF EMBODIMENTS

If no specific conditions are specified in the examples, they are carried out under normal conditions or conditions recommended by manufacturers. If manufacturers of reagents or apparatuses used are not specified, they are conventional products commercially available.

### Example 1

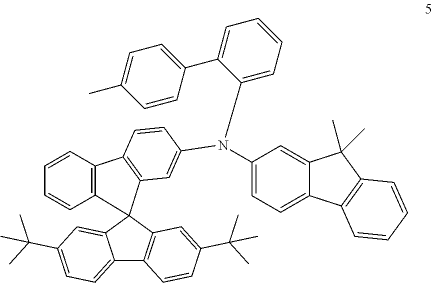 [SMILES: Cc1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1]

A synthetic method of a novel organic electroluminescent compound 5 is as follows:

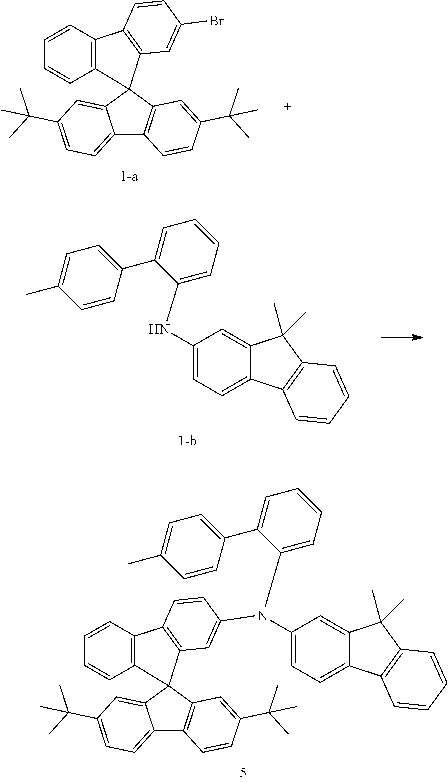 [SMILES: CC(C)(C)c1ccc2c(c1)C1(c3ccccc3-c3ccc(Br)cc31)c1cc(C(C)(C)C)ccc1-2 ; Cc1ccc(-c2ccccc2Nc2ccc3c(c2)C(C)(C)c2ccccc2-3)cc1 ; Cc1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1]

Under the protection of nitrogen, compound 1-a (4 g, 507.50 g/mol, 7.88 mmol), compound 1-b (1 eq, 2.96 g, 375.50 g/mol, 7.88 mmol), sodium tert-butoxide (1.1 eq, 0.83 g, 96.1 g/mol, 8.67 mmol), tris(dibenzylideneacetone) dipalladium (0.05 eq, 0.36 g, 915 g/mol, 0.39 mmol), tri-tert-butylphosphine (0.05 eq, 0.079 g, 202.32 g/mol, 0.39 mmol) and toluene (40 ml) were added into a reaction flask, heated for reflux reaction for 5 h after the materials were added, cooled to room temperature after the reaction was finished, then added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 5 (3.81 g, yield 60.3%), ESI-MS (m/z) (M+): theoretical value 802.10, observed value 801.88.

### Example 2

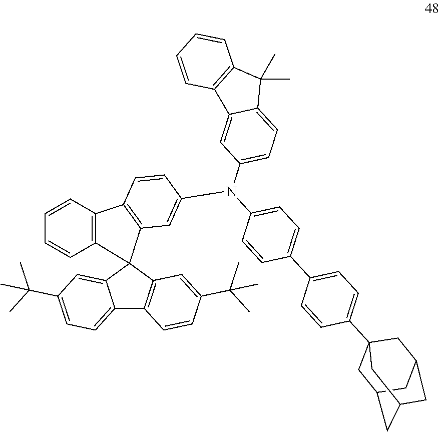 [SMILES: CC(C)(C)c1ccc2c(c1)C1(c3ccccc3-c3ccc(N(c4ccc(-c5ccc(C67CC8CC(CC(C8)C6)C7)cc5)cc4)c4ccc5c(c4)-c4ccccc4C5(C)C)cc31)c1cc(C(C)(C)C)ccc1-2]

A synthetic method of a novel organic electroluminescent compound 48 is as follows:

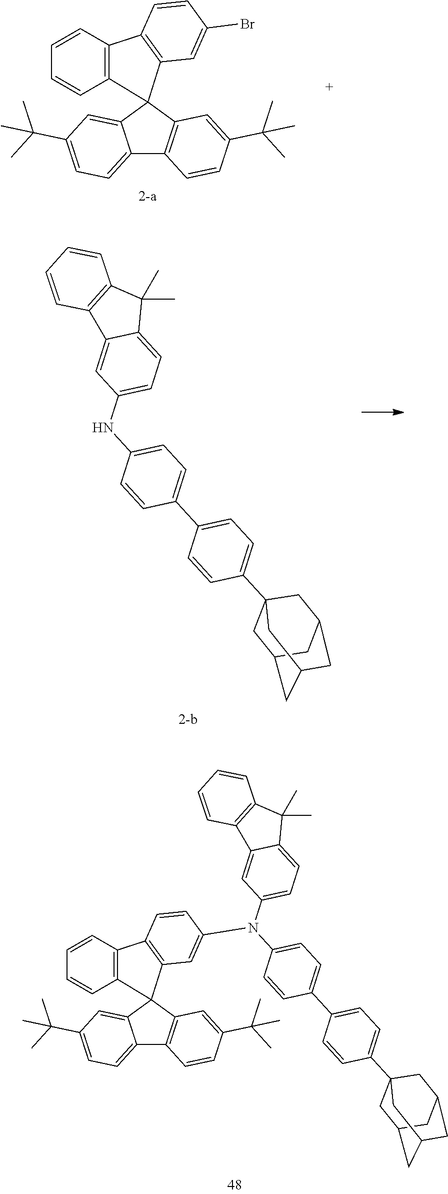

Under the protection of nitrogen, compound 2-a (4 g, 507.50 g/mol, 7.88 mmol), compound 2-b (1 eq, 3.91 g, 495.70 g/mol, 7.88 mmol), sodium tert-butoxide (1.1 eq, 0.83 g, 96.1 g/mol, 8.67 mmol), tris(dibenzylideneacetone) dipalladium (0.05 eq, 0.36 g, 915 g/mol, 0.39 mmol), tri-tert-butylphosphine (0.05 eq, 0.079 g, 202.32 g/mol, 0.39 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 48 (4.48 g, yield 61.7%), ESI-MS (m/z) (M+): theoretical value 922.29, observed value 921.88.

### Example 3

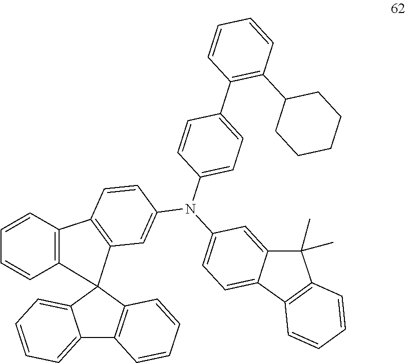 [SMILES: CC1(C)c2ccccc2-c2ccc(N(c3ccc(-c4ccccc4C4CCCCC4)cc3)c3ccc4c(c3)C3(c5ccccc5-c5ccccc53)c3ccccc3-4)cc21]

A synthetic method of a novel organic electroluminescent compound 62 is as follows:

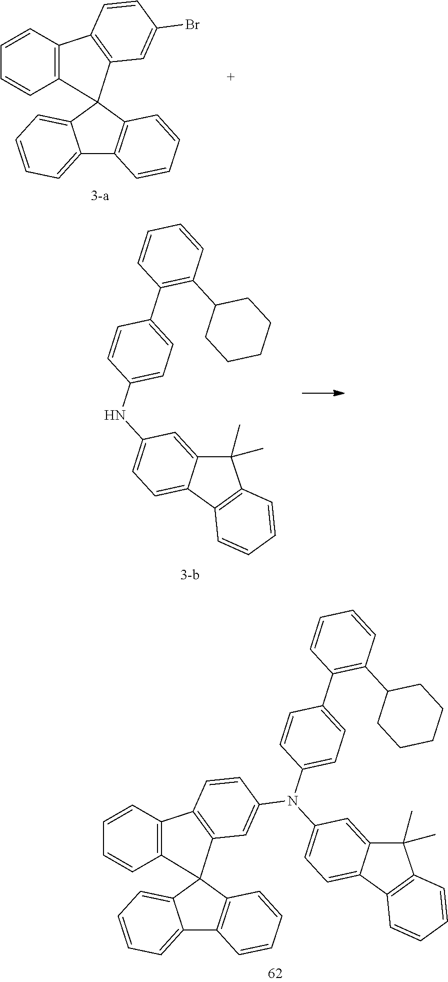 [SMILES: Brc1ccc2c(c1)C1(c3ccccc3-c3ccccc31)c1ccccc1-2 ; CC1(C)c2ccccc2-c2ccc(Nc3ccc(-c4ccccc4C4CCCCC4)cc3)cc21 ; CC1(C)c2ccccc2-c2ccc(N(c3ccc(-c4ccccc4C4CCCCC4)cc3)c3ccc4c(c3)C3(c5ccccc5-c5ccccc53)c3ccccc3-4)cc21]

Under the protection of nitrogen, compound 3-a (4 g, 395.29 g/mol, 10.12 mmol), compound 3-b (1 eq, 4.39 g, 433.62 g/mol, 10.12 mmol), sodium tert-butoxide (1.1 eq, 1.07 g, 96.1 g/mol, 11.13 mmol), tris(dibenzylideneacetone) dipalladium (0.05 eq, 0.46 g, 915 g/mol, 0.506 mmol), tri-tert-butylphosphine (0.05 eq, 0.102 g, 202.32 g/mol, 0.506 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 62 (4.84 g, yield 63.1%), ESI-MS (m/z) (M+): theoretical value 758.00, observed value 757.62.

### Example 4

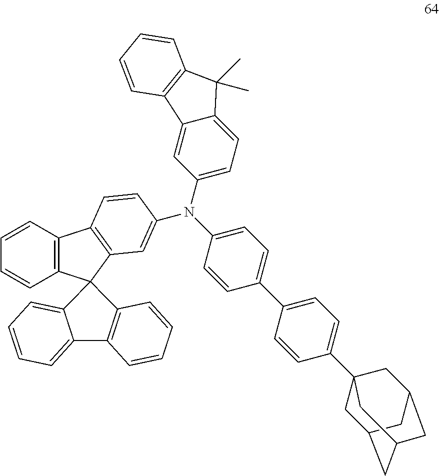 [SMILES: CC1(C)c2ccccc2-c2cc(N(c3ccc(-c4ccc(C56CC7CC(CC(C7)C5)C6)cc4)cc3)c3ccc4c(c3)C3(c5ccccc5-c5ccccc53)c3ccccc3-4)ccc21]

A synthetic method of a novel organic electroluminescent compound 64 is as follows:

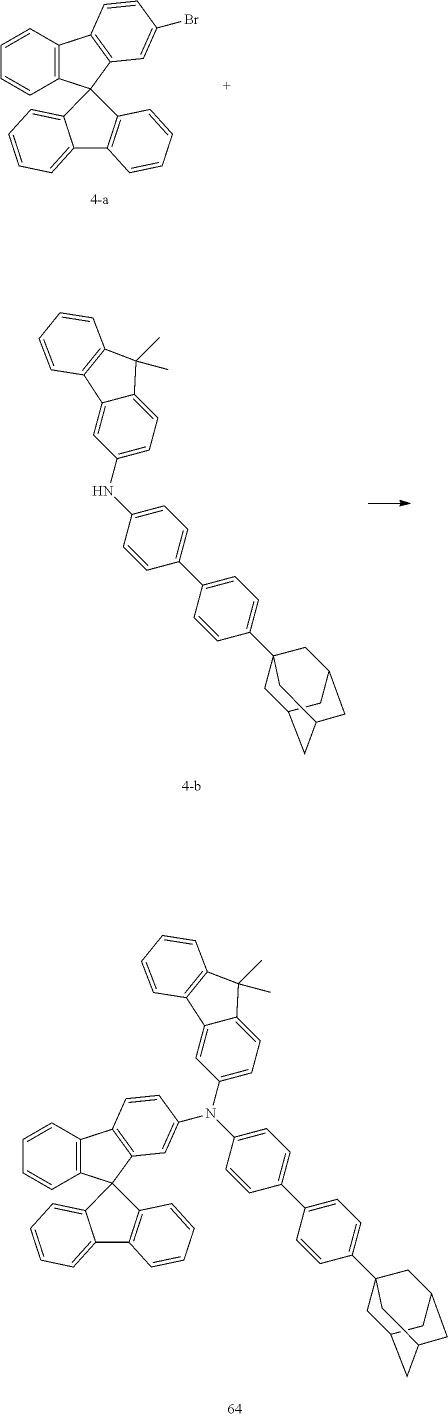 [SMILES: Brc1ccc2c(c1)C1(c3ccccc3-c3ccccc31)c1ccccc1-2 ; CC1(C)c2ccccc2-c2cc(Nc3ccc(-c4ccc(C56CC7CC(CC(C7)C5)C6)cc4)cc3)ccc21 ; CC1(C)c2ccccc2-c2cc(N(c3ccc(-c4ccc(C56CC7CC(CC(C7)C5)C6)cc4)cc3)c3ccc4c(c3)C3(c5ccccc5-c5ccccc53)c3ccccc3-4)ccc21]

Under the protection of nitrogen, compound 4-a (4 g, 395.29 g/mol, 10.12 mmol), compound 4-b (1 eq, 5.02 g, 495.70 g/mol, 10.12 mmol), sodium tert-butoxide (1.1 eq, 1.07 g, 96.1 g/mol, 11.13 mmol), tris(dibenzylideneacetone) dipalladium (0.05 eq, 0.46 g, 915 g/mol, 0.506 mmol), tri-tert-butylphosphine (0.05 eq, 0.102 g, 202.32 g/mol, 0.506 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 64 (5.14 g, yield 62.7%), ESI-MS (m/z) (M+): theoretical value 810.08, observed value 809.62.

### Example 5

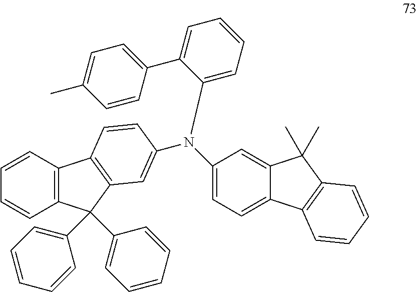 [SMILES: Cc1ccc(-c2ccccc2N(c2ccc3c(c2)C(C)(C)c2ccccc2-3)c2ccc3c(c2)C(c2ccccc2)(c2ccccc2)c2ccccc2-3)cc1]

A synthetic method of a novel organic electroluminescent compound 73 is as follows:

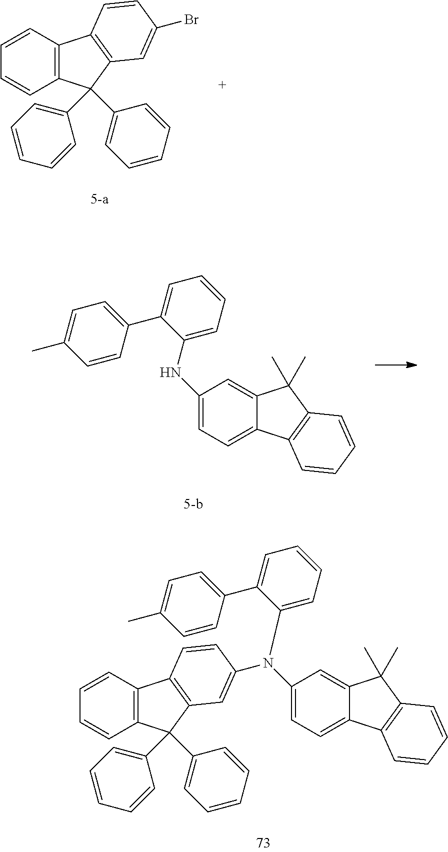 [SMILES: Brc1ccc2c(c1)C(c1ccccc1)(c1ccccc1)c1ccccc1-2 ; Cc1ccc(-c2ccccc2Nc2ccc3c(c2)C(C)(C)c2ccccc2-3)cc1 ; Cc1ccc(-c2ccccc2N(c2ccc3c(c2)C(C)(C)c2ccccc2-3)c2ccc3c(c2)C(c2ccccc2)(c2ccccc2)c2ccccc2-3)cc1]

Under the protection of nitrogen, compound 5-a (4 g, 397.31 g/mol, 10.07 mmol), compound 5-b (1 eq, 3.78 g, 375.50 g/mol, 10.07 mmol), sodium tert-butoxide (1.1 eq, 1.06 g, 96.1 g/mol, 11.07 mmol), tris(dibenzylideneacetone) dipalladium (0.05 eq, 0.46 g, 915 g/mol, 0.503 mmol), tri-tert-butylphosphine (0.05 eq, 0.102 g, 202.32 g/mol, 0.503 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 73 (4.28 g, yield 61.5%), ESI-MS (m/z) (M+): theoretical value 691.90, observed value 691.44.

### Example 6

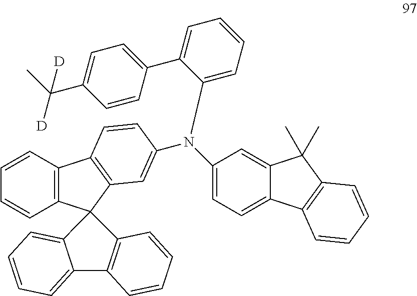 [SMILES: [2H]C([2H])(C)c1ccc(-c2ccccc2N(c2ccc3c(c2)C(C)(C)c2ccccc2-3)c2ccc3c(c2)C2(c4ccccc4-c4ccccc42)c2ccccc2-3)cc1]

A synthetic method of a novel organic electroluminescent compound 97 is as follows:

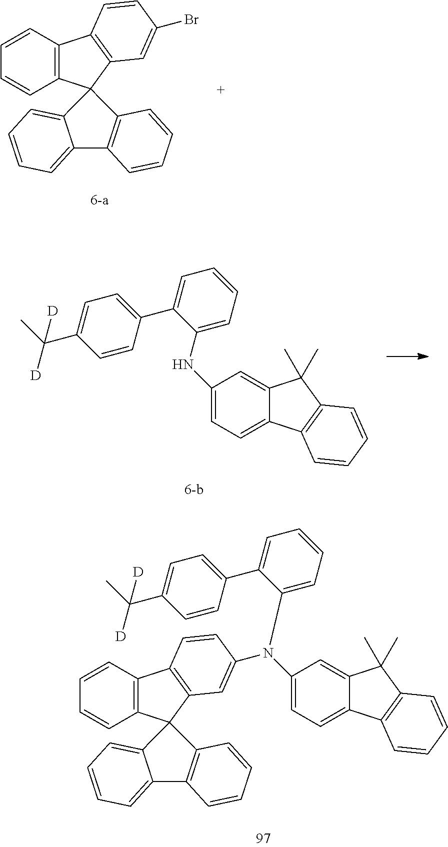 [SMILES: Brc1ccc2c(c1)C1(c3ccccc3-c3ccccc31)c1ccccc1-2 ; [2H]C([2H])(C)c1ccc(-c2ccccc2Nc2ccc3c(c2)C(C)(C)c2ccccc2-3)cc1 ; [2H]C([2H])(C)c1ccc(-c2ccccc2N(c2ccc3c(c2)C(C)(C)c2ccccc2-3)c2ccc3c(c2)C2(c4ccccc4-c4ccccc42)c2ccccc2-3)cc1]

Under the protection of nitrogen, compound 6-a (4 g, 395.29 g/mol, 10.12 mmol), compound 6-b (1 eq, 3.96 g, 391.54 g/mol, 10.12 mmol), sodium tert-butoxide (1.1 eq, 1.07 g, 96.1 g/mol, 11.13 mmol), tris(dibenzylideneacetone) dipalladium (0.05 eq, 0.46 g, 915 g/mol, 0.506 mmol), tri-tert-butylphosphine (0.05 eq, 0.102 g, 202.32 g/mol, 0.506 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 97 (4.56 g, yield 63.8%), ESI-MS (m/z) (M+): theoretical value 705.92, observed value 705.52.

### Example 7

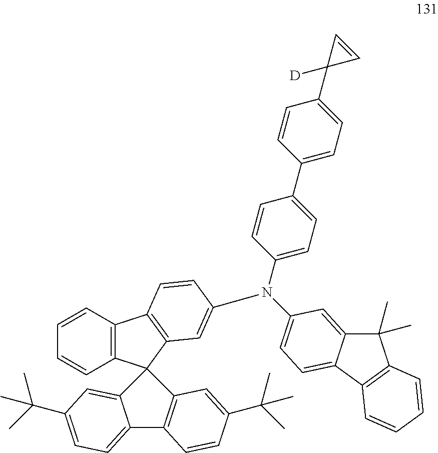 [SMILES: [2H]C1(c2ccc(-c3ccc(N(c4ccc5c(c4)C(C)(C)c4ccccc4-5)c4ccc5c(c4)C4(c6ccccc6-5)c5cc(C(C)(C)C)ccc5-c5ccc(C(C)(C)C)cc54)cc3)cc2)C=C1]

A synthetic method of a novel organic electroluminescent compound 131 is as follows:

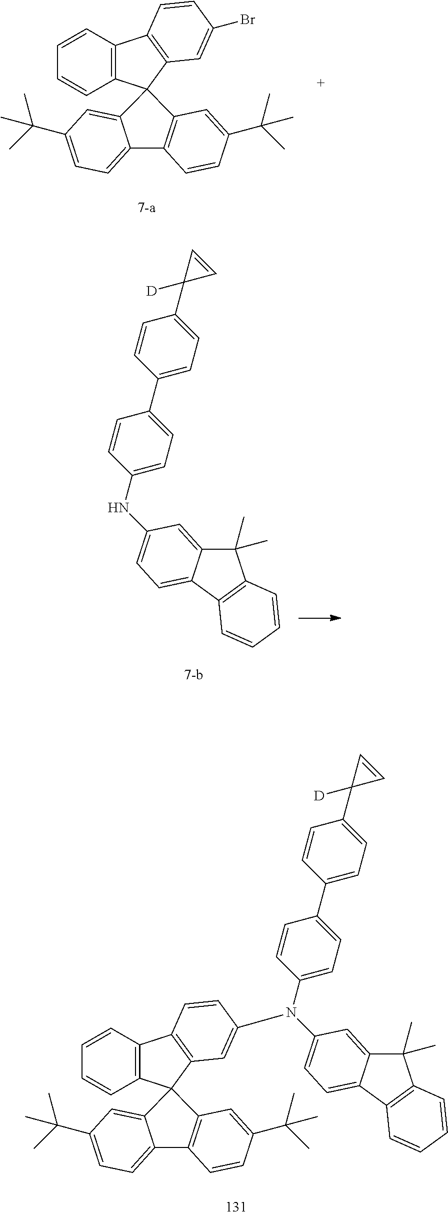

Under the protection of nitrogen, compound 7-a (4 g, 507.50 g/mol, 7.88 mmol), compound 7-b (1 eq, 3.16 g, 400.53 g/mol, 7.88 mmol), sodium tert-butoxide (1.1 eq, 0.83 g, 96.1 g/mol, 8.67 mmol), tris(dibenzylideneacetone) dipalladium (0.05 eq, 0.36 g, 915 g/mol, 0.39 mmol), tri-tert-butylphosphine (0.05 eq, 0.079 g, 202.32 g/mol, 0.39 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 131 (4.24 g, yield 65.1%), ESI-MS (m/z) (M+): theoretical value 827.12, observed value 827.03.

### Example 8

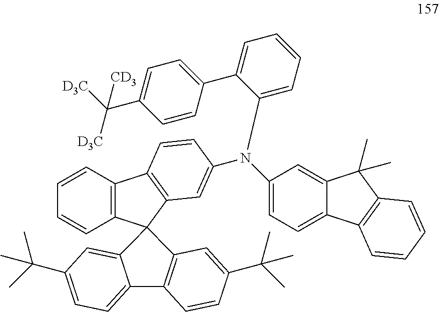 [SMILES: [2H]C([2H])([2H])C(c1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1)(C([2H])([2H])[2H])C([2H])([2H])[2H]]

A synthetic method of a novel organic electroluminescent compound 157 is as follows:

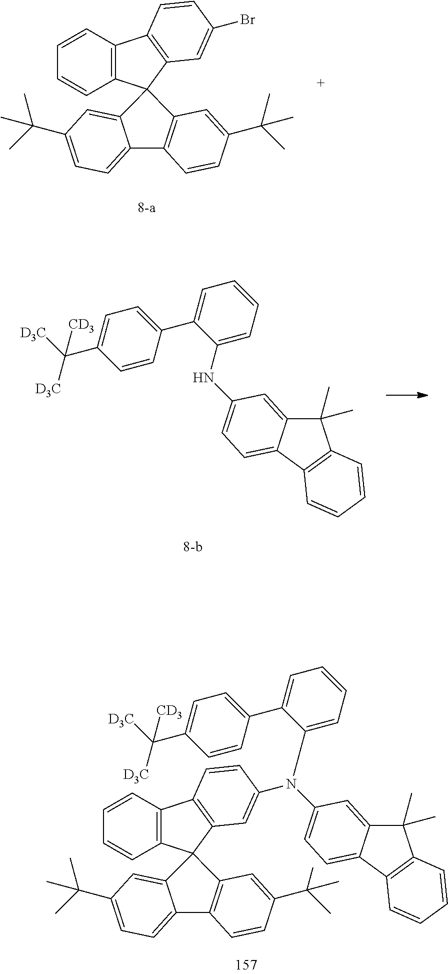

Under the protection of nitrogen, compound 8-a (4 g, 507.50 g/mol, 7.88 mmol), compound 8-b (1 eq, 3.36 g, 426.64 g/mol, 7.88 mmol), sodium tert-butoxide (1.1 eq, 0.83 g, 96.1 g/mol, 8.67 mmol), tris(dibenzylideneacetone) dipalladium (0.05 eq, 0.36 g, 915 g/mol, 0.39 mmol), tri-tert-butylphosphine (0.05 eq, 0.079 g, 202.32 g/mol, 0.39 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 157 (4.22 g, yield 62.8%), ESI-MS (m/z) (M+): theoretical value 853.23, observed value 853.07.

### Example 9

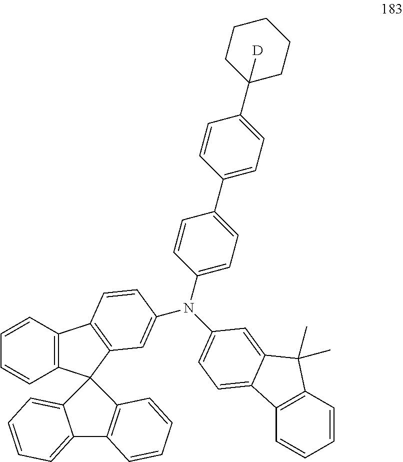

A synthetic method of a novel organic electroluminescent compound 183 is as follows:

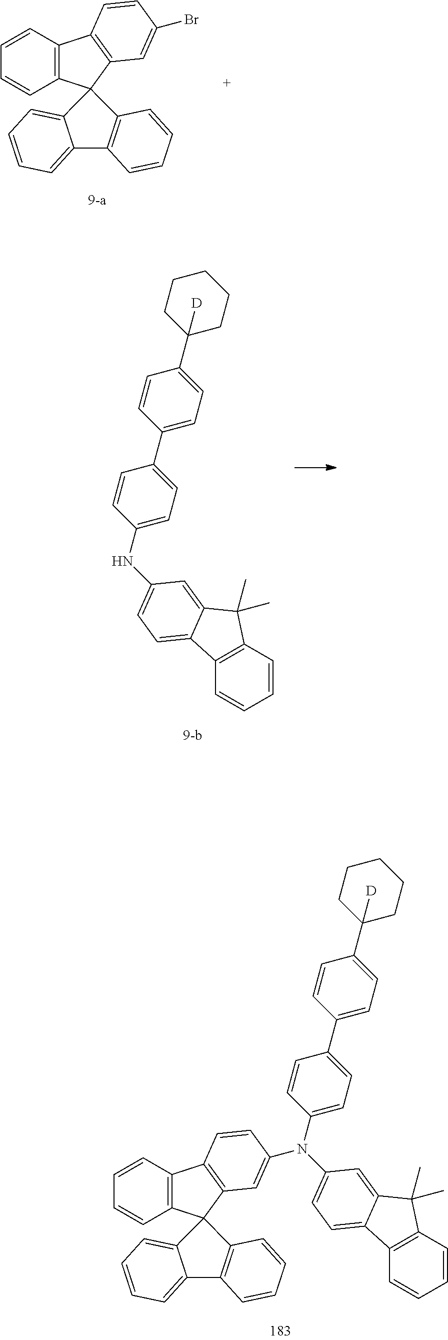

Under the protection of nitrogen, compound 9-a (4 g, 395.29 g/mol, 10.12 mmol), compound 9-b (1 eq, 4.50 g, 444.63 g/mol, 10.12 mmol), sodium tert-butoxide (1.1 eq, 1.07 g, 96.1 g/mol, 11.13 mmol), tris(dibenzylideneacetone) dipalladium (0.05 eq, 0.46 g, 915 g/mol, 0.506 mmol), tri-tert-butylphosphine (0.05 eq, 0.102 g, 202.32 g/mol, 0.506 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 183 (4.69 g, yield 61.1%), ESI-MS (m/z) (M+): theoretical value 759.01, observed value 705.08.

### Example 10

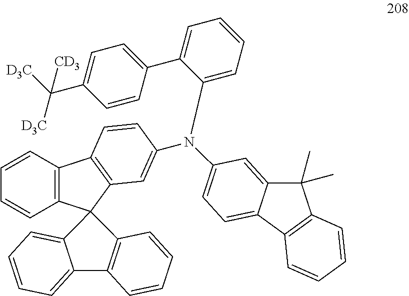 [SMILES: [2H]C([2H])([2H])C(c1ccc(-c2ccccc2N(c2ccc3c(c2)C(C)(C)c2ccccc2-3)c2ccc3c(c2)C2(c4ccccc4-c4ccccc42)c2ccccc2-3)cc1)(C([2H])([2H])[2H])C([2H])([2H])[2H]]

A synthetic method of a novel organic electroluminescent compound 208 is as follows:

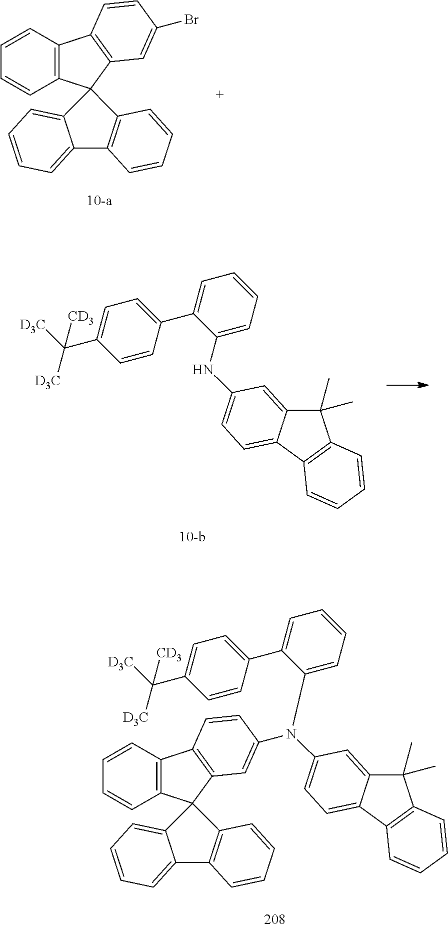 [SMILES: Brc1ccc2c(c1)C1(c3ccccc3-c3ccccc31)c1ccccc1-2 ; [2H]C([2H])([2H])C(c1ccc(-c2ccccc2Nc2ccc3c(c2)C(C)(C)c2ccccc2-3)cc1)(C([2H])([2H])[2H])C([2H])([2H])[2H] ; [2H]C([2H])([2H])C(c1ccc(-c2ccccc2N(c2ccc3c(c2)C(C)(C)c2ccccc2-3)c2ccc3c(c2)C2(c4ccccc4-c4ccccc42)c2ccccc2-3)cc1)(C([2H])([2H])[2H])C([2H])([2H])[2H]]

Under the protection of nitrogen, compound 10-a (4 g, 395.29 g/mol, 10.12 mmol), compound 10-b (1 eq, 4.32 g, 426.64 g/mol, 10.12 mmol), sodium tert-butoxide (1.1 eq, 1.07 g, 96.1 g/mol, 11.13 mmol), tris(dibenzylideneacetone) dipalladium (0.05 eq, 0.46 g, 915 g/mol, 0.506 mmol), tri-tert-butylphosphine (0.05 eq, 0.102 g, 202.32 g/mol, 0.506 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 208 (4.63 g, yield 61.7%), ESI-MS (m/z) (M+): theoretical value 741.02, observed value 741.14.

### Example 11

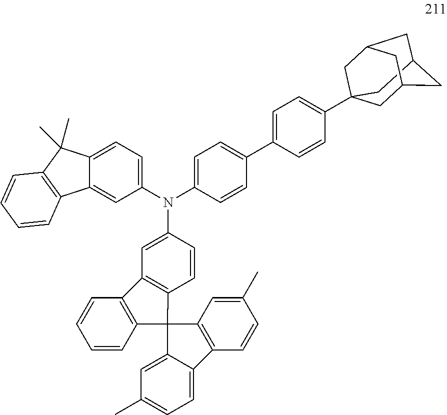 [SMILES: Cc1ccc2c(c1)C1(c3ccccc3-c3cc(N(c4ccc(-c5ccc(C67CC8CC(CC(C8)C6)C7)cc5)cc4)c4ccc5c(c4)-c4ccccc4C5(C)C)ccc31)c1cc(C)ccc1-2]

A synthetic method of a novel organic electroluminescent compound 211 is as follows:

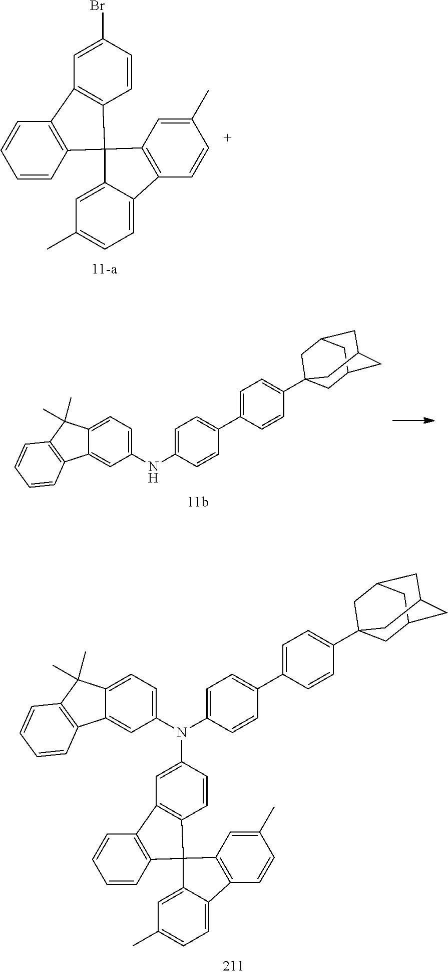 [SMILES: Cc1ccc2c(c1)C1(c3ccccc3-c3cc(Br)ccc31)c1cc(C)ccc1-2 ; CC1(C)c2ccccc2-c2cc(Nc3ccc(-c4ccc(C56CC7CC(CC(C7)C5)C6)cc4)cc3)ccc21 ; Cc1ccc2c(c1)C1(c3ccccc3-c3cc(N(c4ccc(-c5ccc(C67CC8CC(CC(C8)C6)C7)cc5)cc4)c4ccc5c(c4)-c4ccccc4C5(C)C)ccc31)c1cc(C)ccc1-2]

Under the protection of nitrogen, compound 11-a (4 g, 423.34 g/mol, 9.45 mmol), compound 11-b (1 eq, 4.68 g, 495.70 g/mol, 9.45 mmol), sodium tert-butoxide (1.1 eq, 1.00 g, 96.1 g/mol, 10.39 mmol), tris(dibenzylideneacetone) dipalladium (0.05 eq, 0.43 g, 915 g/mol, 0.472 mmol), tri-tert-butylphosphine (0.05 eq, 0.096 g, 202.32 g/mol, 0.472 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 211 (4.80 g, yield 60.6%), ESI-MS (m/z) (M+): theoretical value 838.13, observed value 837.86.

### Example 12

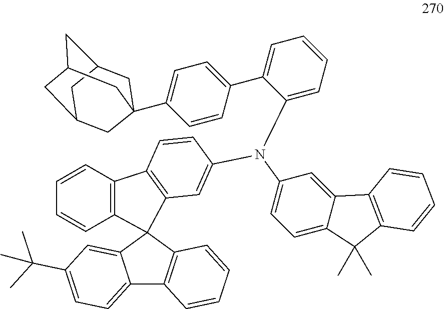 [SMILES: CC(C)(C)c1ccc2c(c1)C1(c3ccccc3-c3ccc(N(c4ccc5c(c4)-c4ccccc4C5(C)C)c4ccccc4-c4ccc(C56CC7CC(CC(C7)C5)C6)cc4)cc31)c1ccccc1-2]

A synthetic method of a novel organic electroluminescent compound 270 is as follows:

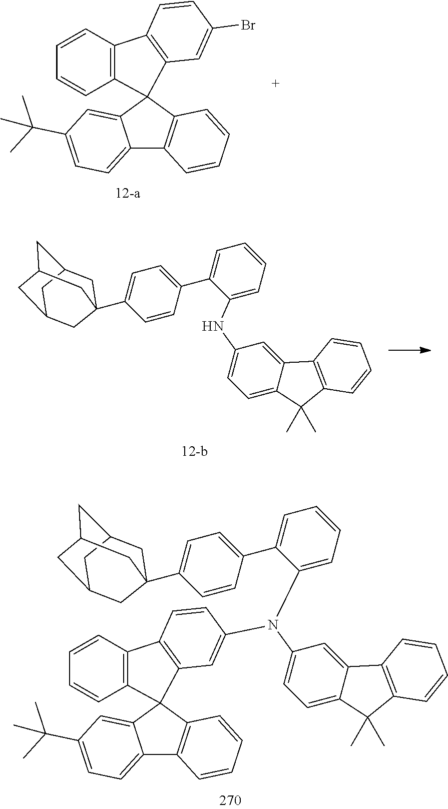 [SMILES: CC(C)(C)c1ccc2c(c1)C1(c3ccccc3-c3ccc(Br)cc31)c1ccccc1-2 ; CC1(C)c2ccccc2-c2cc(Nc3ccccc3-c3ccc(C45CC6CC(CC(C6)C4)C5)cc3)ccc21 ; CC(C)(C)c1ccc2c(c1)C1(c3ccccc3-c3ccc(N(c4ccc5c(c4)-c4ccccc4C5(C)C)c4ccccc4-c4ccc(C56CC7CC(CC(C7)C5)C6)cc4)cc31)c1ccccc1-2]

Under the protection of nitrogen, compound 12-a (4 g, 451.40 g/mol, 8.86 mmol), compound 12-b (1 eq, 4.39 g, 495.70 g/mol, 8.86 mmol), sodium tert-butoxide (1.1 eq, 0.94 g, 96.1 g/mol, 9.75 mmol), tris(dibenzylideneacetone) dipalladium (0.05 eq, 0.41 g, 915 g/mol, 0.443 mmol), tri-tert-butylphosphine (0.05 eq, 0.090 g, 202.32 g/mol, 0.443 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 270 (4.81 g, yield 62.7%), ESI-MS (m/z) (M+): theoretical value 866.18, observed value 865.82.

### Example 13

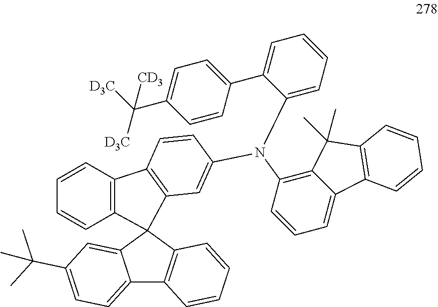 [SMILES: [2H]C([2H])([2H])C(c1ccc(-c2ccccc2N(c2ccc3c(c2)C2(c4ccccc4-3)c3ccccc3-c3ccc(C(C)(C)C)cc32)c2cccc3c2C(C)(C)c2ccccc2-3)cc1)(C([2H])([2H])[2H])C([2H])([2H])[2H]]

A synthetic method of a novel organic electroluminescent compound 278 is as follows:

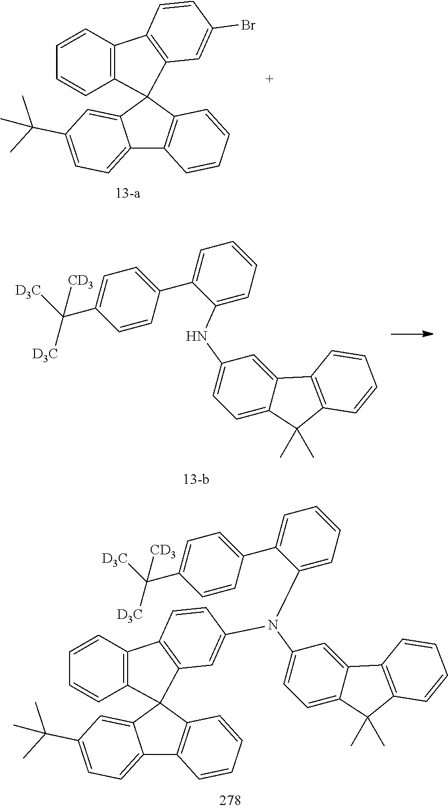 [SMILES: CC(C)(C)c1ccc2c(c1)C1(c3ccccc3-c3ccc(Br)cc31)c1ccccc1-2 ; [2H]C([2H])([2H])C(c1ccc(-c2ccccc2Nc2ccc3c(c2)-c2ccccc2C3(C)C)cc1)(C([2H])([2H])[2H])C([2H])([2H])[2H] ; [2H]C([2H])([2H])C(c1ccc(-c2ccccc2N(c2ccc3c(c2)-c2ccccc2C3(C)C)c2ccc3c(c2)C2(c4ccccc4-3)c3ccccc3-c3ccc(C(C)(C)C)cc32)cc1)(C([2H])([2H])[2H])C([2H])([2H])[2H]]

Under the protection of nitrogen, compound 13-a (4 g, 451.40 g/mol, 8.86 mmol), compound 13-b (1 eq, 3.78 g, 426.64 g/mol, 8.86 mmol), sodium tert-butoxide (1.1 eq, 0.94 g, 96.1 g/mol, 9.75 mmol), tris(dibenzylideneacetone) dipalladium (0.05 eq, 0.41 g, 915 g/mol, 0.443 mmol), tri-tert-butylphosphine (0.05 eq, 0.090 g, 202.32 g/mol, 0.443 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 278 (4.39 g, yield 62.1%), ESI-MS (m/z) (M+): theoretical value 797.12, observed value 797.05.

### Example 14

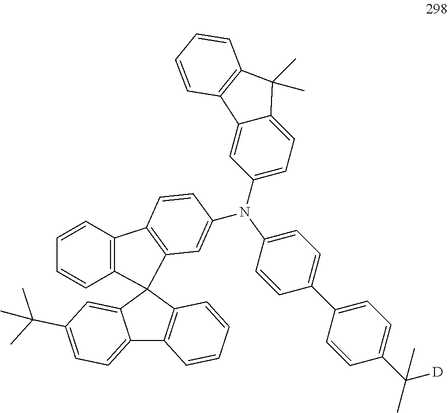 [SMILES: [2H]C(C)(C)c1ccc(-c2ccc(N(c3ccc4c(c3)-c3ccccc3C4(C)C)c3ccc4c(c3)C3(c5ccccc5-4)c4ccccc4-c4ccc(C(C)(C)C)cc43)cc2)cc1]

A synthetic method of a novel organic electroluminescent compound 298 is as follows:

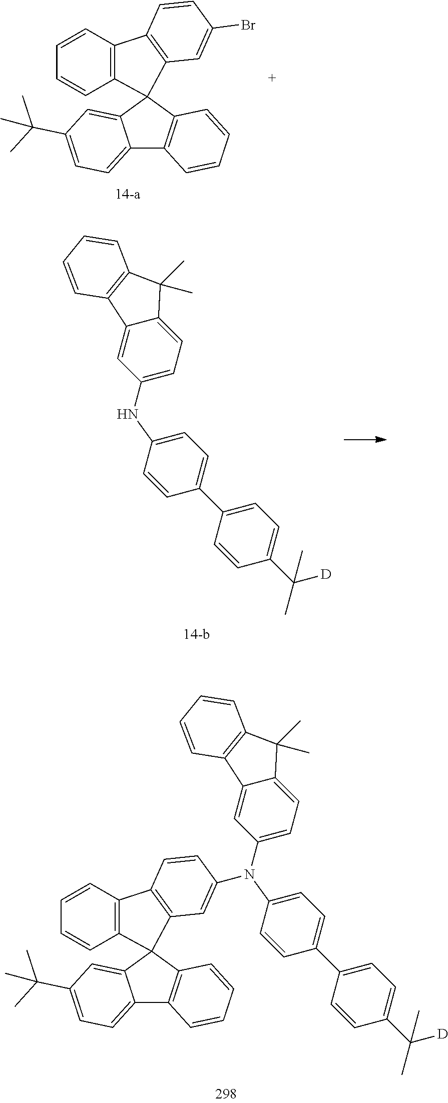 [SMILES: CC(C)(C)c1ccc2c(c1)C1(c3ccccc3-c3ccc(Br)cc31)c1ccccc1-2 ; [2H]C(C)(C)c1ccc(-c2ccc(Nc3ccc4c(c3)-c3ccccc3C4(C)C)cc2)cc1 ; [2H]C(C)(C)c1ccc(-c2ccc(N(c3ccc4c(c3)-c3ccccc3C4(C)C)c3ccc4c(c3)C3(c5ccccc5-4)c4ccccc4-c4ccc(C(C)(C)C)cc43)cc2)cc1]

Under the protection of nitrogen, compound 14-a (4 g, 451.40 g/mol, 8.86 mmol), compound 14-b (1 eq, 3.58 g, 404.56 g/mol, 8.86 mmol), sodium tert-butoxide (1.1 eq, 0.94 g, 96.1 g/mol, 9.75 mmol), tris(dibenzylideneacetone) dipalladium (0.05 eq, 0.41 g, 915 g/mol, 0.443 mmol), tri-tert-butylphosphine (0.05 eq, 0.090 g, 202.32 g/mol, 0.443 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 298 (4.53 g, yield 65.9%), ESI-MS (m/z) (M+): theoretical value 775.05, observed value 775.11.

### Example 15

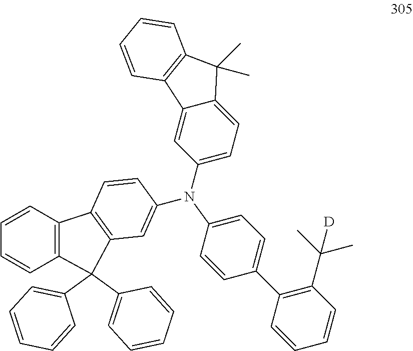 [SMILES: [2H]C(C)(C)c1ccccc1-c1ccc(N(c2ccc3c(c2)-c2ccccc2C3(C)C)c2ccc3c(c2)C(c2ccccc2)(c2ccccc2)c2ccccc2-3)cc1]

A synthetic method of a novel organic electroluminescent compound 305 is as follows:

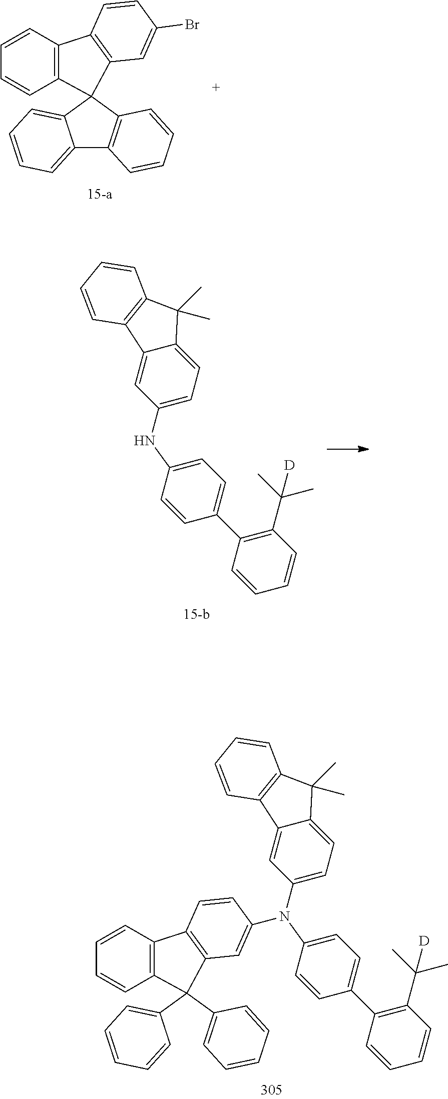 [SMILES: Brc1ccc2c(c1)C1(c3ccccc3-c3ccccc31)c1ccccc1-2 ; [2H]C(C)(C)c1ccccc1-c1ccc(Nc2ccc3c(c2)-c2ccccc2C3(C)C)cc1 ; [2H]C(C)(C)c1ccccc1-c1ccc(N(c2ccc3c(c2)-c2ccccc2C3(C)C)c2ccc3c(c2)C(c2ccccc2)(c2ccccc2)c2ccccc2-3)cc1]

Under the protection of nitrogen, compound 305-a (4 g, 397.31 g/mol, 10.07 mmol), compound 305-b (1 eq, 4.07 g, 404.56 g/mol, 10.07 mmol), sodium tert-butoxide (1.1 eq, 1.06 g, 96.1 g/mol, 11.07 mmol), tris(dibenzylideneacetone) dipalladium (0.05 eq, 0.46 g, 915 g/mol, 0.503 mmol), tri-tert-butylphosphine (0.05 eq, 0.102 g, 202.32 g/mol, 0.503 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 305 (4.46 g, yield 61.4%), ESI-MS (m/z) (M+): theoretical value 720.96, observed value 719.87.

### Example 16

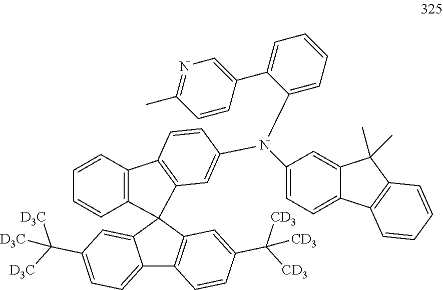 [SMILES: [2H]C([2H])([2H])C(c1ccc2c(c1)C1(c3ccccc3-c3ccc(N(c4ccc5c(c4)C(C)(C)c4ccccc4-5)c4ccccc4-c4ccc(C)nc4)cc31)c1cc(C(C([2H])([2H])[2H])(C([2H])([2H])[2H])C([2H])([2H])[2H])ccc1-2)(C([2H])([2H])[2H])C([2H])([2H])[2H]]

A synthetic method of a novel organic electroluminescent compound 325 is as follows:

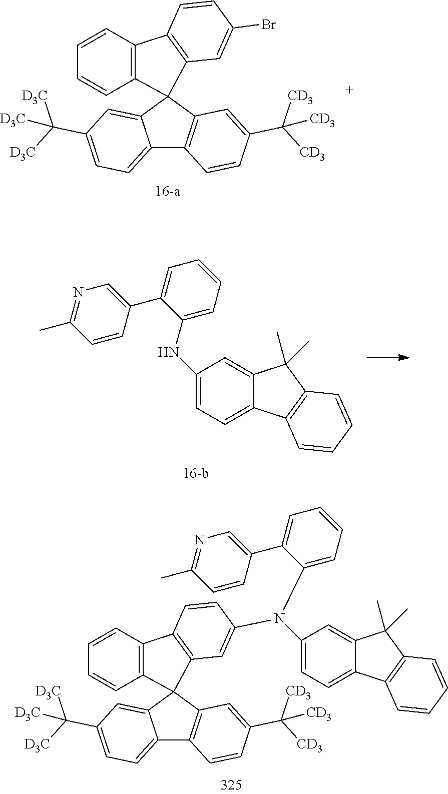 [SMILES: [2H]C([2H])([2H])C(c1ccc2c(c1)C1(c3ccccc3-c3ccc(Br)cc31)c1cc(C(C([2H])([2H])[2H])(C([2H])([2H])[2H])C([2H])([2H])[2H])ccc1-2)(C([2H])([2H])[2H])C([2H])([2H])[2H] ; Cc1ccc(-c2ccccc2Nc2ccc3c(c2)C(C)(C)c2ccccc2-3)cn1 ; [2H]C([2H])([2H])C(c1ccc2c(c1)C1(c3ccccc3-c3ccc(N(c4ccc5c(c4)C(C)(C)c4ccccc4-5)c4ccccc4-c4ccc(C)nc4)cc31)c1cc(C(C([2H])([2H])[2H])(C([2H])([2H])[2H])C([2H])([2H])[2H])ccc1-2)(C([2H])([2H])[2H])C([2H])([2H])[2H]]

Under the protection of nitrogen, compound 16-a (4 g, 525.61 g/mol, 7.61 mmol), compound 16-b (1 eq, 2.87 g, 376.49 g/mol, 7.61 mmol), sodium tert-butoxide (1.1 eq, 0.804 g, 96.1 g/mol, 8.37 mmol), tris(dibenzylideneacetone) dipalladium (0.05 eq, 0.35 g, 915 g/mol, 0.381 mmol), tri-tert-butylphosphine (0.05 eq, 0.077 g, 202.32 g/mol, 0.381 mmol) and toluene (40 ml) were added into a reaction flask, after the addition was finished, the mixture was heated for reflux reaction for 5 h, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 325 (4.14 g, yield 66.3%), ESI-MS (m/z) (M+): theoretical value 821.20, observed value 821.05.

### Example 17

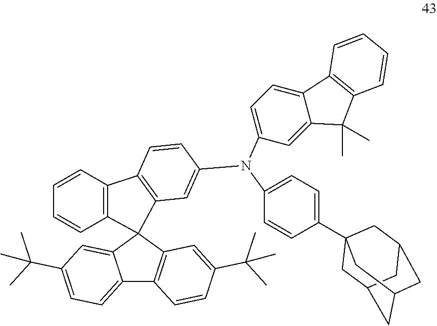 [SMILES: CC(C)(C)c1ccc2c(c1)C1(c3ccccc3-c3ccc(N(c4ccc(C56CC7CC(CC(C7)C5)C6)cc4)c4ccc5c(c4)C(C)(C)c4ccccc4-5)cc31)c1cc(C(C)(C)C)ccc1-2]

A synthetic method of a novel organic electroluminescent compound 43 is as follows:

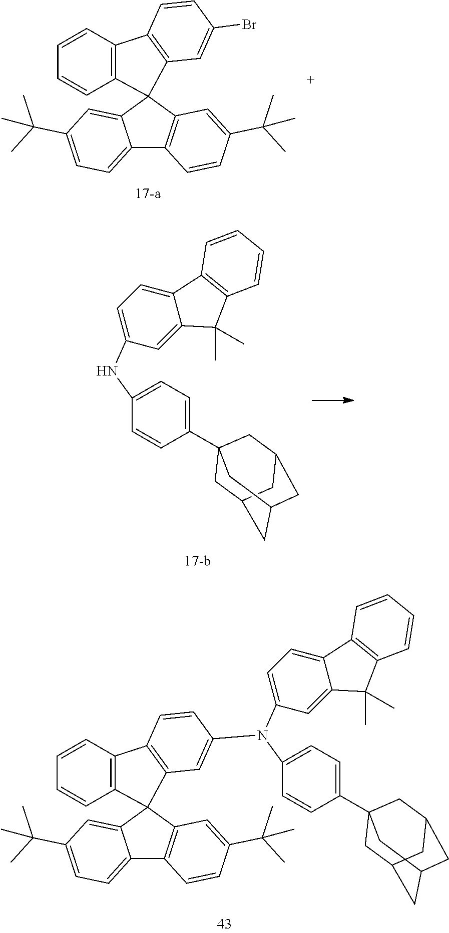

Under the protection of nitrogen, compound 17-a (4 g, 507.50 g/mol, 7.88 mmol), compound 17-b (1 eq, 3.31 g, 419.60 g/mol, 7.88 mmol), sodium tert-butoxide (1.1 eq, 0.83 g, 96.1 g/mol, 8.67 mmol), tris(dibenzylideneacetone) dipalladium (0.05 eq, 0.36 g, 915 g/mol, 0.39 mmol), tri-tert-butylphosphine (0.05 eq, 0.079 g, 202.32 g/mol, 0.39 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 43 (4.15 g, yield 62.2%), ESI-MS (m/z) (M+): theoretical value 846.19, observed value 845.88.

### Example 18

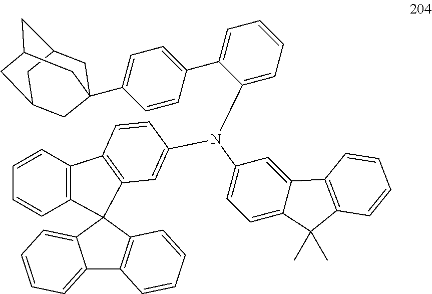 [SMILES: CC1(C)c2ccccc2-c2cc(N(c3ccc4c(c3)C3(c5ccccc5-c5ccccc53)c3ccccc3-4)c3ccccc3-c3ccc(C45CC6CC(CC(C6)C4)C5)cc3)ccc21]

A synthetic method of a novel organic electroluminescent compound 204 is as follows:

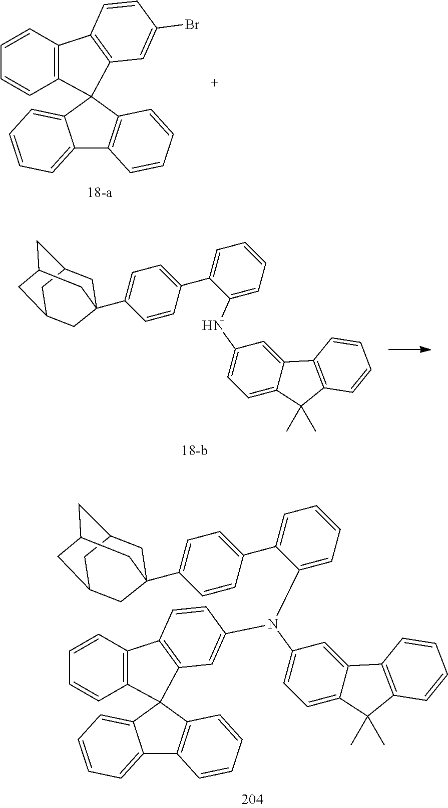 [SMILES: Brc1ccc2c(c1)C1(c3ccccc3-c3ccccc31)c1ccccc1-2 ; CC1(C)c2ccccc2-c2cc(Nc3ccccc3-c3ccc(C45CC6CC(CC(C6)C4)C5)cc3)ccc21 ; CC1(C)c2ccccc2-c2cc(N(c3ccc4c(c3)C3(c5ccccc5-c5ccccc53)c3ccccc3-4)c3ccccc3-c3ccc(C45CC6CC(CC(C6)C4)C5)cc3)ccc21]

Under the protection of nitrogen, compound 18-a (4 g, 395.29 g/mol, 10.12 mmol), compound 18-b (1 eq, 5.02 g, 495.70 g/mol, 10.12 mmol), sodium tert-butoxide (1.1 eq, 1.07 g, 96.1 g/mol, 11.13 mmol), tris(dibenzylideneacetone) dipalladium (0.05 eq, 0.46 g, 915 g/mol, 0.506 mmol), tri-tert-butylphosphine (0.05 eq, 0.102 g, 202.32 g/mol, 0.506 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 204 (5.07 g, yield 61.9%), ESI-MS (m/z) (M+): theoretical value 810.08, observed value 809.64.

### Example 19

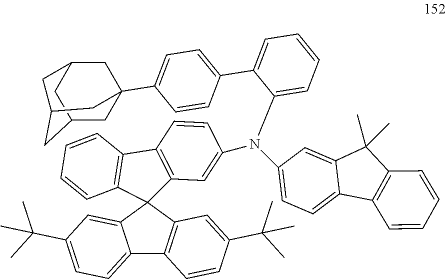 [SMILES: CC(C)(C)c1ccc2c(c1)C1(c3ccccc3-c3ccc(N(c4ccc5c(c4)C(C)(C)c4ccccc4-5)c4ccccc4-c4ccc(C56CC7CC(CC(C7)C5)C6)cc4)cc31)c1cc(C(C)(C)C)ccc1-2]

A synthetic method of a novel organic electroluminescent compound 152 is as follows:

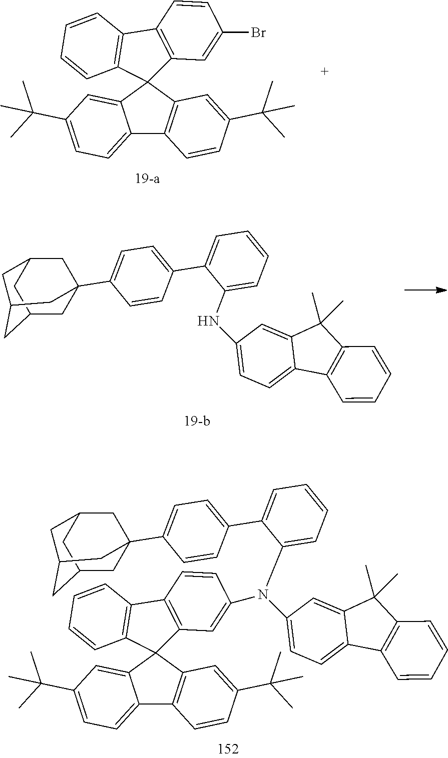

Under the protection of nitrogen, compound 19-a (4 g, 507.50 g/mol, 7.88 mmol), compound 19-b (1 eq, 3.91 g, 495.70 g/mol, 7.88 mmol), sodium tert-butoxide (1.1 eq, 0.83 g, 96.1 g/mol, 8.67 mmol), tris(dibenzylideneacetone) dipalladium (0.05 eq, 0.36 g, 915 g/mol, 0.39 mmol), tri-tert-butylphosphine (0.05 eq, 0.079 g, 202.32 g/mol, 0.39 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 152 (4.43 g, yield 60.9%), ESI-MS (m/z) (M+): theoretical value 922.29, observed value 921.79.

### Example 20

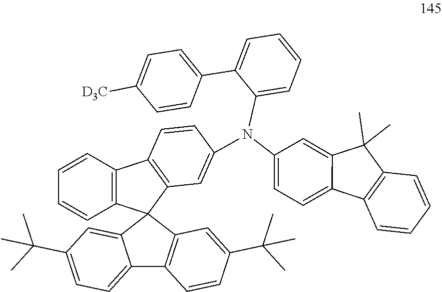 [SMILES: [2H]C([2H])([2H])c1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1]

A synthetic method of a novel organic electroluminescent compound 145 is as follows:

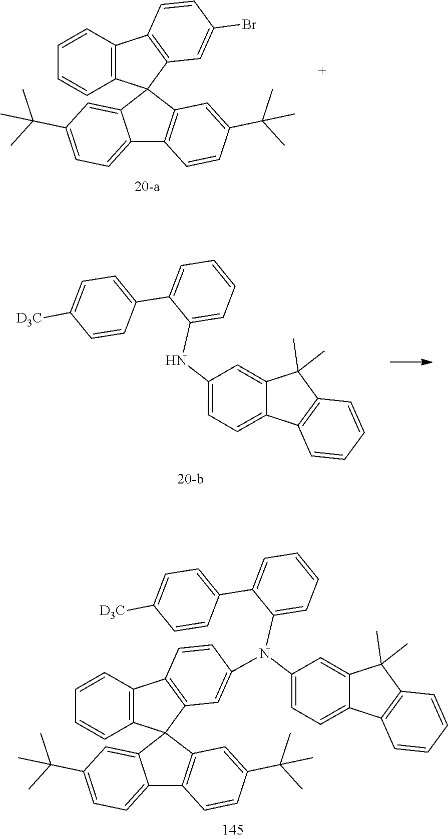

Under the protection of nitrogen, compound 20-a (4 g, 507.50 g/mol, 7.88 mmol), compound 20-b (1 eq, 2.98 g, 378.52 g/mol, 7.88 mmol), sodium tert-butoxide (1.1 eq, 0.83 g, 96.1 g/mol, 8.67 mmol), tris(dibenzylideneacetone) dipalladium (0.05 eq, 0.36 g, 915 g/mol, 0.39 mmol), tri-tert-butylphosphine (0.05 eq, 0.079 g, 202.32 g/mol, 0.39 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 145 (4.09 g, yield 64.5%), ESI-MS (m/z) (M+): theoretical value 805.11, observed value 805.36.

### Example 21

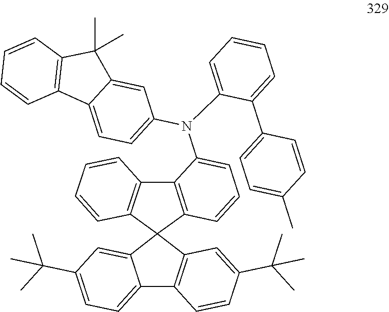 [SMILES: Cc1ccc(-c2ccccc2N(c2ccc3c(c2)C(C)(C)c2ccccc2-3)c2cccc3c2-c2ccccc2C32c3cc(C(C)(C)C)ccc3-c3ccc(C(C)(C)C)cc32)cc1]

A synthetic method of a novel organic electroluminescent compound 329 is as follows:

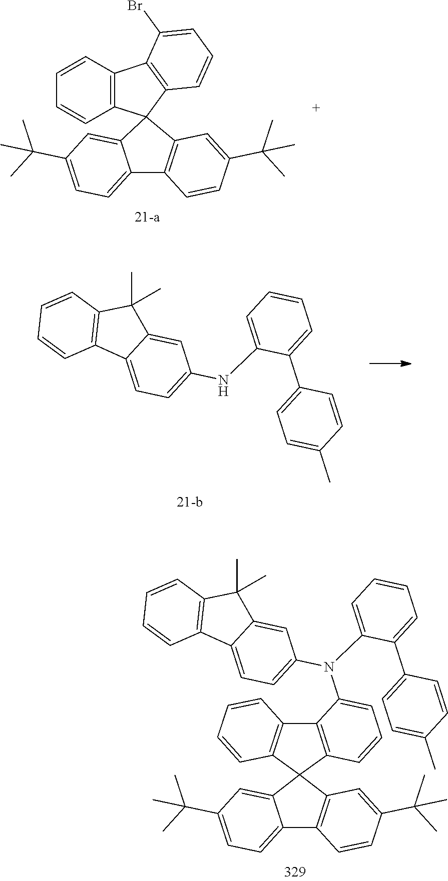 [SMILES: CC(C)(C)c1ccc2c(c1)C1(c3cc(C(C)(C)C)ccc3-2)c2ccccc2-c2c(Br)cccc21 ; Cc1ccc(-c2ccccc2Nc2ccc3c(c2)C(C)(C)c2ccccc2-3)cc1 ; Cc1ccc(-c2ccccc2N(c2ccc3c(c2)C(C)(C)c2ccccc2-3)c2cccc3c2-c2ccccc2C32c3cc(C(C)(C)C)ccc3-c3ccc(C(C)(C)C)cc32)cc1]

Under the protection of nitrogen, compound 21-a (4 g, 507.50 g/mol, 7.88 mmol), compound 21-b (1 eq, 2.96 g, 375.50 g/mol, 7.88 mmol), sodium tert-butoxide (1.1 eq, 0.83 g, 96.1 g/mol, 8.67 mmol), tris(dibenzylideneacetone) dipalladium (0.05 eq, 0.36 g, 915 g/mol, 0.39 mmol), tri-tert-butylphosphine (0.05 eq, 0.079 g, 202.32 g/mol, 0.39 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 329 (3.68 g, yield 58.3%), ESI-MS (m/z) (M+): theoretical value 802.10, observed value 801.86.

### Example 22

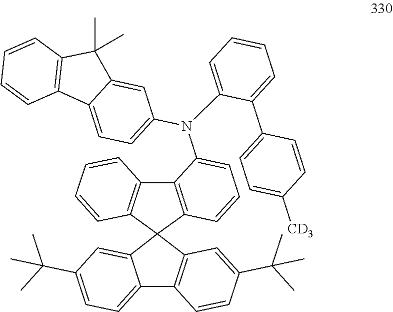 [SMILES: [2H]C([2H])([2H])c1ccc(-c2ccccc2N(c2ccc3c(c2)C(C)(C)c2ccccc2-3)c2cccc3c2-c2ccccc2C32c3cc(C(C)(C)C)ccc3-c3ccc(C(C)(C)C)cc32)cc1]

A synthetic method of a novel organic electroluminescent compound 330 is as follows:

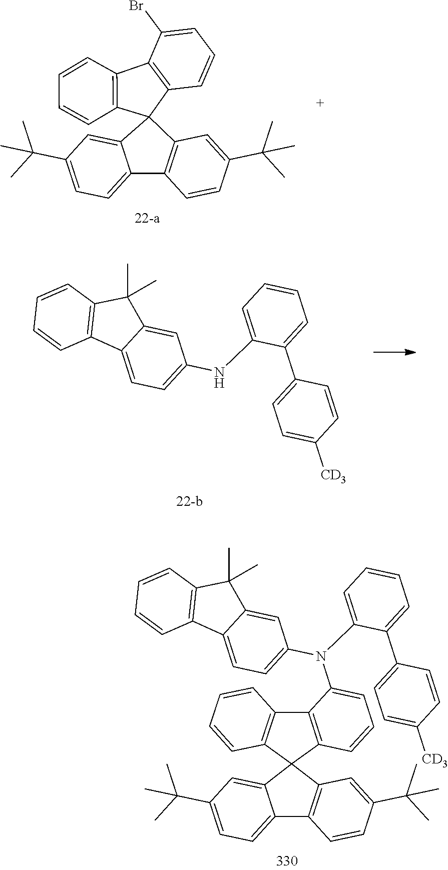 [SMILES: CC(C)(C)c1ccc2c(c1)C1(c3cc(C(C)(C)C)ccc3-2)c2ccccc2-c2c(Br)cccc21 ; [2H]C([2H])([2H])c1ccc(-c2ccccc2Nc2ccc3c(c2)C(C)(C)c2ccccc2-3)cc1 ; [2H]C([2H])([2H])c1ccc(-c2ccccc2N(c2ccc3c(c2)C(C)(C)c2ccccc2-3)c2cccc3c2-c2ccccc2C32c3cc(C(C)(C)C)ccc3-c3ccc(C(C)(C)C)cc32)cc1]

Under the protection of nitrogen, compound 20-a (4 g, 507.50 g/mol, 7.88 mmol), compound 20-b (1 eq, 2.98 g, 378.52 g/mol, 7.88 mmol), sodium tert-butoxide (1.1 eq, 0.83 g, 96.1 g/mol, 8.67 mmol), tris(dibenzylideneacetone) dipalladium (0.05 eq, 0.36 g, 915 g/mol, 0.39 mmol), tri-tert-butylphosphine (0.05 eq, 0.079 g, 202.32 g/mol, 0.39 mmol) and toluene (40 ml) were added into a reaction flask, heated for reflux reaction for 5 h after the materials were added, cooled to room temperature after the reaction was finished, added with water (40 ml), stirred for 15 min, and filtered to obtain a filtrate, and the filtrate was filtered by diatomite and then separated to obtain an organic phase, and the organic phase was dried by anhydrous magnesium sulfate and then spin-dried, and purified by column chromatography to obtain the novel organic electroluminescent compound 330 (3.85 g, yield 60.7%), ESI-MS (m/z) (M+): theoretical value 805.11, observed value 805.32.

All of the intermediate compounds 1-a, 2-a, 3-a, 4-a, 5-a, 6-a, 7-a, 8-a, 9-a, 10-a, 11-a, 12-a, 13-a, 14-a, 15-a, 16-a, 17-a, 18-a, 19-a, 20-a, 21-a, 22-a, sodium tert-butoxide, tris(dibenzylideneacetone) dipalladium, tri-tert-butylphosphine, toluene and anhydrous magnesium sulfate in Examples 1-20 can be purchased or customized from domestic chemical product market, for example, available from Yurui (Shanghai) Chemical Co., Ltd, Sinopharm Chemical Reagent Co., Ltd and J&K Scientific Ltd. Besides, they can be synthesized by those skilled in the art by commonly known methods.

The compounds 1-b, 2-b, 3-b, 4-b, 5-b, 6-b, 7-b, 8-b, 9-b, 10-b, 11-b, 12-b, 13-b, 14-b, 15-b, 16-b, 17-b, 18-b, 19-b, 20-b, 21-b and 22-b are synthesized by the following method, and both raw material 1 and raw material 2 used in the synthesis can be purchased or customized from domestic chemical product market, for example, available from Yurui (Shanghai) Chemical Co., Ltd, Sinopharm Chemical Reagent Co., Ltd and J&K Scientific Ltd., and they also can be synthesized by those skilled in the art by commonly known methods.

| | | | Synthetic |
|---|---|---|---|
| Raw material 1 | Raw material 2 | Target product | method: |
| 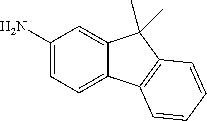 [SMILES: CC1(C)c2ccccc2-c2ccc(N)cc21] | 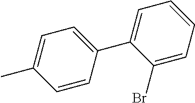 | 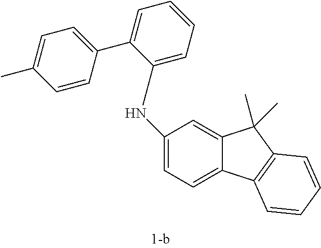 [SMILES: Cc1ccc(-c2ccccc2Nc2ccc3c(c2)C(C)(C)c2ccccc2-3)cc1] | Under the protection of nitrogen, the raw material 1, the raw material 2, sodium tert- butoxide, tris(dibenzyl- ideneacetone) dipalladium, tri-tert-butyl- phosphine and toluene were added |
| | | | into a reaction |
| | | | flask, heated |
| | | | for reflux |
| | | | reaction, then |
| | | | cooled to room |
| | | | temperature, |
| | | | added with |
| | | | water, stirred, |
| | | | and filtered to |
| | | | obtain a filtrate, |
| | | | the filtrate was |
| | | | separated to |
| | | | obtain an |
| | | | organic phase, |
| | | | and the organic |
| | | | phase was dried |
| | | | by anhydrous |
| | | | magnesium |
| | | | sulfate and |
| | | | then spin- |
| | | | dried, and |
| | | | purified by |
| | | | column chrom- |
| | | | atography to |
| | | | obtain 1-b |
| | | | (yield 66.5%), |
| | | | ESI-MS (m/z) |
| | | | (M+): theoret- |
| | | | ical value |
| | | | 375.50, |
| | | | observed |
| | | | value 375.63. |
| 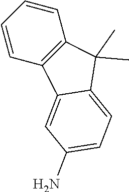 | 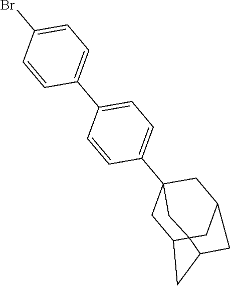 [SMILES: Brc1ccc(-c2ccc(C34CC5CC6CC(C3)C6(C5)C4)cc2)cc1] | 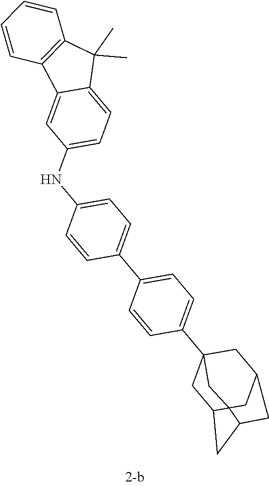 | Refer to the synthetic method of compound 1-b (yield 65.9%), ESI-MS (m/z) (M+): theoretical value 495.70, and observed value 495.44. |
| 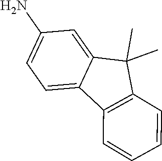 | 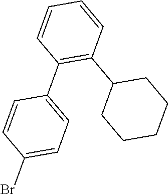 [SMILES: Brc1ccc(-c2ccccc2C2CCCCC2)cc1] | 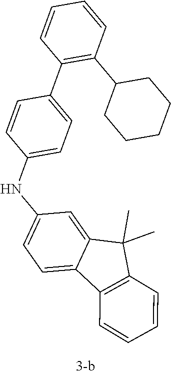 [SMILES: CC1(C)c2ccccc2-c2ccc(Nc3ccc(-c4ccccc4C4CCCCC4)cc3)cc21] | Refer to the synthetic method of compound 1-b (yield 75.2%), ESI-MS (m/z) (M+): theoretical value 443.62, and observed value 443.76. |
| 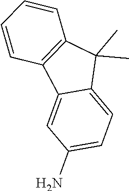 | 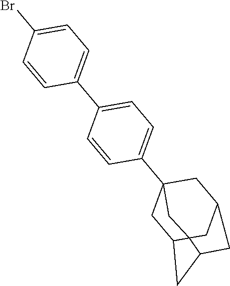 [SMILES: Brc1ccc(-c2ccc(C34CC5CC6CC(C3)C6(C5)C4)cc2)cc1] | 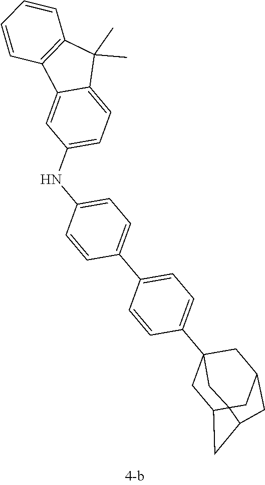 | Refer to the synthetic method of compound 1-b (yield 65.9%), ESI-MS (m/z) (M+): theoretical value 495.70, and observed value 495.44. |
| 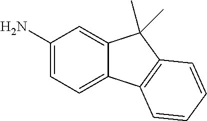 | 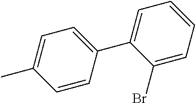 | 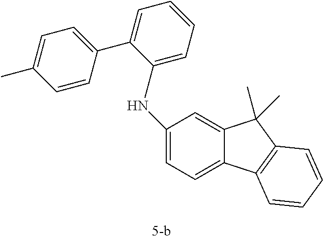 [SMILES: Cc1ccc(-c2ccccc2Nc2ccc3c(c2)C(C)(C)c2ccccc2-3)cc1] | Refer to the synthetic method of compound 1-b (yield 66.5%), ESI-MS (m/z) (M+): theoretical value 375.50, and observed value 375.63. |
| 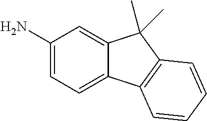 | 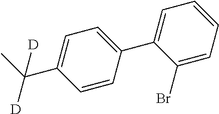 | 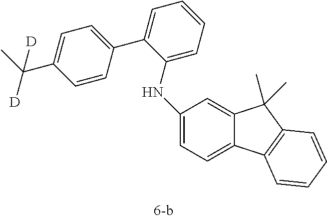 [SMILES: [2H]C([2H])(C)c1ccc(-c2ccccc2Nc2ccc3c(c2)C(C)(C)c2ccccc2-3)cc1] | Refer to the synthetic method of compound 1-b (yield 71.2%), ESI-MS (m/z) (M+): theoretical value 391.54, and observed value 391.69. |
| 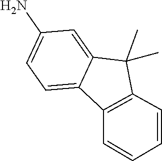 | 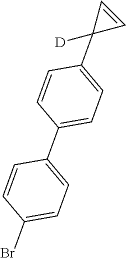 [SMILES: [2H]C1(c2ccc(-c3ccc(Br)cc3)cc2)C=C1] | 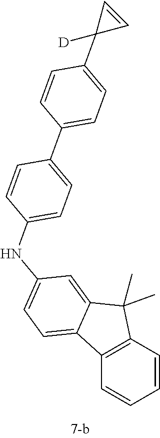 [SMILES: [2H]C1(c2ccc(-c3ccc(Nc4ccc5c(c4)C(C)(C)c4ccccc4-5)cc3)cc2)C=C1] | Refer to the synthetic method of compound 1-b (yield 68.4%), ESI-MS (m/z) (M+): theoretical value 400.53, and observed value 401.15. |
| 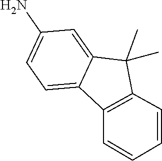 | 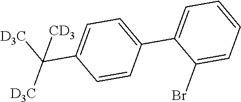 [SMILES: [2H]C([2H])([2H])C(c1ccc(-c2ccccc2Br)cc1)(C([2H])([2H])[2H])C([2H])([2H])[2H]] | 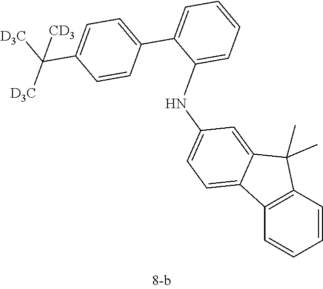 [SMILES: [2H]C([2H])([2H])C(c1ccc(-c2ccccc2Nc2ccc3c(c2)C(C)(C)c2ccccc2-3)cc1)(C([2H])([2H])[2H])C([2H])([2H])[2H]] | Refer to the synthetic method of compound 1-b (yield 66.3%), ESI-MS (m/z) (M+): theoretical value 426.64, and observed value 427.05. |
| 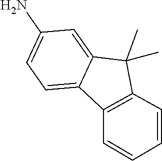 | 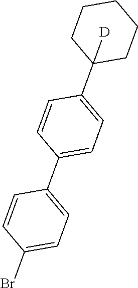 | 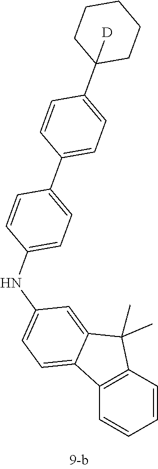 | Refer to the synthetic method of compound 1-b (yield 64.6%), ESI-MS (m/z) (M+): theoretical value 444.63, and observed value 445.21. |
| 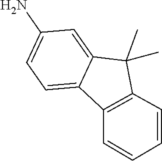 | 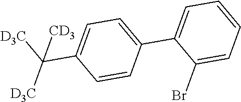 [SMILES: [2H]C([2H])([2H])C(c1ccc(-c2ccccc2Br)cc1)(C([2H])([2H])[2H])C([2H])([2H])[2H]] | 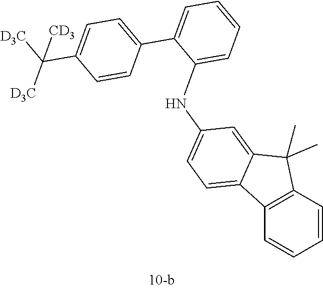 [SMILES: [2H]C([2H])([2H])C(c1ccc(-c2ccccc2Nc2ccc3c(c2)C(C)(C)c2ccccc2-3)cc1)(C([2H])([2H])[2H])C([2H])([2H])[2H]] | Refer to the synthetic method of compound 1-b (yield 66.3%), ESI-MS (m/z) (M+): theoretical value 426.64, and observed value 427.05. |
| 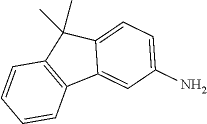 | 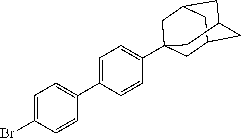 [SMILES: Brc1ccc(-c2ccc(C34CC5CC(CC(C5)C3)C4)cc2)cc1] | 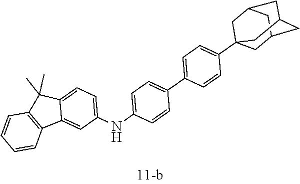 [SMILES: CC1(C)c2ccccc2-c2cc(Nc3ccc(-c4ccc(C56CC7CC(CC(C7)C5)C6)cc4)cc3)ccc21] | Refer to the synthetic method of compound 1-b (yield 65.9%), ESI-MS (m/z) (M+): theoretical value 495.70, and observed value 495.44. |
| 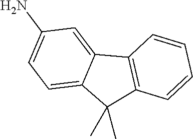 | 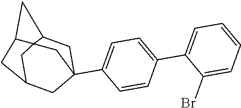 [SMILES: Brc1ccccc1-c1ccc(C23CC4CC5CC(C2)C5(C4)C3)cc1] | 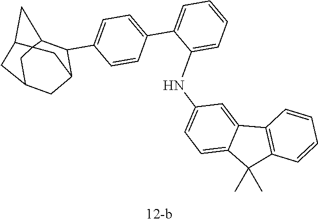 | Refer to the synthetic method of compound 1-b (yield 61.5%), ESI-MS (m/z) (M+): theoretical value 495.70, and observed value 495.97. |
| 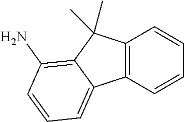 | 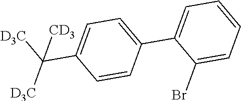 [SMILES: [2H]C([2H])([2H])C(c1ccc(-c2ccccc2Br)cc1)(C([2H])([2H])[2H])C([2H])([2H])[2H]] | 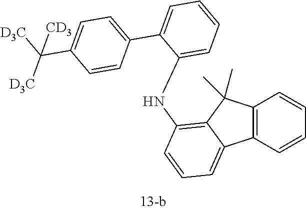 [SMILES: [2H]C([2H])([2H])C(c1ccc(-c2ccccc2Nc2cccc3c2C(C)(C)c2ccccc2-3)cc1)(C([2H])([2H])[2H])C([2H])([2H])[2H]] | Refer to the synthetic method of compound 1-b (yield 58.8%), ESI-MS (m/z) (M+): theoretical value 426.64, and observed value 427.10. |
| 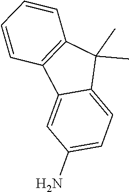 | 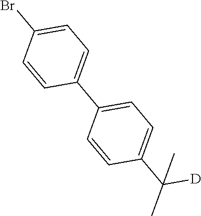 | 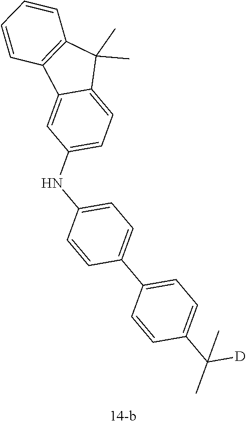 [SMILES: [2H]C(C)(C)c1ccc(-c2ccc(Nc3ccc4c(c3)-c3ccccc3C4(C)C)cc2)cc1] | Refer to the synthetic method of compound 1-b (yield 70.2%), ESI-MS (m/z) (M+): theoretical value 404.56, and observed value 403.98. |
| 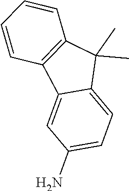 | 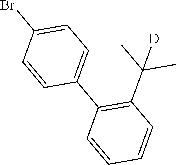 | 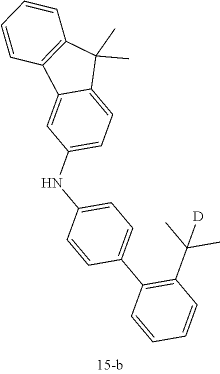 [SMILES: [2H]C(C)(C)c1ccccc1-c1ccc(Nc2ccc3c(c2)-c2ccccc2C3(C)C)cc1] | Refer to the synthetic method of compound 1-b (yield 63.3%), ESI-MS (m/z) (M+): theoretical value 404.56, and observed value 403.90. |
| 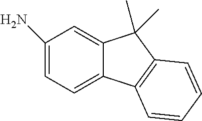 | 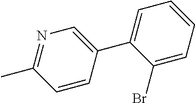 | 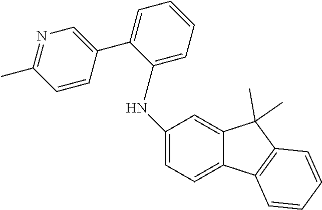 [SMILES: Cc1ccc(-c2ccccc2Nc2ccc3c(c2)C(C)(C)c2ccccc2-3)cn1] | Refer to the synthetic method of compound 1-b (yield 64.7%), ESI-MS (m/z) (M+): theoretical value 376.49, and observed value 376.72. |
| 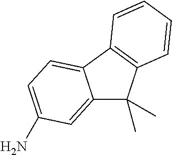 | 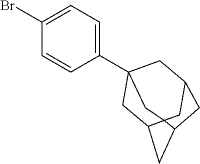 [SMILES: Brc1ccc(C23CC4CC5CC(C2)C5(C4)C3)cc1] | 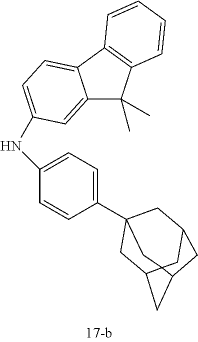 | Refer to the synthetic method of compound 1-b (yield 72.5%), ESI-MS (m/z) (M+): theoretical value 419.60, and observed value 419.79. |
| 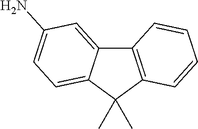 | 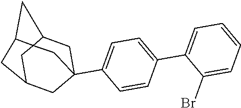 [SMILES: Brc1ccccc1-c1ccc(C23CC4CC5CC(C2)C5(C4)C3)cc1] | 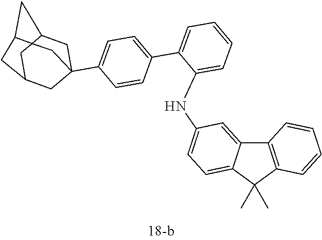 | Refer to the synthetic method of compound 1-b (yield 61.5%), ESI-MS (m/z) (M+): theoretical value 495.70, and observed value 495.97. |
| 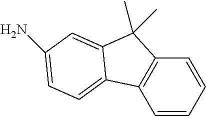 | 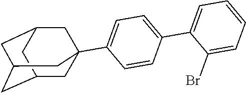 [SMILES: Brc1ccccc1-c1ccc(C23CC4CC(CC(C4)C2)C3)cc1] | 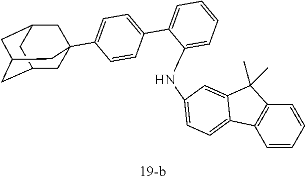 [SMILES: CC1(C)c2ccccc2-c2ccc(Nc3ccccc3-c3ccc(C45CC6CC(CC(C6)C4)C5)cc3)cc21] | Refer to the synthetic method of compound 1-b (yield 63.9%), ESI-MS (m/z) (M+): theoretical value 495.70, and observed value 495.94. |
| 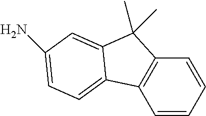 | 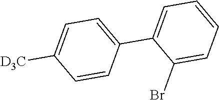 [SMILES: [2H]C([2H])([2H])c1ccc(-c2ccccc2Br)cc1] | 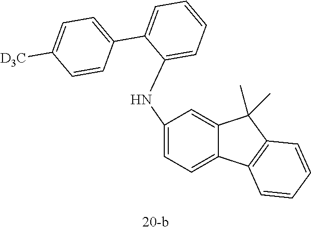 [SMILES: [2H]C([2H])([2H])c1ccc(-c2ccccc2Nc2ccc3c(c2)C(C)(C)c2ccccc2-3)cc1] | Refer to the synthetic method of compound 1-b (yield 65.5%), ESI-MS (m/z) (M+): theoretical value 378.52, and observed value 279.03. |
| 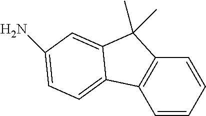 | 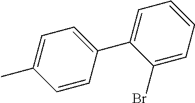 | 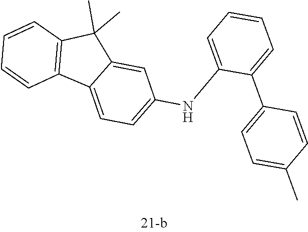 [SMILES: Cc1ccc(-c2ccccc2Nc2ccc3c(c2)C(C)(C)c2ccccc2-3)cc1] | Refer to the synthetic method of compound 1-b (yield 66.5%), ESI-MS (m/z) (M+): theoretical value 375.50, and observed value 375.63. |
| 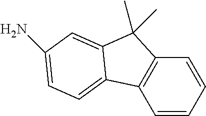 | 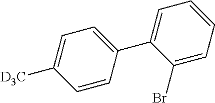 [SMILES: [2H]C([2H])([2H])c1ccc(-c2ccccc2Br)cc1] | 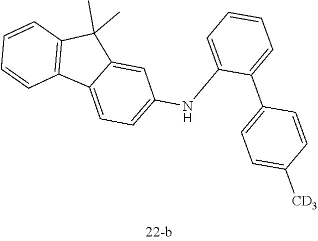 [SMILES: [2H]C([2H])([2H])c1ccc(-c2ccccc2Nc2ccc3c(c2)C(C)(C)c2ccccc2-3)cc1] | Refer to the synthetic method of compound 1-b (yield 65.5%), ESI-MS (m/z) (M+): theoretical value 378.52, and observed value 279.03. |

Testing the Material Properties:

HT-1 and the novel organic electroluminescent compounds 5, 48, 62, 64, 73, 97, 131, 157, 183, 208, 211, 270, 278, 298, 305, 325, 43, 204, 152, 145, 329 and 330 of the present disclosure were tested for the thermogravimetric temperature Td, and test results are shown in Table 1 below.

Note: the thermogravimetric temperature Td is the temperature at which the weight loss is 5% in a nitrogen atmosphere, and is measured on a TGA N-1000 thermogravimetric analyzer, and the nitrogen flow is 10 mL/min during the test.

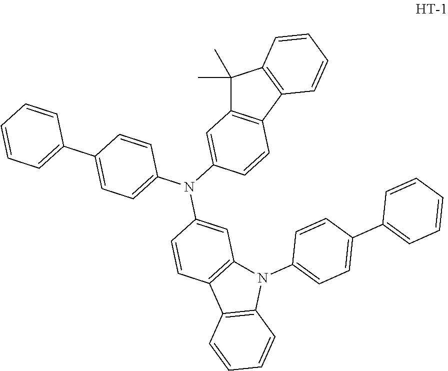 [SMILES: CC1(C)c2ccccc2-c2ccc(N(c3ccc(-c4ccccc4)cc3)c3ccc4c5ccccc5n(-c5ccc(-c6ccccc6)cc5)c4c3)cc21]

TABLE 1

| Item | Material | Td/° C. |
|---|---|---|
| Comparative | HT-1 | 398.58 |
| Example | | |
| Example 1 | 5 | 417.58 |
| Example 2 | 48 | 419.52 |
| Example 3 | 62 | 428.20 |
| Example 4 | 64 | 430.12 |
| Example 5 | 73 | 422.54 |
| Example 6 | 97 | 427.53 |
| Example 7 | 131 | 431.25 |
| Example 8 | 157 | 428.80 |
| Example 9 | 183 | 425.19 |
| Example 10 | 208 | 432.06 |
| Example 11 | 211 | 415.04 |
| Example 12 | 270 | 426.43 |
| Example 13 | 278 | 427.42 |
| Example 14 | 298 | 436.25 |
| Example 15 | 305 | 416.74 |
| Example 16 | 325 | 433.51 |
| Example 17 | 43 | 420.43 |
| Example 18 | 204 | 427.13 |
| Example 19 | 152 | 432.57 |
| Example 20 | 145 | 427.51 |
| Example 21 | 329 | 435.28 |
| Example 22 | 330 | 429.47 |

It can be seen from the above data that the thermal stability of the novel organic electroluminescent compounds of the present disclosure is better than that of the comparative example HT-1, which indicates that all the novel organic electroluminescent compounds according to the general structural formula of the present disclosure have excellent thermal stability, and can meet the requirements of use of organic electroluminescent materials.

Testing the performance of the device:

### Application Example 1

adopting ITO as a reflecting layer anode substrate material, and sequentially carrying out surface treatment on the ITO using water, acetone, and N₂ plasma;

depositing HAT-CN with a thickness of 10 nm to form a hole injection layer (HIL) on the ITO anode substrate;

vapor depositing, by evaporation, the novel organic electroluminescent compound 5 prepared in Example 1 of the present disclosure on the hole injection layer (HIL) to form a hole transport layer (HTL) with a thickness of 120 nm;

vapor depositing, by evaporation, ADN as a blue light main material and BD-1 as a blue light doping material (a use amount of BD-1 is 5% of the weight of ADN) at different rates to form a light-emitting layer with a thickness of 30 nm on the hole transport layer (HTL);

vapor depositing, by evaporation, PBD on the light-emitting layer to obtain an electron transport layer (ETL) with a thickness of 35 nm, and vapor depositing, by evaporation, LiQ with a thickness of 2 nm on the electron transport layer (ETL) to form an electron injection layer (EIL); and

subsequently, mixing magnesium (Mg) and silver (Ag) at a ratio of 9:1 to obtain a mixture and vapor depositing, by evaporation, the mixture to obtain a cathode with a thickness of 15 nm, depositing DNTPD with a thickness of 50 nm on the above-mentioned cathode sealing layer, and further, sealing the surface of the cathode with a UV curable adhesive and a sealing film (seal cap) containing a moisture scavenger so as to protect the organic electroluminescent device from oxygen or moisture in atmosphere, thus obtaining an organic electroluminescent device.

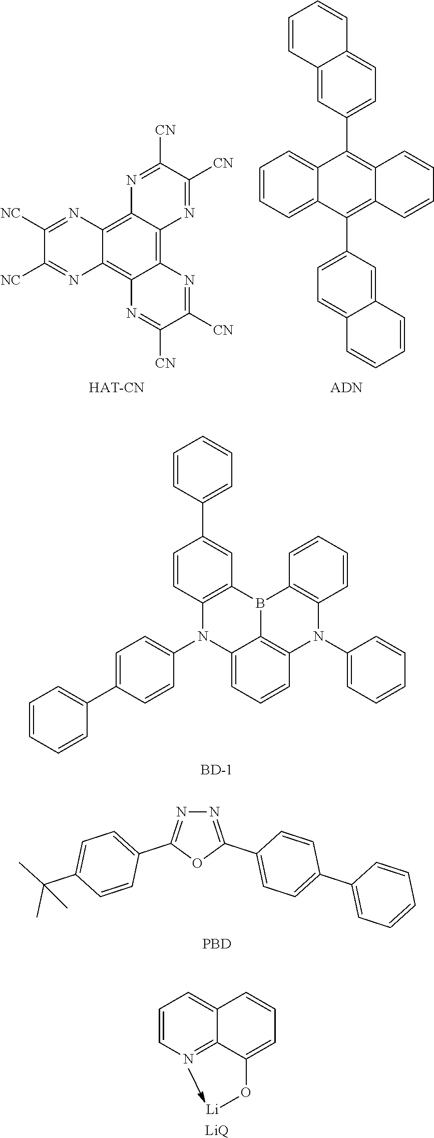

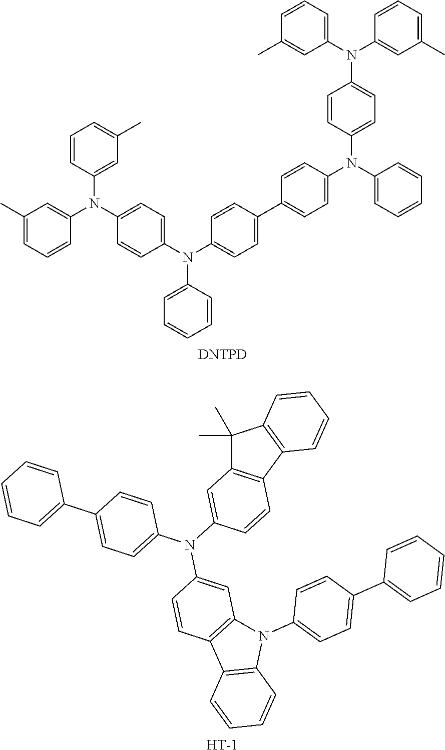

### Application Example 2-22

Organic electroluminescent devices of Application Examples 2-19 were produced using the novel organic electroluminescent compounds 48, 62, 64, 73, 97, 131, 157, 183, 208, 211, 270, 278, 298, 305, 325, 43, 204, 152, 145, 329 and 330 in Examples 2-22 of the present disclosure as hole transport layer (HTL) material, respectively, and the other aspects being identical to those of Application Example 1.

### Comparative Example

The Comparative Example is different from Application Example 1 in that HT-1 was used as a hole transport layer (HTL) material, and the rest was the same as Application Example 1.

The characteristics of the organic electroluminescent devices produced in the above application examples and the organic electroluminescent device produced in the comparative example were measured under the condition that the current density was 10 mA/cm², and results are shown in Table 2.

TABLE 2

| | Hole | | | | |
|---|---|---|---|---|---|
| | Transport | | | Light- | |
| | Layer | Current | | emitting | |
| Experiment | (HTL) | Density | Voltage | Efficiency | CIE |
| Group | Material | (mA/cm²) | (V) | (Cd/A) | (x, y) |
| Comparative | HT-1 | 10 | 4.54 | 10.2 | (0.1220, |
| Example | | | | | 0.1003) |
| Application | 5 | 10 | 4.02 | 12.4 | (0.1201, |
| Example 1 | | | | | 0.1102) |
| Application | 48 | 10 | 3.96 | 13.5 | (0.1182, |
| Example 2 | | | | | 0.1106) |
| Application | 62 | 10 | 3.94 | 14.1 | (0.1206, |
| Example 3 | | | | | 0.1065) |
| Application | 64 | 10 | 4.05 | 13.2 | (0.1196, |
| Example 4 | | | | | 0.1083) |
| Application | 73 | 10 | 3.95 | 13.8 | (0.1190, |
| Example 5 | | | | | 0.1105) |
| Application | 97 | 10 | 3.88 | 14.0 | (0.1188, |
| Example 6 | | | | | 0.1100) |
| Application | 131 | 10 | 3.94 | 12.8 | (0.1120, |
| Example 7 | | | | | 0.1045) |
| Application | 157 | 10 | 4.03 | 12.3 | (0.1210, |
| Example 8 | | | | | 0.1042) |
| Application | 183 | 10 | 4.10 | 11.9 | (0.1180, |
| Example 9 | | | | | 0.1123) |
| Application | 208 | 10 | 3.97 | 12.3 | (0.1202, |
| Example 10 | | | | | 0.1083) |
| Application | 211 | 10 | 4.03 | 13.8 | (0.1195, |
| Example 11 | | | | | 0.1108) |
| Application | 270 | 10 | 3.94 | 12.4 | (0.1210, |
| Example 12 | | | | | 0.1094) |
| Application | 278 | 10 | 3.99 | 13.3 | (0.1184, |
| Example 13 | | | | | 0.1073) |
| Application | 298 | 10 | 3.95 | 12.7 | (0.1190, |
| Example 14 | | | | | 0.1044) |
| Application | 305 | 10 | 3.94 | 12.8 | (0.1203, |
| Example 15 | | | | | 0.1100) |
| Application | 325 | 10 | 4.04 | 12.1 | (0.1217, |
| Example 16 | | | | | 0.1078) |
| Application | 43 | 10 | 4.09 | 13.0 | (0.1202, |
| Example 17 | | | | | 0.1092) |
| Application | 204 | 10 | 3.96 | 12.6 | (0.1186, |
| Example 18 | | | | | 0.1065) |
| Application | 152 | 10 | 3.86 | 11.9 | (0.1176, |
| Example 19 | | | | | 0.1084) |
| Application | 145 | 10 | 4.09 | 10.8 | (0.1182, |
| Example 20 | | | | | 0.1065) |
| Application | 329 | 10 | 3.92 | 12.2 | (0.1198, |
| Example 21 | | | | | 0.1034) |
| Application | 330 | 10 | 4.05 | 13.1 | (0.1205, |
| Example 22 | | | | | 0.1050) |

As can be seen from Table 2 above, when the novel organic electroluminescent compound of the present disclosure is applied to an organic electroluminescent device, the light-emitting efficiency is greatly improved under the same current density, the starting voltage of the device is reduced to some extent, the power consumption of the device is relatively reduced, and the service life of the device is correspondingly improved.

The organic electroluminescent devices prepared in the comparative example, Application Example 1, Application Example 2, Application Example 5 and Application Example 13 were subjected to a test for light-emitting service life to obtain the light-emitting service life T97% data (time for which the light-emitting brightness was decreased to 97% of initial brightness), and test apparatus was a TEO light-emitting device service life test system. Results are shown in Table 3:

TABLE 3

| | Current Density | |
|---|---|---|
| Experiment Group | (mA/cm²) | T97%/h |
| Comparative Example | 10 | 251 |
| Application Example 1 | 10 | 274 |
| Application Example 2 | 10 | 280 |
| Application Example 5 | 10 | 271 |
| Application Example 13 | 10 | 289 |

As can be seen from above Table 3, when the novel organic electroluminescent compound of the present disclosure is applied to an organic electroluminescent device, the service life is greatly prolonged under the same current density, with a wide application prospect.

1-20. (canceled)

21. A novel organic electroluminescent compound, having a structural formula represented as follows:

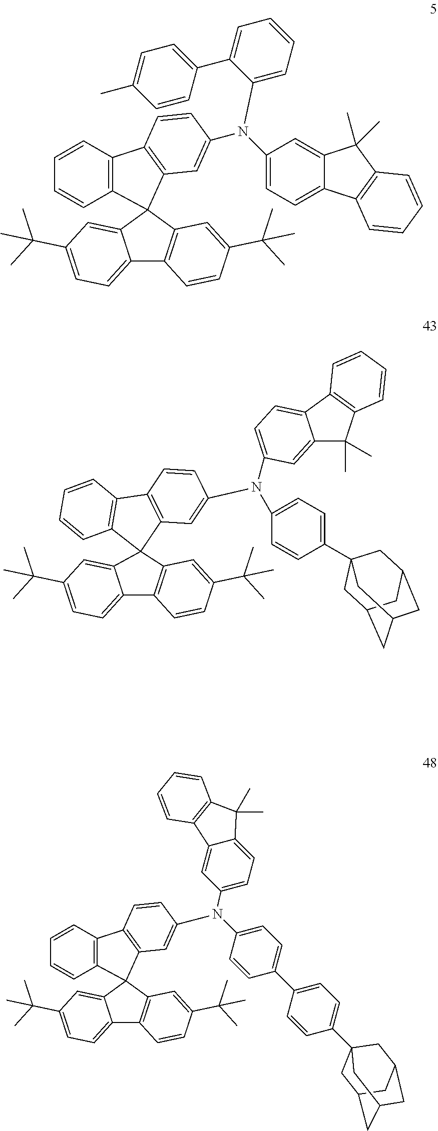 [SMILES: Cc1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1 ; CC(C)(C)c1ccc2c(c1)C1(c3ccccc3-c3ccc(N(c4ccc(C56CC7CC(CC(C7)C5)C6)cc4)c4ccc5c(c4)C(C)(C)c4ccccc4-5)cc31)c1cc(C(C)(C)C)ccc1-2 ; CC(C)(C)c1ccc2c(c1)C1(c3ccccc3-c3ccc(N(c4ccc(-c5ccc(C67CC8CC(CC(C8)C6)C7)cc5)cc4)c4ccc5c(c4)-c4ccccc4C5(C)C)cc31)c1cc(C(C)(C)C)ccc1-2]

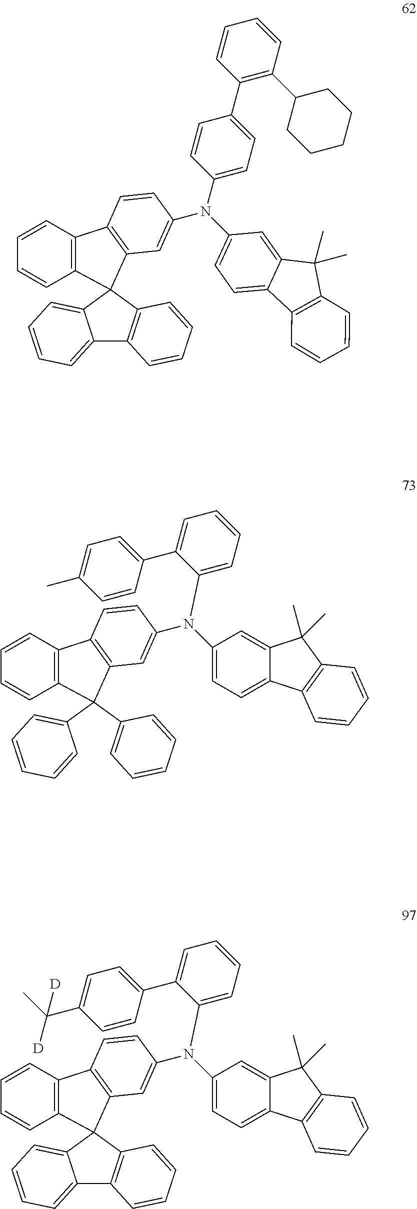 [SMILES: CC1(C)c2ccccc2-c2ccc(N(c3ccc(-c4ccccc4C4CCCCC4)cc3)c3ccc4c(c3)C3(c5ccccc5-c5ccccc53)c3ccccc3-4)cc21 ; Cc1ccc(-c2ccccc2N(c2ccc3c(c2)C(C)(C)c2ccccc2-3)c2ccc3c(c2)C(c2ccccc2)(c2ccccc2)c2ccccc2-3)cc1 ; [2H]C([2H])(C)c1ccc(-c2ccccc2N(c2ccc3c(c2)C(C)(C)c2ccccc2-3)c2ccc3c(c2)C2(c4ccccc4-c4ccccc42)c2ccccc2-3)cc1]

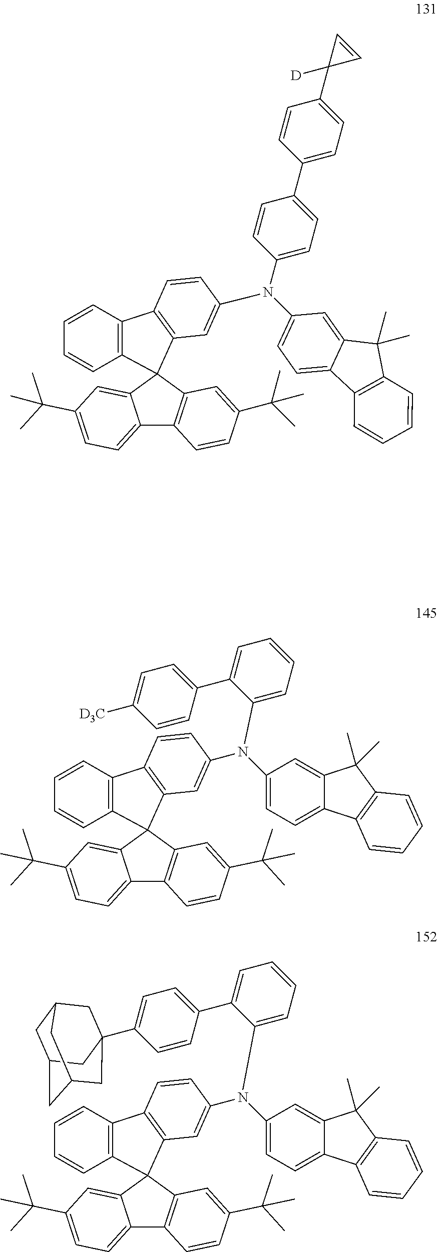 [SMILES: [2H]C1(c2ccc(-c3ccc(N(c4ccc5c(c4)C(C)(C)c4ccccc4-5)c4ccc5c(c4)C4(c6ccccc6-5)c5cc(C(C)(C)C)ccc5-c5ccc(C(C)(C)C)cc54)cc3)cc2)C=C1 ; [2H]C([2H])([2H])c1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1 ; CC(C)(C)c1ccc2c(c1)C1(c3ccccc3-c3ccc(N(c4ccc5c(c4)C(C)(C)c4ccccc4-5)c4ccccc4-c4ccc(C56CC7CC(CC(C7)C5)C6)cc4)cc31)c1cc(C(C)(C)C)ccc1-2]

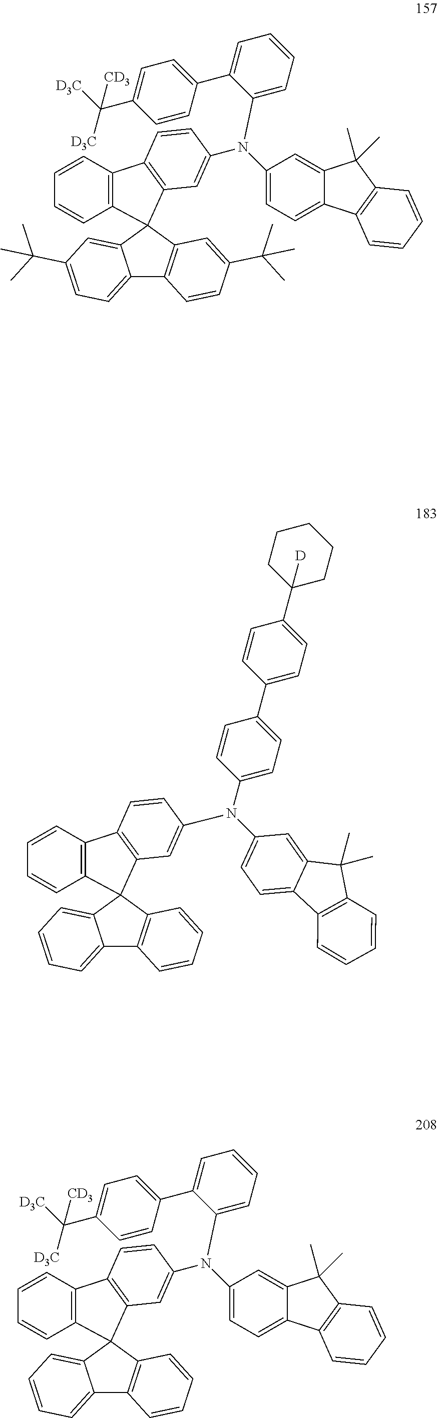

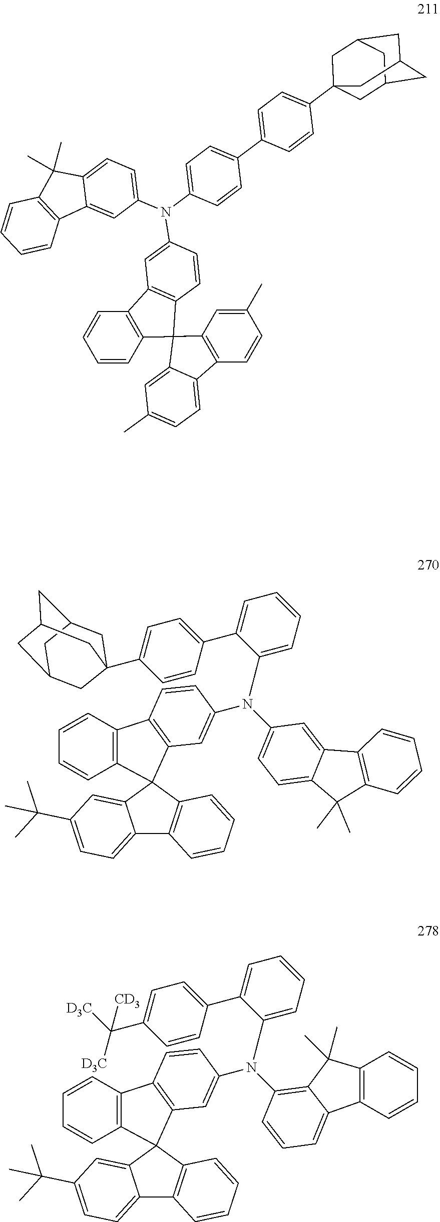 [SMILES: Cc1ccc2c(c1)C1(c3ccccc3-c3cc(N(c4ccc(-c5ccc(C67CC8CC(CC(C8)C6)C7)cc5)cc4)c4ccc5c(c4)-c4ccccc4C5(C)C)ccc31)c1cc(C)ccc1-2 ; CC(C)(C)c1ccc2c(c1)C1(c3ccccc3-c3ccc(N(c4ccc5c(c4)-c4ccccc4C5(C)C)c4ccccc4-c4ccc(C56CC7CC(CC(C7)C5)C6)cc4)cc31)c1ccccc1-2 ; [2H]C([2H])([2H])C(c1ccc(-c2ccccc2N(c2ccc3c(c2)C2(c4ccccc4-3)c3ccccc3-c3ccc(C(C)(C)C)cc32)c2cccc3c2C(C)(C)c2ccccc2-3)cc1)(C([2H])([2H])[2H])C([2H])([2H])[2H]]

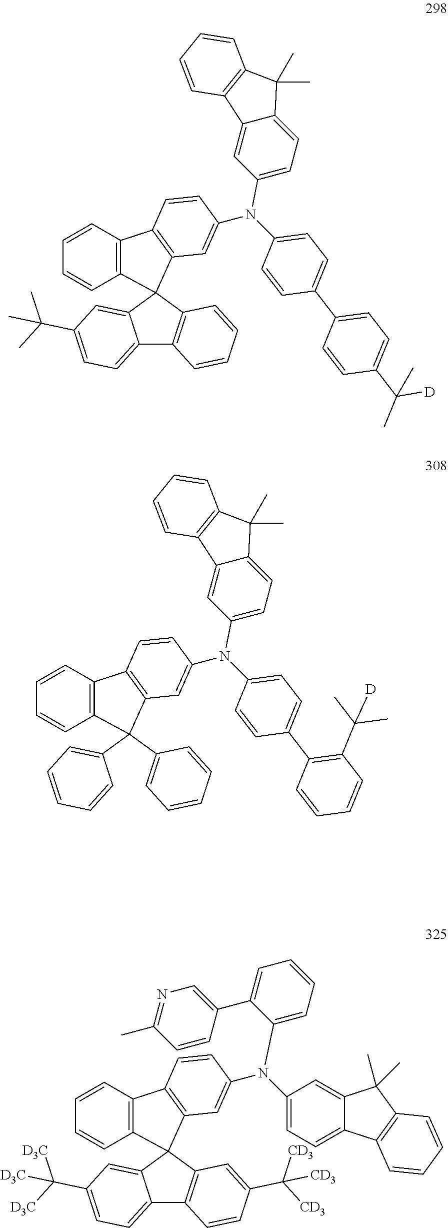 [SMILES: [2H]C(C)(C)c1ccc(-c2ccc(N(c3ccc4c(c3)-c3ccccc3C4(C)C)c3ccc4c(c3)C3(c5ccccc5-4)c4ccccc4-c4ccc(C(C)(C)C)cc43)cc2)cc1 ; [2H]C(C)(C)c1ccccc1-c1ccc(N(c2ccc3c(c2)-c2ccccc2C3(C)C)c2ccc3c(c2)C(c2ccccc2)(c2ccccc2)c2ccccc2-3)cc1 ; [2H]C([2H])([2H])C(c1ccc2c(c1)C1(c3ccccc3-c3ccc(N(c4ccc5c(c4)C(C)(C)c4ccccc4-5)c4ccccc4-c4ccc(C)nc4)cc31)c1cc(C(C([2H])([2H])[2H])(C([2H])([2H])[2H])C([2H])([2H])[2H])ccc1-2)(C([2H])([2H])[2H])C([2H])([2H])[2H]]

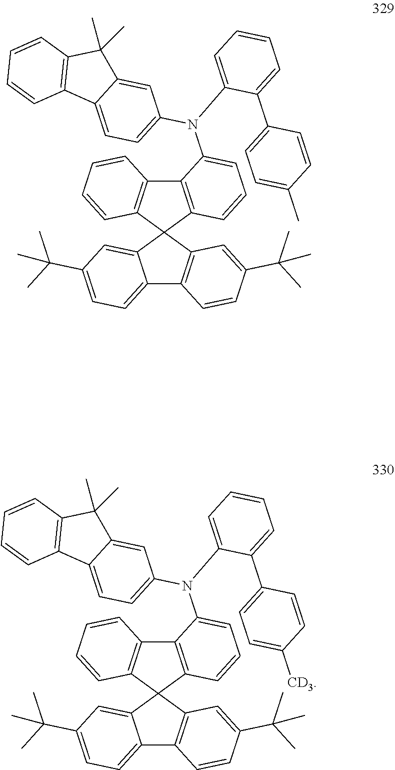 [SMILES: Cc1ccc(-c2ccccc2N(c2ccc3c(c2)C(C)(C)c2ccccc2-3)c2cccc3c2-c2ccccc2C32c3cc(C(C)(C)C)ccc3-c3ccc(C(C)(C)C)cc32)cc1 ; [2H]C([2H])([2H])c1ccc(-c2ccccc2N(c2ccc3c(c2)C(C)(C)c2ccccc2-3)c2cccc3c2-c2ccccc2C32c3cc(C(C)(C)C)ccc3-c3ccc(C(C)(C)C)cc32)cc1]

22. The novel organic electroluminescent compound according to claim 21, having a structural formula represented as follows:

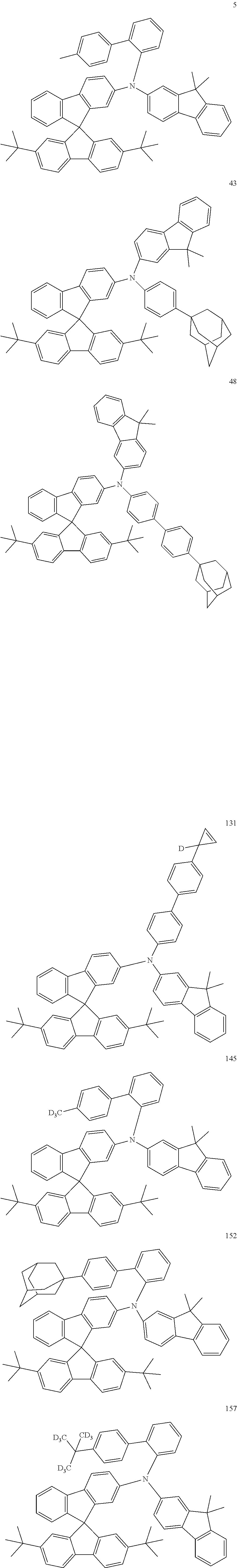

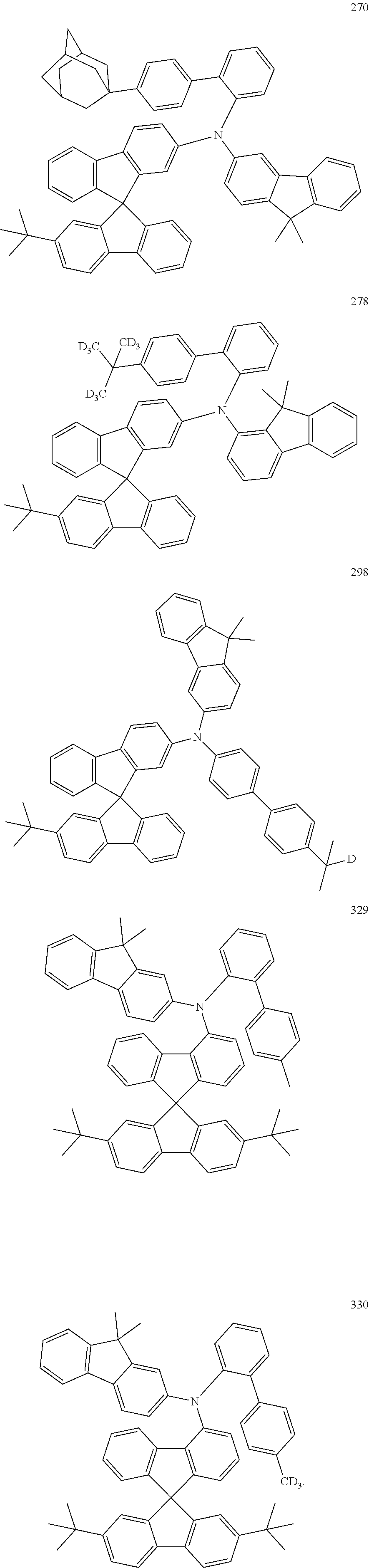

23. The novel organic electroluminescent compound according to claim 21, having a structural formula represented as follows:

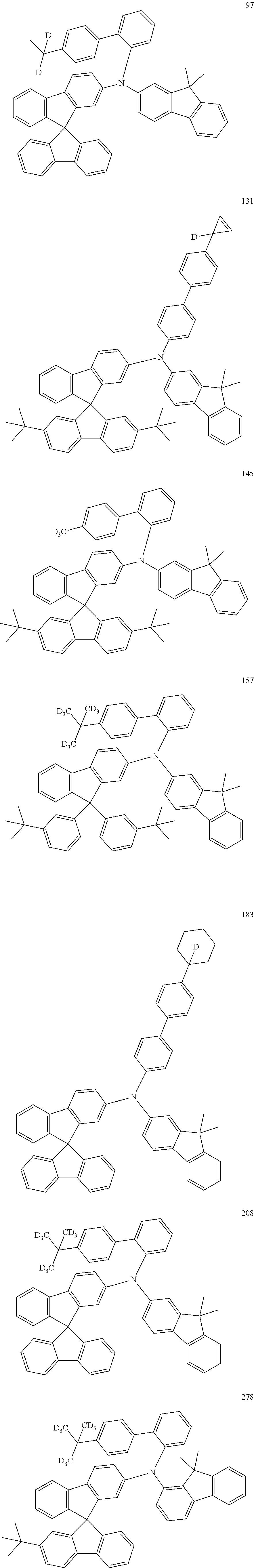

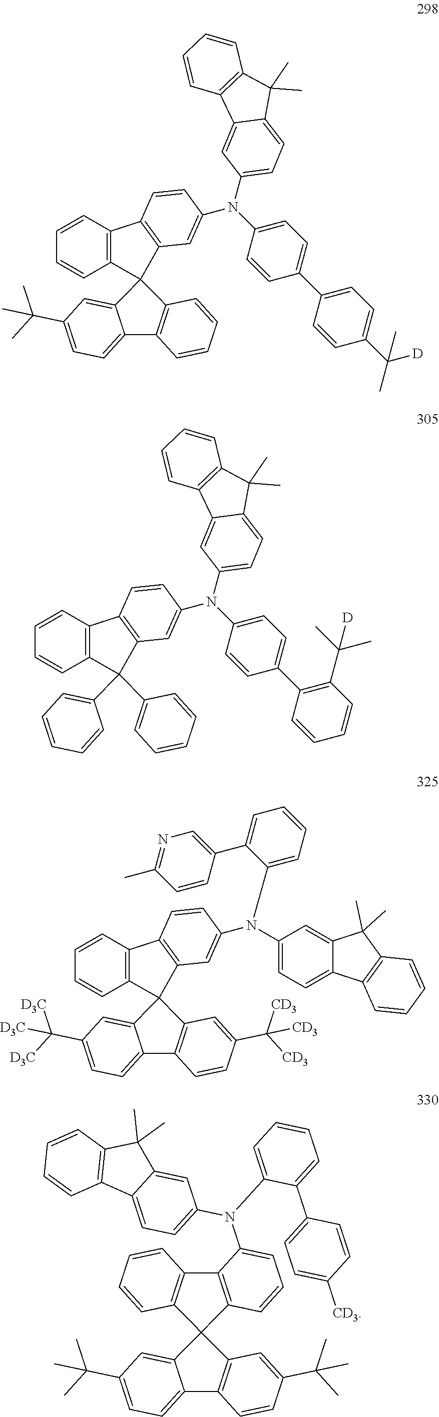

24. The novel organic electroluminescent compound according to claim 21, having a structural formula represented as follows:

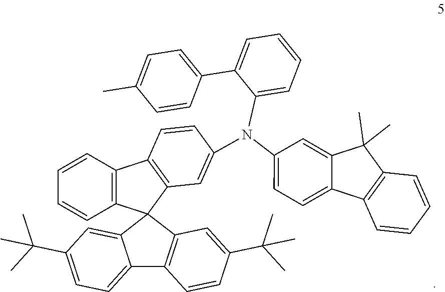 [SMILES: Cc1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1]

25. The novel organic electroluminescent compound according to claim 21, having a structural formula represented as follows:

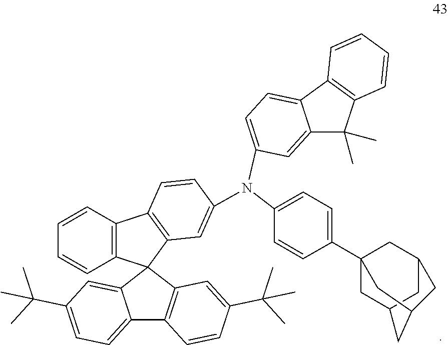 [SMILES: CC(C)(C)c1ccc2c(c1)C1(c3ccccc3-c3ccc(N(c4ccc(C56CC7CC(CC(C7)C5)C6)cc4)c4ccc5c(c4)C(C)(C)c4ccccc4-5)cc31)c1cc(C(C)(C)C)ccc1-2]

26. The novel organic electroluminescent compound according to claim 21, having a structural formula represented as follows:

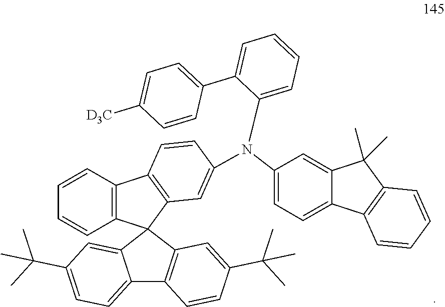 [SMILES: [2H]C([2H])([2H])c1ccc(-c2ccccc2N(c2ccc3c(c2)C(C)(C)c2ccccc2-3)c2ccc3c(c2)C2(c4ccccc4-3)c3cc(C(C)(C)C)ccc3-c3ccc(C(C)(C)C)cc32)cc1]

27. The novel organic electroluminescent compound according to claim 21, having a structural formula represented as follows:

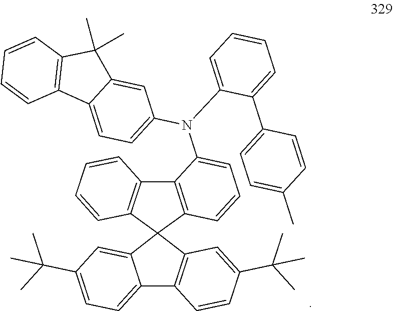 [SMILES: Cc1ccc(-c2ccccc2N(c2ccc3c(c2)C(C)(C)c2ccccc2-3)c2cccc3c2-c2ccccc2C32c3cc(C(C)(C)C)ccc3-c3ccc(C(C)(C)C)cc32)cc1]

28. The novel organic electroluminescent compound according to claim 21, having a structural formula represented as follows:

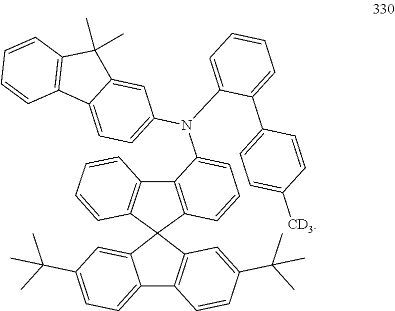 [SMILES: [2H]C([2H])([2H])c1ccc(-c2ccccc2N(c2ccc3c(c2)C(C)(C)c2ccccc2-3)c2cccc3c2-c2ccccc2C32c3cc(C(C)(C)C)ccc3-c3ccc(C(C)(C)C)cc32)cc1]

29. An organic electroluminescent device, comprising: an anode, a hole injection layer, a hole transport layer, a light-emitting layer, an electron transport layer, an electron injection layer and a cathode, wherein any one of the hole injection layer, the hole transport layer, the light-emitting layer, the electron transport layer, and the electron injection layer contains at least one compound, each of which is the novel organic electroluminescent compound according to claim 21.

30. The organic electroluminescent device according to claim 28, wherein the hole transport layer contains at least one compound, each of which is the novel organic electroluminescent compound according to claim 21.

31. An electronic display device, containing the organic electroluminescent device according to claim 30.
